(19) United States
(12) Patent Application Publication
(10) Pub. No.: US 2024/0409948 A1
(43) Pub. Date: Dec. 12, 2024

(54) CODON-OPTIMIZED HUMAN NPC1 GENES FOR THE TREATMENT OF NIEMANN-PICK TYPE C1 DEFICIENCY AND RELATED CONDITIONS

(71) Applicant: The United States of America,as represented by the Secretary,Department of Health and Human Services, Bethesda, MD (US)

(72) Inventors: Charles P. Venditti, Potomac, MD (US); William J. Pavan, Derwood, MD (US); Randy J. Chandler, Chevy Chase, MD (US)

(73) Assignee: The United States of America,as represented by the Secretary,Department of Health and Human Services, Bethesda, MD (US)

(21) Appl. No.: 18/816,498

(22) Filed: Aug. 27, 2024

Related U.S. Application Data
(63) Continuation of application No. 16/623,863, filed on Dec. 18, 2019, now Pat. No. 12,116,577.
(60) Provisional application No. 62/522,677, filed on Jun. 20, 2017.

(51) Int. Cl. C12N 15/67; C12N 15/52; C12N 15/62; C12N 15/86

(52) U.S. Cl. CPC C12N 15/67; C12N 15/52; C12N 15/62; C12N 15/86; C07K 2319/10; C12N 2710/10243; C12N 2799/022

(57) ABSTRACT

The present disclosure provides compositions for viral gene therapy, e.g. Adeno-Associated virus-directed gene therapy, and methods of using the same for the treatment and/or prevention of cholesterol storage diseases or disorders, such as Niemann-Pick disease, Type C.

### CROSS-REFERENCE TO RELATED APPLICATIONS

This application is a continuation of U.S. patent application Ser. No. 16/623,863, filed Dec. 18, 2019, which is the U.S. national stage of International Patent Application No. PCT/US2018/038584, filed Jun. 20, 2018, which claims the benefit of U.S. Provisional Patent Application No. 62/522,677, filed Jun. 20, 2017, each of which is incorporated herein by reference in its entirety for all purposes.

### STATEMENT REGARDING FEDERALLY SPONSORED RESEARCH OR DEVELOPMENT

This invention was made with Government support under project numbers HG-200318-11 and HG-000064-14, by the National Institutes of Health, National Human Genome Research Institute. The Government has certain rights in the invention.

### INCORPORATION BY REFERENCE OF MATERIAL SUBMITTED ELECTRONICALLY

Incorporated by reference in its entirety herein is a computer-readable nucleotide/amino acid sequence listing submitted concurrently herewith and identified as follows: One 207,325 Byte Extensible Markup Language (XML) file named “771507_ST26.xml,” dated Aug. 26, 2024.

### BACKGROUND OF THE INVENTION

Niemann-Pick disease, type C (NPC) is a rare and fatal, autosomal recessive, neurodegenerative disease that can present in infants, children, or adults. Its incidence in persons of Western European descent is 1/90,000 (Wassif CA et al., “High incidence of unrecognized visceral/neurological late-onset Niemann-Pick disease, type C1, predicted by analysis of massively parallel sequencing data sets,” Genet Med. 2015 Mar. 12). Approximately 95% of patients with NPC have mutations in NPC1, a gene implicated in intracellular cholesterol trafficking. Mutation of NPC1 causes intracellular accumulation of unesterified cholesterol in late endosomal/lysosomal structures and marked accumulation of glycosphingolipids, especially in neuronal tissue. Thus, NPC patients generally present with hepatosplenomegaly (enlargement of liver and spleen) and neurological degeneration.

A prenatal syndrome of nonimmune fetal hydrops can be the first symptom of NPC disease. Neonates can present with severe liver disease from infiltration of the liver and/or respiratory failure. Other infants, without liver or pulmonary disease, have hypotonia and developmental delay. The classic presentation occurs in mid-to-late childhood with the insidious onset of ataxia, vertical supranuclear gaze palsy (VSGP), and dementia. Regression is common. Seizures are frequent and neurological symptoms become disabling, making oral feeding impossible; death usually occurs in the late second or third decade from aspiration pneumonia. Adults can be more mildly affected and are more likely to present with dementia or psychiatric symptoms. There are no proven treatments for NPC, and after the diagnosis, fatal neurodegeneration is inevitable. The fact that most patients have disease onset in childhood makes the search for effective therapies urgent.

The diagnosis of NPC disease is confirmed by specialized biochemical testing that demonstrates cholesterol storage and is detected by filipin staining in cultured fibroblasts. Most individuals with NPC disease have NPC type 1, caused by mutations in NPC1; fewer than 20 individuals have been diagnosed with NPC type 2, caused by mutations in NPC2. Molecular genetic testing of NPC1 and NPC2 detects disease-causing mutations in approximately 94% of individuals with NPC disease, almost all of whom have mutations in NPC1. NPC disease, regardless of the locus and allele(s), is a recessive metabolic condition and the mutations are loss of function or reduced function. Therefore, providing and expressing a single copy of the wild type gene can completely restore NPC1 or 2 enzymatic function.

A series of landmark studies conducted by the research group of Dr. William Pavan of the NHGRI/NIH led to the identification of both the mouse and human genes for NPC1 (Loftus et al. Science 277:232-35; Carstea et al. Science 277:228-31). A murine model of NPC, Npc^{nih} (also called BALB/cNctr-Npc1^{m1N}/J), arising from a spontaneous frame-shift mutation in the Npc1 gene has been described and extensively characterized during these research efforts (Loftus et al. Science 277:232-35). Npenih homozygotes have an early, severe, and rapidly progressing disease, which is characterized by weight loss, ataxia, and lethality by 9 weeks of age. The mutation carried by this mouse is a null, and Npc^{nih} homozygous mice fail to make Npc1 protein or mRNA. This animal model also displays neurological symptoms and early lethality: Npc^{nih} homozygous mice uniformly begin losing weight by 6 weeks of age and do not survive past 9 weeks. Thus, these animals represent an ideal model of human NPC disease caused by loss of function mutations in the gene NPC1.

Over the years, other mouse models of NPC disease, specifically caused by varied natural or engineered mutations in the mouse Npc1 gene, have been generated but display less severe of a disease phenotype. All mouse models of NPC disease caused by mutation or other malfunction of the Npc1 gene in any mouse strain are treatable by the vector and derivatives described herein and are encompassed in the claims. Such models, as a group including Npc^{nih} homozygous animals, are generally considered Npc^{-/-} designating homozygous Npc loss-of-function alleles, of which Npc^{nih} is paradigmatic.

Notwithstanding the development of such mouse models, no curative therapy for NPC yet exists. A strategy or methodology for clinically treating NPC and/or providing a curative therapy for NPC and/or its symptoms is urgently needed in the art. The present disclosure fulfills such a need.

### BRIEF SUMMARY OF THE INVENTION

The present disclosure provides gene therapy vectors (e.g., Adeno-Associated Virus (AAV) gene therapy vectors) comprising a therapeutic human nucleic acid molecule which is able to ameliorate, treat, and/or correct the cellular defect characteristic of certain cholesterol storage diseases or disorders, such as, Niemann-Pick disease type C (“NPC”). The gene therapy vectors of the present disclosure comprise NPC1 alleles that are codon optimized in order to maximize human expression (_{co}NPC1).

The alleles may be a fusion protein that includes a tag (e.g., a FLAG® tag, such as a 3xFLAG® tag (Sigma, St. Louis, MO), which can afford facile detection, for example to determine biodistribution of the _{co}NPC1. The alleles may be part of a fusion protein that includes a protein transduction domain peptide (PTD), which allows trans-cellular correction (i.e., the transfer of the allele and/or expressed therapeutic protein to neighboring cells that have not received or been infected with the therapy vector; a/k/a “cross correction”). In certain embodiments, the alleles are part of a fusion protein that comprises a PTD (Tat, TP2, and/or TP10) and a tag (e.g., FLAG® tag).

The gene therapy vectors in accordance with the present disclosure may include a truncated Elongation Factor 1 alpha (EF1α) promoter (EF1αₜ) (SEQ ID NO: 10) at the 5′ end of NPC1. The EF1αₜ promoter is a 227 base pair truncated EF1α promoter that retains activity. Additional promoters that are suitable for the inventive gene therapy vectors include the 231 base pair mini EF1α promoter (mini EF1α) (SEQ ID NO: 51) and the 231 base pair short EF1α promoter, also called EF1α S (SEQ ID NO: 52).

The gene therapy vector may include at least one of: a strong translation initiation site; multiple stop signals/codons at the end of the NCP1 allele or the fusion protein (e.g., coNCP1-PTD, coNCP1-3xFLAG® tag, coNCP1-PTD-3xFLAG® tag, wherein the PTD can be any protein transduction domain, such as Tat, TP2, or TP10); or both. The use of multiple stop signals/codons ensures maximal NPC1 gene or fusion protein expression and protein production in the cell and prevents aberrant read-through.

The vector constructs of the present disclosure may be utilized for gene therapy of certain cholesterol storage diseases or disorders, for example Niemann-Pick disease, type C (NPC).

Examples provided herein demonstrate the reduction of practice and the effectiveness of the compositions and methods of the present disclosure in the most established and well-characterized animal model of NPC.

In particular, according to a further aspect, the present disclosure provides compositions and methods for ameliorating, treating and/or preventing at least one symptom of cholesterol storage diseases or disorders (e.g., diseases associated with accumulated cellular cholesterol). In certain aspects, the present disclosure provides compositions and methods for ameliorating, treating, and/or preventing at least one symptom of Niemann-Pick disease, type C. In certain embodiments, the present disclosure relates to compositions and methods for ameliorating, treating, and/or preventing at least one symptom of cholesterol storage diseases or disorders that are characterized by or associated with a risk of diminution of central nervous system (CNS) function, for example NPC. In still other embodiments, the present disclosure relates to nucleic acid molecules encoding therapeutic transgenes, e.g., NPC1, which has been codon optimized for expression in humans and/or human cell lines, and is capable of restoring the function loss to one or more defective genes or polypeptide products thereof, e.g., a mutant NPC1 gene. In yet other embodiments, the disclosure relates to pharmaceutical compositions that are suitable for administering therapeutically effective amounts the nucleic acid molecules of the present disclosure. In still further embodiments, the present disclosure relates to methods for diagnosing NPC and/or monitoring the progress of gene therapy treatment of NPC by monitoring the expression and/or function of a therapeutic gene, e.g., NPC1.

According to yet a further aspect, the present disclosure relates to an engineered cell line that expresses (e.g., overexpresses) NPC1 protein, the cell line comprising the construct or gene therapy vector of the present disclosure comprising the _{co}NPC1 or the fusion protein of the present disclosure. In some embodiments, the protein produced from the engineered cell line may be utilized in at least one of: enzyme replacement therapy, to produce exosomes containing the NPC1 protein, or a combination thereof.

The present disclosure, in further embodiments, relates to methods of gene therapy involving administering in an effective amount a nucleic acid molecule comprising a therapeutic transgene, e.g., _{co}NPC1, in order to ameliorate, treat, and/or prevent at least one symptom of a cholesterol storage disease or disorder, such as NPC related to an NPC1 mutation. In still other embodiments, the present disclosure relates to methods of gene therapy involving administering, in an effective amount, an expression vector encoding _{co}NPC1 in order to ameliorate, treat, and/or prevent at least one symptom of a cholesterol storage disease or disorder, for example NPC disease. In yet other embodiments, the nucleic acid molecule and/or expression vector of the present disclosure may be selectively delivered to a target site or tissue, e.g., the central nervous system.

The nucleic acid molecules or gene therapy constructs in certain embodiments comprise one or more therapeutic transgenes, e.g., NPC1, which is codon optimized for expression in humans and is under the control of at least one genetic regulatory element, such as a promoter. In certain embodiments, the promoter is a tissue-specific promoter that is capable of being expressed in the CNS. In some embodiments, the promoter is a truncated EF1α promoter (EF1αₜ) (SEQ ID NO: 10). In other embodiments, the promoter is the mini EF1α promoter (SEQ ID NO: 51) or the short EF1α promoter, EF1α S (SEQ ID NO: 52).

The present disclosure also relates to specific nucleic acid molecules comprising a therapeutic transgene, e.g., _{co}NPC1, under transcriptional control of a promoter that is capable of being expressed in the CNS. The disclosure also contemplates that such nucleic acid constructs may be engineered into any suitable gene therapy vector, such as a retrovirus, lentivirus adenovirus, or AAV vector (e.g., the AAV vector can be of any serotype, including, but not limited to serotype 9, PHP.B or derivatives thereof, and Anc80 or derivatives thereof), nucleic acid such as plasmid DNA, peptide nucleic acids, or mRNA, including mRNAs that contain modified bases to enhance in vivo expression. All forms of nucleic acids can be delivered without further modification, such as naked DNA, or packaged into nanoparticles or lipid nanoparticles and delivered in an appropriate fashion to produce NPC1 expression. In a particular embodiment, the background gene therapy vector is an AAV. In a particular embodiment, a therapeutic exosome is administered to a subject, wherein the therapeutic exosome comprises at least one of the therapeutic transgene as described herein (e.g., coNPC1 gene, which may be translationally fused with at least one of a PTD, a tag moiety, or a combination thereof), NPC1 protein, or a combination thereof.

The foregoing and other features and advantages of the disclosure will become more apparent from the following detailed description and accompanying drawings. Those skilled in the art will appreciate that the utility of this disclosure is not limited to the specific experimental modes and materials described herein.

In a preferred embodiment, the present disclosure is directed to a nucleic acid construct for the expression of a therapeutic amount of NPC1 in a cell, the construct comprising a human codon-optimized NPC1 gene selected from the group consisting of SEQ ID NOs: 1-8.

In certain embodiments, the NPC1 is translationally fused to a protein transduction domain (PTD) to form a NPC1-PTD fusion protein, wherein the fusion protein is capable of cross-correcting non-transformed neighboring cells.

The protein transduction domain can be HIV-Tat, transportin 10 (TP10), or TP2.

The _{co}NPC1 gene can be under the control of a promoter.

The promoter can be an EF1α truncated promoter (EF1αₜ).

The promoter can be a mini EF1α promoter (EF1α promoter).

The promoter can be a short EF1α promoter (EF1α S).

The NPC1 of the nucleic acid construct can be translationally fused to a FLAG®-tag moiety (e.g., 1x-FLAG® tag or 3x-FLAG® tag).

The nucleic acid construct can be expressed in a transduced cell and the neighboring cells, each of which can be neuronal cells.

The nucleic acid construct can comprises a nucleic acid sequence selected from the group consisting of SEQ ID NOS: 1-8.

An expression vector comprising a nucleic acid construct of the present disclosure can be capable of expressing the NPC1 protein, the NPC1-PTD fusion protein, the NPC1-FLAG® tag fusion protein, the NPC1-PTD-FLAG® tag fusion protein, or the NPC1-FLAG® tag-PTD fusion protein in a cell.

A cell can comprise the expression vector, e.g., the cell can produce the expression

vector and/or the cell can produce NPC1, such as a therapeutic exosome having NPC1 protein.

The expression vector can be used to treat Niemann-Pick disease, type C1 in a subject. The method can comprise administering a therapeutically effective amount of an expression vector comprising a nucleic acid molecule encoding an NPC1-PTD fusion protein, wherein the NPC1 gene is a human codon-optimized NPC1 gene selected from the group consisting of SEQ ID NOs: 1-8 and wherein the fusion protein is capable of cross-correcting non-transformed neighboring cells.

The PTD protein transduction domain can be HIV Tat, TP10, or TP2.

The NPC1 or fusion gene can be under the control of a promoter.

The promoter can be an EF1ₜ promoter.

The NPC1 can be translationally fused to a FLAG®-tag moiety (e.g., a 1x-FLAG® tag or 3xFLAG® tag) to form a NPC1-FLAG® tag fusion protein, a NPC1-PTD-FLAG® tag fusion protein, or a NPC1-FLAG® tag-PTD fusion protein.

The cell transduced with the NPC1 gene or the fusion gene, and the neighboring cells can be neuronal cells.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure may be utilized in numerous combinations, all of which are expressly contemplated by the present description. These additional advantages, objects and embodiments are expressly included within the scope of the present disclosure. The publications and other materials used herein to illuminate the background of the disclosure, and in particular cases, to provide additional details respecting the practice, are incorporated by reference in their entirety for all purposes.

### BRIEF DESCRIPTION OF THE DRAWINGS

The following detailed description, given by way of example, but not intended to limit the disclosure solely to the specific embodiments described, may best be understood in conjunction with the accompanying drawings. Reference will now be made, by way of example, to the accompanying drawings which show example embodiments of the present disclosure.

FIG. 1A depicts a nucleotide sequence of codon optimized NPC1 (SEQ ID NO: 1).

FIG. 1B depicts a nucleotide sequence of codon optimized NPC1-Tat (SEQ ID NO: 2).

FIG. 1C depicts a nucleotide sequence of codon optimized NPC1-3xFLAG® tag (SEQ ID NO: 3).

FIG. 1D depicts a nucleotide sequence of codon optimized NPC1-TP10 (SEQ ID NO: 4).

FIG. 1E depicts a nucleotide sequence of codon optimized NPC1-TP2 (SEQ ID NO: 5).

FIG. 1F depicts a nucleotide sequence of codon optimized NPC1-3xFLAG® tag-TAT (SEQ ID NO: 6).

FIG. 1G depicts a nucleotide sequence of codon optimized NPC1-3xFLAG® tag-TP10 (SEQ ID NO: 7).

FIG. 1H depicts a nucleotide sequence of codon optimized NPC1-3xFLAG® tag-TP2) SEQ ID NO: 8.

FIGS. 2A-2D depict the results of a BLASTN sequence comparison between wildtype NPC1 (SEQ ID NO: 9) and the codon optimized NPC1 gene (SEQ ID NO: 1), in consecutive order from the 5′ end to the 3′ end. The sequence comparison starts at FIG. 2A, which shows the 5′ end of the sequences, and continues in order from 5′ in the direction of the 3′ end of the sequences from FIG. 2A, to FIG. 2B, to FIG. 2C, and the 3′ ends of the sequences are shown at the end of FIG. 2D.

FIG. 3 is a schematic showing the strategy for anti-FLAG tag/Red transfection and filipin staining after transfection with the the pAAV-mini EF1α NPC1 plasmid in U2OS-NPC1-null cells. Cells were plated in 8 wells, numbered 1-8, for low confluence and staining was determined four days post-transfection. [A] is no primary; Anti-rabbit-red secondary 1:200. [B] is Anti-FLAG tag primary, 1:200; Anti-rabbit-red secondary 1:200. [C] is No primary; Anti-rabbit-red secondary 1:200. [D] is Anti-FLAG tag primary, 1:200; Anti-rabbit-red secondary, 1:200.

FIGS. 4A-4C are enlarged microscopic images of the same field of well 2 in FIG. 3; U2OS-NPC1-null cells, four days post-transfection with pAAV-mini EF1α coNPC1-3xFLAG tag plasmid, stained with filipin and anti-FLAG tag antibody. The areas showing accumulation of white particles in FIG. 4A are areas of filipin accumulation. The areas in FIG. 4B showing light gray and white accumulation are cells that highly expressed the _{co}NPC1-3xFLAG tag after transfection with pAAV-mini EF1α-coNPC1-3xFLAG tag plasmid, and the FLAG tag epitope has been detected by the anti-FLAG tag antibody. FIG. 4C shows both the filipin staining and anti-FLAG tag antibody. Arrows indicate cells showing less accumulated filipin (less white) in cells that have been transfected. This demonstrates that the _{co}NPC1 expression via the _{co}NPC1-3xFLAG tag plasmid is effective at reducing/removing accumulated cellular cholesterol.

FIGS. 4D-4F are enlarged microscopic images of the same field of well 2 in FIG. 3; U2OS-NPC1-null cells, four days post-transfection with pAAV-mini EF1α-coNPC1-3xFLAG tag plasmid, stained with filipin and anti-FLAG tag antibody. The areas showing accumulation of white particles in FIG. 4D are areas of filipin accumulation. The areas in FIG. 4E showing light gray and white accumulation are cells that highly expressed the _{co}NPC1-3xFLAG tag after transfection with pAAV-coNPC1-3xFLAG tag plasmid, and the FLAG tag epitope has been detected by the anti-FLAG tag antibody. FIG. 4F shows both the filipin staining and anti-FLAG tag antibody. Arrows indicate cells showing less accumulated filipin (less white) in cells that have been transfected. This demonstrates that the _{co}NPC1 expression via the _{co}NPC1-3xFLAG tag plasmid is effective at reducing/removing accumulated cellular cholesterol.

FIGS. 4G-4I are enlarged microscopic images of the same field of well 2 in FIG. 3; U2OS-NPC1-null cells, four days post-transfection with pAAV-mini EF1α coNPC1-3xFLAG tag plasmid, stained with filipin and anti-FLAG tag antibody. The areas showing accumulation of white particles in FIG. 4G are areas of filipin accumulation. The areas in FIG. 4H showing light gray and white accumulation are cells that highly expressed the _{co}NPC1-3xFLAG tag after transfection with pAAV-mini EF1α-coNPC1-3xFLAG tag plasmid, and the FLAG tag epitope has been detected by the anti-FLAG tag antibody. FIG. 4I shows both the filipin staining and anti-FLAG tag antibody. Arrows indicate cells showing less accumulated filipin (less white) in cells that have been transfected. This demonstrates that the _{co}NPC1 expression via the _{co}NPC1-3xFLAG tag plasmid is effective at reducing/removing accumulated cellular cholesterol.

FIGS. 4J-4L are enlarged microscopic images of the same field of well 2 in FIG. 3; U2OS-NPC1-null cells, four days post-transfection with pAAV-mini EF1α coNPC1-3xFLAG tag plasmid, stained with filipin and anti-FLAG tag antibody. The areas showing accumulation of white particles in FIG. 4J are areas of filipin accumulation. The areas in FIG. 4K showing light gray and white accumulation are cells that highly expressed the _{co}NPC1-3xFLAG tag after transfection with pAAV-mini EF1α-coNPC1-3xFLAG tag plasmid, and the FLAG tag epitope has been detected by the anti-FLAG tag antibody. FIG. 4L shows both the filipin staining and anti-FLAG tag antibody. Arrows indicate cells showing less accumulated filipin (less white) in cells that have been transfected. This demonstrates that the _{co}NPC1 expression via the _{co}NPC1-3xFLAG tag plasmid is effective at reducing/removing accumulated cellular cholesterol.

FIGS. 4M-4O are enlarged microscopic images of the same field of well 2 in FIG. 3; U2OS-NPC1-null cells, four days post-transfection with pAAV-mini EF1α-coNPC1-3xFLAG tag plasmid, stained with filipin and anti-FLAG tag antibody. The areas showing accumulation of white particles in FIG. 4M are areas of filipin accumulation. The areas in FIG. 4N showing light gray and white accumulation are cells that highly expressed the _{co}NPC1-3xFLAG tag after transfection with pAAV-coNPC1-3xFLAG tag plasmid, and the FLAG tag epitope has been detected by the anti-FLAG tag antibody. FIG. 4O shows both the filipin staining and anti-FLAG tag antibody. Arrows indicate cells showing less accumulated filipin (less white) in cells that have been transfected. This demonstrates that the _{co}NPC1 expression via the _{co}NPC1-3xFLAG tag plasmid is effective at reducing/removing accumulated cellular cholesterol.

FIGS. 4P-4R are enlarged microscopic images of the same field of well 2 in FIG. 3; U2OS-NPC1-null cells, four days post-transfection with pAAV-mini EF1α coNPC1-3xFLAG tag plasmid, stained with filipin and anti-FLAG tag antibody. The areas showing accumulation of white particles in FIG. 4P are areas of filipin accumulation. The areas in FIG. 4Q showing light gray and white accumulation are cells that highly expressed the _{co}NPC1-3xFLAG tag after transfection with pAAV-mini EF1α-coNPC1-3xFLAG tag plasmid, and the FLAG tag epitope has been detected by the anti-FLAG tag antibody. FIG. 4R shows both the filipin staining and anti-FLAG tag antibody. Arrows indicate cells showing less accumulated filipin (less white) in cells that have been transfected. This demonstrates that the _{co}NPC1 expression via the _{co}NPC1-3xFLAG tag plasmid is effective at reducing/removing accumulated cellular cholesterol.

FIGS. 5A-5C are enlarged microscopic images of the same field of well 3 in FIG. 3: U2OS-NPC1-null cells, four days post-transfection with hMITF-FLAG tag plasmid (as negative control). The areas in FIG. 5A showing accumulation of white particles are areas of filipin accumulation. The areas in FIG. 5B showing light gray and white accumulation are cells that highly expressed the MITF-FLAG tag after transfection with hMITF-FLAG tag plasmid, and the FLAG tag epitope has been detected by the anti-FLAG antibody. FIG. 5C shows both showing both the filipin staining and anti-FLAG tag antibody. This shows that filipin is present in cells that have been transfected with hMITF-FLAG tag plasmid.

FIGS. 5D-5F are enlarged microscopic images of the same field of well 3 in FIG. 3: U2OS-NPC1-null cells, four days post-transfection with hMITF-FLAG tag plasmid (as negative control). The areas in FIG. 5D showing accumulation of white particles are areas of filipin accumulation. The areas in FIG. 5E showing light gray and white accumulation are cells that highly expressed the MITF-FLAG tag after transfection with hMITF-FLAG tag plasmid, and the FLAG tag epitope has been detected by the anti-FLAG antibody. FIG. 5F shows both the filipin staining and anti-FLAG tag antibody. This shows that filipin is present in cells that have been transfected with hMITF-FLAG tag plasmid.

FIGS. 5G-5I are enlarged microscopic images of the same field of well 3 in FIG. 3: U2OS-NPC1-null cells, four days post-transfection with hMITF-FLAG tag plasmid (as negative control). The areas in FIG. 5G showing accumulation of white particles are areas of filipin accumulation. The areas in FIG. 5H showing light gray and white accumulation are cells that highly expressed the MITF-FLAG tag after transfection with hMITF-FLAG tag plasmid, and the FLAG tag epitope has been detected by the anti-FLAG antibody. FIG. 5I shows both showing both the filipin staining and anti-FLAG tag antibody. This shows that filipin is present in cells that have been transfected with hMITF-FLAG tag plasmid.

FIGS. 5J-5L are enlarged microscopic images of the same field of well 3 in FIG. 3: U2OS-NPC1-null cells, four days post-transfection with hMITF-FLAG tag plasmid (as negative control). The areas in FIG. 5J showing accumulation of white particles are areas of filipin accumulation. The areas in 5K showing light gray and white accumulation are cells that highly expressed the MITF-FLAG tag after transfection with hMITF-FLAG tag plasmid, and the FLAG tag epitope has been detected by the anti-FLAG antibody. FIG. 5L shows both the filipin staining and anti-FLAG tag antibody. This shows that filipin is present in cells that have been transfected with hMITF-FLAG tag plasmid.

FIGS. 5M-5O are enlarged microscopic images of the same field of well 3 in FIG. 3: U2OS-NPC1-null cells, four days post-transfection with hMITF-FLAG tag plasmid (as negative control). The areas in FIG. 5M showing accumulation of white particles are areas of filipin accumulation. The areas in FIG. 5N showing light gray and white accumulation are cells that highly expressed the MITF-FLAG tag after transfection with hMITF-FLAG tag plasmid, and the FLAG tag epitope has been detected by the anti-FLAG antibody. FIG. 5O shows both showing both the filipin staining and anti-FLAG tag antibody. This shows that filipin is present in cells that have been transfected with hMITF-FLAG tag plasmid.

FIGS. 5P-5R are enlarged microscopic images of the same field of well 3 in FIG. 3: U2OS-NPC1-null cells, four days post-transfection with hMITF-FLAG tag plasmid (as negative control). The areas in FIG. 5P showing accumulation of white particles are areas of filipin accumulation. The areas in FIG. 5Q showing light gray and white accumulation are cells that highly expressed the MITF-FLAG tag after transfection with hMITF-FLAG tag plasmid, and the FLAG tag epitope has been detected by the anti-FLAG antibody. FIG. 5R shows both showing both the filipin staining and anti-FLAG tag antibody. This shows that filipin is present in cells that have been transfected with hMITF-FLAG tag plasmid.

FIG. 6 is an image of a gel that demonstrates that human NPC1 knock-out cells (U20S c cells) transfected with AAV mini EF1α coNPC1 3xFLAG tag (FIG. 8B), AAV mini EF1α-coNPC1-tat (FIG. 8C), AAV mini EF1α-coNPC1-TP10 (FIG. 8C), AAV mini EF1α-coNPC1-TP2 (FIG. 8C), AAV mini EF1α-coNPC1 3xFLAG tag-tat (FIG. 8D), and AAV mini EF1α-coNPC1 3xFLAG tag-TP2 (FIG. 8D) constructs produced robust expression of NPC1, which was comparable to Npc1 expression in wild-type mouse liver.

FIG. 7A is an enlarged microscopic image of an area of U2OS-NPC1-null cells, four days post-transfection with the pAAV-EF1α S-intronS-coNPC1 plasmid, stained with filipin. The areas showing accumulation of white particles are areas of filipin accumulation. Arrow indicates cell without accumulation.

FIG. 7B is an enlarged microspic image of the area shown in FIG. 7A of U2OS-NPC1-null cells, four days post-transfection with the pAAV-EF1α S-intronS-coNPC1 plasmid. The areas showing light gray and white accumulation indicates coNPC1 protein which was detected by the anti-NPC1 antibody. Arrow indicates cell with NPC1 staining.

FIG. 7C is an enlarged microscopic image of the area shown in FIGS. 7A and 7B showing the filipin staining, anti-NPC1 antibody, and TOPRO-31 (a nuclear marker). FIG. 7C shows less accumulated filipin in cells that express NPC1 protein indicated by arrow. This demonstrates that the _{co}NPC1 expression via the pAAV-EF1-intronS-coNPC1 plasmid is effective at reducing/removing accumulated cellular cholesterol.

FIG. 7D is an enlarged microscopic image of an area of U2OS-NPC1-null cells, four days post-transfection with pAAV-EF1α S-intronS-coNPC-3xFLAG tag plasmid, stained with filipin. The areas showing accumulation of white particles are areas of filipin accumulation. Arrow indicates cell without accumulation.

FIG. 7E is an enlarged microscopic image of an area of U2OS-NPC1-null cells, four days post-transfection with the pAAV-EF1α S-intronS-coNPC-3xFLAG tag plasmid. The areas showing light gray and white accumulation are coNPC-3xFLAG tag protein which was detected by the anti-NPC1 antibody. Arrow indicates cell with NPC1 staining.

FIG. 7F is an enlarged microscopic image of the area shown in FIGS. 7D and 7E showing both the filipin staining, anti-NPC1 antibody, and TOPRO-31 (a nuclear marker). FIG. 7F shows less accumulated filipin less accumulated filipin in cells that express NPC1 protein indicated by arrow. This demonstrates that the _{co}NPC1 expression via the pAAV-EF1-intronS-coNPC1 plasmid is effective at reducing/removing accumulated cellular cholesterol.

FIG. 7G is an enlarged microscopic image of an area of U2OS-NPC1-null cells, four days post-transfection with the pAAV-EF1α S-intronS-coNPC1-TAT plasmid, stained with filipin. The areas showing accumulation of white particles are areas of filipin accumulation. Arrow indicates cell without accumulation.

FIG. 7H is an enlarged microscopic image of the area shown in FIG. 7G of U2OS-NPC1-null cells, four days post-transfection with the pAAV-EF1αS-intronS-coNPC1-TAT plasmid. The areas showing white accumulation are NPC1-TAT proteins which were detected by the anti-NPC1 antibody. Arrow indicates cell with NPC1 staining.

FIG. 7I is an enlarged microscopic image of the same field shown in FIGS. 7G and 7H showing the filipin staining, the anti-NPC1 antibody, and TOPRO-31 (a nuclear maker). FIG. 7I shows less accumulated filipin less accumulated filipin in cells that express NPC1 protein indicated by arrow. This demonstrates that the _{co}NPC1 expression via the pAAV-EF1αS-intronS-coNPC1-TAT plasmid is effective at reducing/removing accumulated cellular cholesterol. Cross-correction is shown as bright specks in the top-right quadrant of FIG. 7I.

FIG. 8A is a schematic of the experimental design of a “second generation” vector which includes a mini EF1α promoter, a codon-optimized NPC1 gene, and a rabbit beta globin gene polyA signal.

FIG. 8B is a schematic of the experimental design of a “second generation” vector which includes a mini EF1α promoter, a codon-optimized NPC1 gene, a 3x FLAG tag moiety, and a rabbit beta globin gene polyA signal.

FIG. 8C is a schematic of the experimental design of a “second generation” vector which includes a mini EF1α promoter, a codon-optimized NPC1 gene, a protein transduction domain (either TAT, TP2, or TP10), anda rabbit beta globin gene polyA signal.

FIG. 8D is a schematic of the experimental design of a “second generation” vector which includes a mini EF1α promoter, a codon-optimized NPC1 gene, a 3x FLAG tag moiety, a protein transduction domain, and a rabbit beta globin gene polyA signal.

FIG. 9 is a schematic showing the differences between the first and second generation (top) AAV gene therapy vectors and the third and fourth generation (bottom) AAV gene therapy vectors. In the first and second generation vectors, the duplicated 59 base pair sequence from the wild-type (“WT”) AAV2 vector is present. and the Inverted Terminal Repeats (shown as the ′5 ITR and the ′3 ITR) are 130 base pairs long (SEQ ID NO: 48). In the third and fourth generation vectors, the duplicated 59 base pair sequence (SEQ ID NO: 50) from the wild-type (“WT”) AAV2 vector is deleted (shown as dotted lines). The third and fourth generation vectors further include a 30 base pair miscellaneous sequence and a 21 base pair miscellaneous sequence flanking the transgene, and the ITRs (shown as the ′5 ITR and the ′3 ITR) are 145 base pairs long (SEQ ID NO: 49).

FIG. 10A is a schematic of the experimental design of a “third generation” vector which includes the short EF1α promoter, a codon-optimized NPC1 gene, and a rabbit beta globin gene polyA signal.

FIG. 10B is a schematic of the experimental design of a “third generation” vector which includes the short EF1α promoter, a codon-optimized NPC1 gene, a 3x FLAG tag moiety, and a rabbit beta globin gene polyA signal.

FIG. 10C is a schematic of the experimental design of a “third generation” vector which includes the short EF1α promoter, a codon-optimized NPC1 gene, a TAT protein transduction domain, and a rabbit beta globin gene polyA signal.

FIG. 10D is a schematic of the experimental design of a “third generation” vector which includes the short EF1α promoter, a codon-optimized NPC1 gene, a 3x FLAG tag moiety, a TAT protein transduction domain, anda rabbit beta globin gene polyA signal.

FIG. 11A is a schematic of the experimental design of a “fourth generation” vector which includes the short EF1α promoter, a synthetic intron (“Intron S”), a codon-optimized NPC1 gene, and a rabbit beta globin gene polyA signal.

FIG. 11B is a schematic of the experimental design of a “fourth generation” vector which includes the short EF1α promoter, a synthetic intron (“Intron S”), codon-optimized NPC1 gene, a 3x FLAG tag moiety, and a rabbit beta globin gene polyA signal.

FIG. 11C is a schematic of the experimental design of a “fourth generation” vector which includes the short EF1α promoter, a synthetic intron (“Intron S”), a codon-optimized NPC1 gene, a TAT protein transduction domain, and a rabbit beta globin gene polyA signal.

### DETAILED DESCRIPTION OF THE INVENTION

The present disclosure is directed, at least in part, to compositions and methods for ameliorating, treating, and/or preventing at least one symptom of a cholesterol storage disease or disorder, such as Niemann-Pick disease, type C (“NPC”) caused by mutation or malfunction of the NPC1 enzyme, which is encoded by the NPC1 gene.

The present disclosure relates to a particular set of newly developed expression constructs for the therapeutic expression of NPC1 for treating Niemann-Pick disease, type C (NPC) caused as a result of mutations in the NPC1 gene.

Niemann-Pick disease, type C (NPC) is a rare and fatal, autosomal recessive, neurodegenerative disease that can present in infants, children, or adults, and which causes progressive neurological degeneration that ultimately leads to disability and premature death.

Approximately 95% of NPC patients have mutations in the NPC1 gene, which is a gene encoding a 1278 amino acid transmembrane protein that is involved in the intracellular trafficking of cholesterol. NPC1 patients typically exhibit an impairment in the esterification of low-density lipoprotein (LDL) cholesterol and reduced trafficking of unesterified cholesterol to the plasma membrane, thereby leading to an accumulation of cholesterol in the cells, and in particular, in neuronal tissues. Intracellular cholesterol levels may be detected/examined by filipin staining. That is, filipin staining is directly related to the level of cholesterol in a cell.

The inventors previously constructed a series of therapeutic adeno-associated virus (AAV) vectors for expressing the human NPC1 gene in cells, such as neurons, as a gene therapy based treatment for NPC1 disease, such as NPC related to an NPC1 mutation. Specifically, the inventors constructed and tested two AAV9-based vectors expressing the human NPC1 gene under control of either the EF1α or CamKII promoters and demonstrated in Npc1^{-/-} mouse models that AAV-based gene therapy corrected mutant NPC1-based cholesterol accumulation and thus “represents a viable and efficacious therapeutic approach for NPC1 disease.” See Chandler et al., “Systemic AAV9 gene therapy improves the lifespan of mice with Niemann-Pick disease, type C1,” Hum Mol Genet. 1;26(1): 52-64 (January 2017), which is incorporated by reference herein.

The present disclosure describes a new set of improved AAV gene therapy vectors that contain a codon optimized NPC1 allele, referred to herein as second, third, and fourth generation vectors. The second and third generation vectors include: (1) codon optimized NPC1 allele to maximize human expression and enable detection of a unique nucleic acid sequence in a human or other specie; (2) a codon optimized NPC1 fused to a 3xFLAG®-tag to facilitate easy analysis of intracellular biodistribution of NPC1; (3) a codon optimized NPC1 fused to a protein transduction domain (e.g., TAT, TP10, and TP2) and a 3xFLAG®-tag to allow for cross-correction; and (4) a codon optimized NPC1 fused only to a protein transduction domain (e.g., TAT, TP10, and TP2) to allow for cross-correction. The fourth generation vectors further include a synthetic intron (“intron S,” SEQ ID NO: 25).

The new set of improved AAV gene therapy vectors of the present disclosure may further contain a nucleotide sequence encoding an antibiotic resistance marker. The antibiotic resistance marker may be an ampicillin resistance marker or a kanamycin resistance marker.

In addition, the new vector constructs can utilize a truncated EF1α promoter (EF1ₜ), a mini EF1α promoter, or a short EF1α promoter, a stronger translation initiation site at the 5′ end of NPC1 gene, and/or multiple stop signals to ensure maximal NPC1 gene expression and protein production in the cell.

In accordance with the present disclosure, each of the constructs was synthesized and tested for NPC1 expression in a human NPC1-null mutant cell. In addition, the ability of the expressed NPC1 variants to clear disease-associated cholesterol accumulation was tested using a filipin staining assay. At least one construct—mini EF1α-_{co}NPC1-3xFLAG® tag—was prepared as an AAV9 virus vector.

The constructs comprising NPC1 fused at their C-terminal ends to protein transduction domains (e.g., TAT, TP10, and TP2) have positive NPC1 expression in cells. According to this aspect, a cell abutting or touching the transduced cell would now also have the vector expressing NPC1 present because the protein transduction domain will pull the fusion protein from the cytosol across the cell membrane and then into contact with other local cells that were not directly transduced to transfer NCP1. In this way, the PTD fusion alleles of NPC1 may allow a cell that has not received the AAV viral genome to nevertheless have the expressed fusion enzyme present.

In accordance with the present disclosure, the “cross correction” or “trans-cellular correction” is aspect is an improvement on earlier NPC1 gene therapy approaches, which require individual cellular correction. The herein disclosed vector embodiments would allow cross correction of local or proximal cells not otherwise directly transformed by an expression vector and thereby increase the potency of the NPC1-based gene therapy approach. Cross-correction or trans-cellular correction can refer to the transfer of the allele and/or expressed therapeutic protein to neighboring cells that have not been directly transformed by an expression vector described here.

The present disclosure provides constructs that represent a new suite of therapeutic agents to treat human NPC1 deficiency and related conditions. The NPC1 alleles may be easily and uniquely detected in preclinical models and patients given that they are synthetic and easily tested. Furthermore, the vectors could be used to treat NPC1 deficiency by systemic or targeted gene delivery, including into the cerebral spinal fluid or brain structures—which are typically affected by NPC1 patients.

In certain aspects, compositions of the instant disclosure include one or more gene therapy vectors (e.g., AAV vectors of various serotypes, including but not limited to serotypes 9, PHP.B, Anc serotypes including Anc80, rh10 and related neuronotrophic capsids) comprising a therapeutic human nucleic acid molecule which is able to correct the cellular defect characteristic of certain cholesterol storage diseases or disorders, such as, Niemann-Pick disease type C (“NPC”). The gene therapy vectors comprise NPC1 alleles that are codon optimized in order to maximize human expression (coNPC1).

The alleles of the present disclosure may also be tagged in order to afford facile detection. For example, the tags can be used in biodistribution analysis. The alleles of the present disclosure may be fused with a tag (e.g., 1xFLAG® tag or 3xFLAG® tag). In other embodiments, the alleles of the present disclosure may be fused with a tag and a protein transduction domain, which allow for trans-cellular correction.

The gene therapy vectors in accordance with the present disclosure may comprise an improved and truncated promoter (EF1αₜ), and/or a stronger translation initiation site at the 5′ end of NPC1. The gene therapy vectors of the present disclosure may comprise multiple stop signals, thereby ensuring maximal NPC1 gene expression and protein production in the cell.

The gene therapy vectors of the present disclosure may further contain a synthetic intron (“intron S,” SEQ ID NO: 25), which may increase splicing and expression of the transgene.

The gene therapy vectors of the present disclosure may comprise full-length, wild type AAV inverted terminal repeats (ITRs) that flank the transgene. Besides the ITRs, the nucleic acid construct may be devoid of any other sequence from the wild-type AAV genome, which may lessen or eliminate genotoxicity of the vectors, as described in Logan et al., “Identification of liver-specific enhancer-promoter activity in the 3′ untranslated region of the wild-type AAV2 genome,” Nat Genet. 49(8):1267-1273 (August 2017), which is incorporated by reference herein.

In order to facilitate review of the various embodiments of the disclosure, the following definitions of terms and explanations of abbreviations are provided, as follows:

### Definitions

The instant disclosure provides for the therapeutic or prophylactic use of gene therapy vectors to achieve treatment of subjects having or at risk of developing a cholesterol storage disease or disorder. In certain embodiments, the disclosure provides compositions and methods for ameliorating, treating, and/or preventing Niemann-Pick disease, type C1, either by delivery of the vector to the CNS in a targeted manner, or systemically, using a recombinant AAV viral vector (such as, but not limited to, serotypes 9, PHP.B, Anc80, and related capsids) that contain an NPC1 allele as described herein. In related embodiments, the codon optimized NPC1 allele as described herein is used to maximize human expression. In another embodiment, the codon optimized NPC1 described herein is fused to a tag, such as a 3xFLAG®-tag, which can be used to examine intracellular biodistribution of NPC1. In a further embodiment, the codon optimized NPC1 described herein may be fused to a protein transduction domain (e.g., Tat, TP10, and TP2) and/or a 3xFLAG®-tag to allow for cross-correction. In another embodiment, the codon optimized NPC1 of the present disclosure is fused to a protein transduction domain (e.g., Tat, TP10, and TP2) to allow for cross-correction.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

### General Terms

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or”, as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “obtaining” is understood herein as manufacturing, purchasing, or otherwise coming into possession of.

As used herein, “kits” are understood to contain at least the non-standard laboratory reagents of the present disclosure and one or more non-standard laboratory reagents for use in the methods of the disclosure.

The terms “about” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 10- or 5-fold, and more preferably within 2-fold of a given value. Numerical quantities given herein are approximate unless stated otherwise, meaning that the term “about” or “approximately” can be inferred when not expressly stated.

### Terms Relating to NPC

The term Niemann-Pick disease, Type C or abbreviated as “NPC,” refers to the disorder as it is known in the medical art, and is distinct from Type A or B. NPC patients are not able to metabolize cholesterol and other lipids properly within the cell. Consequently, excessive amounts of cholesterol accumulate within the liver and spleen and excessive amounts of other lipids accumulate in the brain. NPC causes a secondary reduction of ASM (acid sphingomyelinase) activity such as is characteristic of Type A and B. Type C Niemann-Pick disease has an estimated 500 cases diagnosed worldwide. It is believed, however, that the number of people affected by NPC is much higher, but diagnostic difficulties do not allow an accurate assessment of the population indicence. NPC has been initially diagnosed as a learning disability, mild retardation, “clumsiness,” and delayed development of fine motor skills. It is not uncommon for a family to spend several years seeking a diagnosis before NPC is identified. NPC is always fatal. The majority of children with NPC die before age 20 (many die before the age of 10). Late onset of symptoms can lead to longer life spans, but it is extremely rare for any person with NPC to reach age 40. A recent study based on genomic analyses suggests the incidence of infantile onset NPC is 1:90,000, but when all forms are considered, including the adult onset variants, the disease may be as common as 1/19,000-1/36,000. There is currently no curative therapy for any form of NPC disease.

The term “NPC1” refers to the wildtype NPC1 gene or protein, various mutant forms of which are associated with Niemann-Pick Type C disease by leading to the accumulation of intracellular unesterified cholesterol. For convenience, the human gene is referred to as hNPC1 or NPC1 and the murine gene as mNPC1 or Npc1 (this same nomenclature is also used to distinguish between the human and murine cDNAs and proteins). Where no “h” or “m” designation is given, reference to the human NPC1 gene generally is intended. The definition of an NPC1 gene includes the various sequence polymorphisms that exist in the species in question, i.e., the term “hNPC1” or a wildtype hNPC1 encompasses all various sequence polymorphisms in humans.

The NPC1 protein or a derivative may be functionally characterized by its ability, when expressed in NPC cells, to correct the lysosomal cholesterol accumulation phenotype that is characteristic of such cells. Thus, “NPC1 protein biological activity” refers to the ability of a protein to correct the lysosomal cholesterol accumulation phenotype that is characteristic of NPC cells.

A “wildtype NPC1 protein” refers to any protein encoded by a wild-type gene that is capable of having normal (level of function absent disease or disorder) biological activity when expressed or introduced in vivo. Such functionality can be tested by any means known to establish functionality of a protein.

The term “NPC1 derivative gene,” which can include a “mutant NPC1 gene” or a codon optimized NPC1”, refers to any non-wildtype NPC1 sequence. Typically, a “mutant NPC1 gene” refers to a non-wildtype sequence that results in an aberrant functioning NPC1 protein, and thus, NPC disease. However, the term “NPC1 derivative gene” is meant to be broad enough to encompass an NPC1 mutant gene, but also any other NPC1 gene carrying a genetic change that may result an NPC1 protein having any of an increase, a decrease, or no change in activity as compared to the wildtype protein.

The term “NPC1 protein, derivative, or functional variant thereof,” which can include a “mutant NPC1 protein” or a “codon optimized NPC1”, refers to any non-wildtype NPC1 sequence or fragment thereof. Typically, a “mutant NPC1 protein” refers to a non-wildtype NPC1 polypeptide that has an aberrant function as compared to a wildtype NPC1 protein, and which results in NPC1 disease. However, the term “NPC1 protein, derivative, or functional variant thereof” is meant to be broad enough to encompass an NPC1 mutant protein, but also any other NPC1 protein carrying a genetic change (including a fragment) that may result an NPC1 protein having any of an increase, a decrease, or no change in activity as compared to the wildtype NPC1 protein. In the case of the present disclosure, the “NPC1 protein, derivative, or functional variant thereof” can also refer to homologous NPC1 proteins from non-human sources, e.g., mouse, monkey, horse, rabbit, and the like.

“Codon optimization” refers to the process of altering a naturally occurring polynucleotide sequence to enhance expression in the target organism, e.g., humans. As described herein, the human NPC1 gene can be altered to replace codons that occur less frequently in human genes with those that occur more frequently and/or with codons that are frequently found in highly expressed human genes. This method involves determining the relative frequency of a codon in the protein-encoding genes in the human genome. For example, isoleucine can be encoded by AUU, AUC, or AUA, but in the human genome, AUC (47%), AUU (36%), and AUA (17%) are variably used to encode isoleucine in proteins. Therefore, in the proper sequence context, AUA would be changed to AUC to allow this codon to be more efficiently translated in human cells. However, any known technique for codon optimization for expression may be utilized.

The term “NPC sufferer” or “NPC homozygote” refers to a person who carries a mutant NPC1 or NPC2 gene, such that the person exhibits clinical symptoms of Niemann-Pick type C disease.

The term “NPC carrier” or “NPC heterozygote” refers to a person who does not exhibit clinical symptoms of NPC, but who carries one mutant form of the NPC1 or NPC2 gene and may transmit this mutant gene to progeny.

As used herein, the term “cholesterol storage disease or disorder” is meant to refer to a disease or disorder of or related to cholesterol metabolism, optionally that is treatable via use of gene therapy for delivery of NPC to a subject. Exemplary “cholesterol storage disease or disorders” include but are not limited to Niemann-Pick disease, type C1. Whether cholesterol storage and related pathophysiology may be impacted by NPC1 function in other conditions is certain and extends the utility of NPC directed therapies, specifically NPC1 gene therapy, toward other more common disorders in the future. For example, a subset of neuropsychiatric disorders, such as dementia, seizures, and atherosclerotic brain disease might to be influenced by or improved after cholesterol reduction mediated by NPC1 activity and as such, these groups of patients might be candidates for NPC1 viral gene therapy.

### Terms Relating to Molecular Biology

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization; B. D. Hames & S. J. Higgins eds. (1985); Transcription And Translation; [B. D. Hames & S. J. Higgins, eds. (1984); Animal Cell Culture; R. I. Freshney, ed. (1986); Immobilized Cells And Enzymes; IRL Press, (1986); B. Perbal, A Practical Guide To Molecular Cloning (1984); F. M. Ausubel et al. (eds.), Current Protocols in Molecular Biology, John Wiley & Sons, Inc. (1994).

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein, may be associated with other proteins or nucleic acids, or both, with which it associates in the cell, or with cellular membranes if it is a membrane-associated protein. In a specific embodiment, an isolated NPC1 protein is a recombinant NPC1 protein expressed from an expression vector. An isolated material may be, but need not be, purified.

As used herein, the term “cDNA” (complementary DNA) refers to a piece of DNA lacking internal, non-coding segments (introns) and regulatory sequences that determine transcription. cDNA can be synthesized in the laboratory by reverse transcription from messenger RNA extracted from cells.

As used herein, the term “ORF” (open reading frame) refers to a series of nucleotide triplets (codons) coding for amino acids without any termination codons. These sequences are usually translatable into a peptide, such as _{co}NPC1 or a fusion protein comprising the same.

As used herein, the term “ortholog” refers to two nucleotide sequences that share a common ancestral sequence and diverged when a species carrying that ancestral sequence split into two species. Ortholgous sequences are also homologous sequences.

As used herein, the terms “probes” and “primers” refers to oligonucleotide sequences that may readily be prepared based on the nucleic acids provided by this disclosure. A probe comprises an isolated nucleic acid attached to a detectable label or reporter molecule. Typical labels include radioactive isotopes, ligands, chemiluminescent agents, and enzymes. Methods for labeling and guidance in the choice of labels appropriate for various purposes are discussed, e.g., in Sambrook et al. (1989) and Ausubel et al. (1987). “Primers” are short nucleic acids, preferably DNA oligonucleotides 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using probes and primers are described, for example, in Sambrook et al. (1989), Ausubel et al. (1987), and Innis et al., (1990). PCR primer pairs can be derived from a known sequence, for example, by using computer programs intended for that purpose such as Primer (Version 0.5, 1991, Whitehead Institute for Biomedical Research, Cambridge, Mass.). One of skill in the art will appreciate that the specificity of a particular probe or primer increases with its length. Thus, for example, a primer comprising 20 consecutive nucleotides of the human NPC1 cDNA or gene will anneal to a-target sequence such as an NPC1 gene homolog from rat contained within a genomic rat genomic DNA library with a higher specificity than a corresponding primer of only 15 nucleotides. Thus, in order to obtain greater specificity, probes and primers may be selected that comprise 20, 25, 30, 35, 40, 50 or more consecutive nucleotides of the NPC1 cDNA or gene sequences.

The present disclosure thus includes isolated nucleic acid molecules that comprise specified lengths of the disclosed NPC1 DNA (or cDNA) or gene sequences. Such molecules may comprise at least 20, 25, 30, 35, 40 or 50 consecutive nucleotides of these sequences and may be obtained from any region of the disclosed sequences.

As used herein, a “vector” nucleic acid molecule as introduced into a host cell, thereby producing a transformed host cell. A vector may include nucleic acid sequences that permit it to replicate in the host cell, such as an origin of replication. A vector may also include one or more selectable marker genes and other genetic elements known in the art. A vector may include a “gene transfer vector,” “gene therapy vector,” or “gene therapy construct,” or similar terms, which refer to specific vector constructs that are suitable to conduct gene transfer to administer a desired gene.

The terms “vector,” “cloning vector,” and “expression vector” mean the vehicle by which an ASM DNA or RNA sequence (e.g., a foreign gene) can be introduced into a host cell so as to transform the host and promote expression (e.g., transcription and translation) of the introduced sequence. Vectors include any genetic element, such as a plasmid, phage, transposon, cosmid, chromosome, virus, virion, etc., which is capable of replication when associated with the proper control elements and which can transfer ASM gene sequences between cells. Thus, the term includes cloning and expression vehicles, as well as viral vectors.

As used herein, the term “expression system” means a host cell and compatible vector under suitable conditions, e.g., for the expression of a _{co}NPC1 protein coded for by foreign DNA carried by the vector and introduced to the host cell. Common expression systems include E. coli host cells and plasmid vectors, insect host cells such as Sf9, Hi5 or S2 cells and Baculovirus vectors, and expression systems, and mammalian host cells and vectors. The term “expression system” also may refer to a suitable gene therapy vector, which may be delivered by any means, including ex vivo and in vivo methods.

The term “host cell” means any cell of any organism that is selected, modified, transformed, grown, or used or manipulated in any way, for the production of a substance by the cell, for example the expression by the cell of a human ASM gene, including a DNA or RNA sequence, or the NPC1 enzyme. Host cells can further be used for preliminary evaluation of other assays. A “recombinant DNA molecule” is a DNA molecule that has undergone a molecular biological manipulation or engineering. In one embodiment of the disclosure, the host cell is a fibroblast.

A “gene” is a sequence of nucleotides that code for a “gene product”. Generally, a gene product is a protein. However, a gene product can also be another type of molecule in a cell, such as an RNA (e.g., a tRNA or a rRNA). For the purposes of the present disclosure, a gene product also refers to an mRNA sequence which may be found in a cell. As used herein, a gene can refer to the nucleotide sequences encoding wild-type or mutant or codon-optimized NPC1 gene.

As used herein, a “transformed cell” is a cell into which has been introduced a nucleic acid molecule by molecular biology techniques or gene therapy techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration and includes both in vitro and in vivo conditions.

As used herein, the term “purified” does not require absolute purity; rather, it is intended as a relative term. Thus, for example, a purified NPC1 protein preparation is one in which the NPC1 protein is more pure than the protein in its natural environment within a cell. Preferably, a preparation of an NPC1 protein is purified such that the NPC1 protein represents at least 50% of the total protein content of the preparation.

As used herein, the term “operably linked” refers to where a first nucleic acid sequence (e.g., an NPC1 gene) is operably linked with a second nucleic acid sequence (e.g., a promoter sequence, protein transduction domain gene, and/or a tag, such as a 3xFLAG®-tag) when the first nucleic acid sequence is placed in a functional relationship with the second nucleic acid sequence. For instance, a promoter is operably linked to a coding sequence if the promoter affects the transcription or expression of the coding sequence and two genes are operably linked when they are expressed together to make a fusion protein. Generally, operably linked DNA sequences are contiguous and, where necessary to join two protein coding regions, in the same reading frame.

As used herein, the term “recombinant nucleic acid” is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

As used herein, the term “sequence identity” refers to the similarity between two nucleic acid sequences, or two amino acid sequences and is expressed in terms of the similarity between the sequences, otherwise referred to as sequence identity. Sequence identity is frequently measured in terms of percentage identity (or similarity or homology); the higher the percentage, the more similar the two sequences are. Homologs of the human and mouse NPC1 proteins will possess a relatively high degree of sequence identity when aligned using standard methods.

Methods of alignment of sequences for comparison are well-known in the art. Various programs and alignment algorithms are described in: Smith and Waterman (1981); Needleman and Wunsch (1970); Pearson and Lipman (1988); Higgins and Sharp (1988); Higgins and Sharp (1989); Corpet et al. (1988); Huang et al. (1992); and Pearson et al. (1994). Altschul et al. (1994) presents a detailed consideration of sequence alignment methods and homology calculations.

The NCBI Basic Local Alignment Search Tool (BLAST) (Altschul et al., 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI online site under the “BLAST” heading. A description of how to determine sequence identity using this program is available at the NCBI online site under the “BLAST overview” subheading.

Homologs of the disclosed NPC1 and NPC2 proteins are typically characterized by possession of at least 70% sequence identity counted over the full length alignment with the disclosed amino acid sequence of either the human or mouse NPC1/NPC2 sequences using the NCBI Blast 2.0, gapped blastp set to default parameters. Proteins with even greater similarity to the reference sequences will show increasing percentage identities when assessed by this method, such as at least 75%, at least 80%, at least 90% or at least 95% sequence identity. When less than the entire sequence is being compared for sequence identity, homologs will typically possess at least 75% sequence identity over short windows of 10-20 amino acids, and may possess sequence identities of at least 85% or at least 90% or 95% depending on their similarity to the reference sequence. Methods for determining sequence identity over such short windows are described at the NCBI online site under the “Frequently Asked Questions” subheading. One of skill in the art will appreciate that these sequence identity ranges are provided for guidance only; it is entirely possible that strongly significant homologs could be obtained that fall outside of the ranges provided. The present disclosure provides not only the peptide homologs are described above, but also nucleic acid molecules that encode such homologs, such as those generated by codon optimization. In an embodiment, changing the nucleotide sequence on the corresponding codons will generate a synthetic NPC1 or NPC2 gene that would have improved translation efficiency and detection in the presence of the endogenous gene.

One indication that two nucleic acid sequences are substantially identical is that the polypeptide which the first nucleic acid encodes is immunologically cross reactive with the polypeptide encoded by the second nucleic acid (e.g., a human NPC1 protein and an NPC1homolog from another species, or a variant human NPC1 protein).

Another indication that two nucleic acid sequences are substantially identical is that the two molecules hybridize to each other under stringent conditions. Stringent conditions are sequence dependent and are different under different environmental parameters. Generally, stringent conditions are selected to be about 5° C. to 20° C. lower than the thermal melting point (Tm) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al. (1989) and Tijssen (1993) and are otherwise known in the art.

### Terms Relating to Gene Therapy

The term “gene therapy” refers to a method of changing the expression of an endogenous gene by exogenous administration of a gene, i.e., a wildtype or mutant or codon-optimized NPC1 gene. As used herein, gene therapy also refers to the replacement of a defective NPC1 gene, or replacement of a missing NPC1 gene, by introducing a functional gene (e.g., codon-optimized gene) or portion of a gene corresponding to the defective or missing NPC1 gene into somatic or stem cells of an individual in need. Gene therapy can be accomplished by “ex vivo” methods, in which differentiated or somatic stem cells are removed from the individual's body followed by the introduction of a normal copy of the defective gene into the explanted cells using a viral vector as the gene delivery vehicle. In addition, in vivo transfer involves direct gene transfer into cells in the individual in situ using a broad range of viral vectors (e.g., AAV9 or Anc80), liposomes, exosomes, nanoparticles, protein: DNA complexes, modified nucleic acids or naked DNA in order to achieve a therapeutic outcome.

The term “transgene” refers to a polynucleotide that is introduced into a cell and is capable of being expressed under appropriate conditions and confers a desired property to a cell into which it was introduced, or otherwise leads to a desired therapeutic outcome.

The terms “genome particles (gp),” or “genome equivalents,” or genome copies (gc) as used in reference to a viral titer, refer to the number of virions containing the recombinant AAV DNA genome, regardless of infectivity or functionality. The number of genome particles in a particular vector preparation can be measured by procedures such as described elsewhere herein, or for example, in Clark et al. (1999) Hum. Gene Ther., 10:1031-1039; Veldwijk et al. (2002) Mol. Ther., 6:272-278.

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in Mclaughlin et al. (1988) J. Virol., 62:1963-1973.

The term “transducing unit (tu)” as used in reference to a viral titer, refers to the number of infectious recombinant AAV vector particles that result in the production of a functional transgene product as measured in functional assays such as described elsewhere herein, or for example, in Xiao et al. (1997) Exp. Neurobiol., 144:113-124; or in Fisher et al. (1996) J. Virol., 70:520-532 (LFU assay).

### Terms Relating to Therapeutic Application

The present disclosure further provides compositions and methods for ameliorating, treating, and/or preventing at least one symptom of a cholesterol storage disease or disorder, such as Niemann-Pick disease, type C caused by mutation or malfunction of the NPC1 enzyme which are encoded by the NPC1 gene.

The present disclosure relates to a particular set of newly developed expression constructs for the therapeutic expression of NPC1 for ameliorating, treating, and/or preventing at least one symptom of Niemann-Pick disease, type C (“NPC”) caused as a result of mutations to the NPC1 gene.

As used herein, the term “administering” is meant to refer to a means of providing the composition (e.g., to the subject in a manner that results in the composition being inside the subject's body. Such an administration can be by any route including, without limitation, subcutaneous, intradermal, intravenous, intra-arterial, intraperitoneal, sublingual, buccal, and intramuscular. In certain embodiments, the delivery may be appropriate for CNS delivery, e.g., epidural, intracerebral, or intracerebroventricular.

The present disclosure provides a number of compositions (e.g., sequences and vectors) that are useful for the development of highly specific drugs or biologics to treat or prevent a disease or disorder in a subject, as further characterized by the methods delineated herein. In addition, the methods of the present disclosure provide a facile means to identify therapies that are safe for use in subjects. Other disorders that can feature cholesterol storage are contemplated, including adult forms of dementia and conditions that may be caused, in part, by diminished activity of NPC1.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)).

A “subject” or “patient” is a human or an animal that has developed, or is likely to develop NPC disease, more particularly a mammal, preferably a rodent or a primate, and most preferably a human. In one embodiment, the patient is a member of the Ashkenazi Jewish population who has been diagnosed with, or who has been identified as having an increased risk of developing NPC disease due to inherited mutations in the NPC1 or NPC2 gene. In another embodiment, the patient is a member of the French Canadian population of Nova Scotia, an inhabitant of the Maghreb region (Tunisia, Morocco, Algeria) of North Africa, or a member of the Spanish-American population of southern New Mexico and Colorado. However, Niemann-Pick disease is pan-ethnic, and the term subject encompasses anyone in the world having, or genetically at risk of developing, NPC disease. The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

“Ameliorate”, “ameliorating”, “amelioration”, or the like, refers to decreasing, suppressing, attenuating, diminishing, arresting, or stabilizing the development or progression of a disease.

“Treatment”, or “treating” as used herein, is defined as the application or administration of a therapeutic agent (e.g., an AAV-coNPC1 vector) to a patient, or application or administration of a therapeutic agent to an isolated tissue or cell line from a patient, who has a disorder with the purpose to cure, heal, alleviate, relieve, alter, remedy, ameliorate, improve or affect the disease or disorder, or symptoms of the disease or disorder. The term “treatment” or “treating” is also used herein in the context of administering agents prophylactically. In the context of the present disclosure, the symptoms that may be alleviated can include, but are not limited to, the accumulation of sphingomyelin in reticuloendothelial lysosomes, which results in hepatosplenomegaly, psychomotor retardation, pulmonary abnormalities, progressive neurodegeneration. In some instances, treatment will prevent death resulting from NPC disease.

The term “prevention” refers to the prevention of the onset of the disease, which means to prophylactically interfere with a pathological mechanism that results in the disease. In the context of the present disclosure, such a pathological mechanism can be an increase expression of mutant NPC1 or NPC2.

The terms “effective dose” or “effective dosage” or “therapeutically effective amount” are defined as an amount sufficient to achieve or at least partially achieve the desired effect. The term “therapeutically effective dose” is defined as an amount sufficient to cure or at least partially arrest the disease and its complications in a patient already suffering from the disease or prevent the disease prophylactically. The term “patient” includes human and other mammalian subjects that receive either prophylactic or therapeutic treatment. As it pertains to the instant disclosure, the term “therapeutically effective amount” also is used herein to mean an amount or dose of a gene therapy vector encoding NPC1 (or a mutant or functional variant thereof) sufficient to increase the level of NPC1 activity over the mutant or defective level to about 3-5%, preferably by about 10%, and more preferably by about 30%, or about 40%, or about 50%, or about 60%, or about 70%, or about 80%, or about 90%, or about 95%, or even up to 100% of the level found in normal cells. Preferably, a therapeutically effective amount can ameliorate or prevent a clinically significant deficit in NPC1 in the subject. Alternatively, a therapeutically effective amount is sufficient to cause an improvement in a clinically significant condition/symptom in the subject, e.g., amelioration of progressive neurodegeneration in Type C NPD patients.

Certain methodologies of the instant disclosure include at least one step that involves comparing a value, level, feature, characteristic, property, etc. to a “suitable control”, referred to interchangeably herein as an “appropriate control”. A “suitable control” or “appropriate control” is a control or standard familiar to one of ordinary skill in the art useful for comparison purposes. In one embodiment, a “suitable control” or “appropriate control” is a value, level, feature, characteristic, property, etc. determined prior to performing a gene therapy methodology, as described herein. For example, a transcription rate, mRNA level, translation rate, protein level, biological activity, cellular characteristic or property, genotype, phenotype, etc. can be determined prior to introducing an AAV or other vector of the disclosure into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Other definitions appear in context throughout the disclosure.

### Gene Therapy Vectors

In one aspect, the present disclosure relates to gene therapy vectors or constructs comprising NPC1 genes, or derivatives and/or mutants thereof (e.g., NPC1 gene that is codon modified to enhance or improve NPC1 protein expression in a subject, such as a human), which are operably linked to at least a promoter element that is capable of being expressed in a tissue of the central nervous system.

In one embodiment, the present disclosure relates nucleic acid constructs for the expression of a therapeutic amount of NPC1 (e.g., _{co}NPC1) in a cell.

In another embodiment, the nucleic acid construct or gene therapy vector comprise a human codon-optimized NPC1 gene. For example, the nucleic acid construct or gene therapy vector can comprise a NPC1 codon optimized nucleic acid sequence selected from the group consisting of SEQ ID NOs: 1-8. For example, the nucleic acid construct or gene therapy vector can comprise a nucleic acid sequence selected from the group consisting of SEQ ID NOs: 1-8.

In another embodiment, the _{co}NPC1 is translationally fused to a protein transduction domain (PTD) gene (such as, HIV-tat, TP10, or TP2) to form a _{co}NPC1-PTD fusion protein. In other embodiments, the _{co}NPC1 is translationally fused to a tag (e.g., a 1x FLAG®-tag or a 3xFLAG®-tag) to form a NPC1-tag fusion protein (e.g., _{co}NPC1-1xFLAG® tag or _{co}NPC1-3xFLAG® tag). The _{co}NPC1 can be translationally fused to a PTD gene and a tag to form a _{co}NPC1-PTD-FLAG® tag or _{co}NPC1-FLAG® tag-PTD fusion protein, wherein the fusion protein is capable of cross-correcting non-transformed neighboring cells.

In an embodiment, at least one of the cell, the neighboring cells, or both, are neuronal cells.

In an embodiment, the NPC1 gene may be under the control of a promoter, wherein the promoter is a truncated EF1 promoter (EF1ₜ promoter) or a mini EF1 promoter (EF1α promoter), or the EF1 short promoter (EF1α s).

In an embodiment, the nucleic acid construct comprises an expression cassette comprising a nucleotide sequence selected from the group consisting of SEQ ID NOs: 26-38.

In an embodiment, the gene therapy vector may comprise shortened or full-length, wild type AAV inverted terminal repeats (ITRs) that flank the transgene. In an embodiment, besides the ITRs, the nucleic acid construct may be devoid of any other sequence from the wild-type AAV genome.

In an embodiment, the shortened or full-length AAV ITRs are AAV2 ITRs. In a particular embodiment, the shortened or full-length AAV ITRs each comprise the nucleotide sequence of SEQ ID NO: 48. In another particular embodiment, the shortened or full-length AAV ITRs each comprise the nucleotide sequence of SEQ ID NO: 49.

In an embodiment, the _{co}NPC1 protein, the _{co}NPC1-PTD fusion protein and NPC1-tag fusion protein can be included in a vector, more particularly, in an expression cassette, with a synthetic intron sequence (SEQ ID NO: 25) which may enhance expression by providing more effective splicing of the transgene as compared to a vector which does not comprise the synthetic intron sequence.

In an embodiment, the NPC1 gene is under the control of a promoter, and the Intron S is positioned between the promoter and the NPC1 gene. In a particular embodiment, the Intron S comprises the nucleotide sequence of SEQ ID NO: 25.

In an embodiment, the nucleic acid construct may further comprise a nucleotide sequence encoding an antibiotic resistance marker. In an embodiment, the antibiotic resistance marker is an ampicillin resistance marker. In another embodiment, the antibiotic resistance marker is a kanamycin resistance marker.

In an embodiment, the nucleic acid construct comprises an expression vector which is capable of expressing the NPC1 protein, the NPC1-PTD fusion protein, or the NPC1 fusion protein, optionally comprising a tag moiety.

In an embodiment, the invention comprises a cell comprising the expression vector, wherein the cell produces the expression vector and/or the cell expresses the NPC1 protein.

In an embodiment, the invention comprises a method of ameliorating, treating, or preventing Niemann-Pick disease, type C1 in a subject, the method comprising administering a therapeutically effective amount of an expression vector to at least one cell, the expression vector comprising a nucleic acid molecule comprising a human codon-optimized NPC1 gene, wherein the expression vector is effective at of ameliorating, treating, or preventing at least one symptom of Niemann-Pick disease, type C1 in the subject.

In an embodiment, the NPC1 of the inventive method is translationally fused to a PTD to form a NPC1-PTD fusion protein, wherein the NPC1-PTD fusion protein is capable of cross-correcting non-transformed neighboring cells. In particular embodiments, the PTD protein transduction domain may be HIV-Tat, TP10, or TP2.

In an embodiment, the NPC1 gene of the inventive method is under the control of a promoter. In embodiments, the promoter may be a truncated EF1α promoter (EF1αₜ promoter) or a mini EF1α promoter (EF1α promoter), or the short EF1α promoter (EF1α S).

In an embodiment, the NPC1 gene of the inventive method may be translationally fused to a tag moiety to form a NPC1-tag fusion protein, a NPC1-PTD-tag fusion protein, or a NPC1-tag-PTD fusion protein.

In an embodiment, the nucleic acid molecule of the inventive method comprises a nucleic acid sequence selected from the group consisting of SEQ ID NO: 1-8.

In an embodiment, the expression vector of the inventive method may comprise an expression cassette, with a synthetic intron sequence (SEQ ID NO: 25), which sequence may enhance expression by providing more effective splicing of the transgene as compared to the gene therapy vector which does not comprise the synthetic intron sequence. In an embodiment, the NPC1 gene of the inventive method is under the control of a promoter, and the Intron S is positioned between the promoter and the NPC1 gene. In a particular embodiment, the Intron S comprises the nucleotide sequence of SEQ ID NO: 25.

In a further embodiment, the inventive method comprises a nucleic acid construct comprising an expression cassette comprising a nucleotide sequence selected from the group consisting of SEQ ID NOs: 26-38.

In an embodiment, the expression vector of the inventive method may comprise first and second full-length, wild type AAV inverted terminal repeats (ITRs) that flank the transgene. In an embodiment, besides the ITRs, the nucleic acid construct may be devoid of any other sequence from the wild-type AAV genome. In an embodiment, the first and second AAV ITRs are AAV2 ITRs. In a particular embodiment, the first and second AAV ITRs each comprise the nucleotide sequence of SEQ ID NO: 48. In another particular embodiment, the first and second AAV ITRs each comprise the nucleotide sequence of SEQ ID NO: 49.

In an embodiment, inventive method may further comprise a nucleotide sequence encoding an antibiotic resistance marker. In an embodiment, the antibiotic resistance marker is an ampicillin resistance marker. In another embodiment, the antibiotic resistance marker is a kanamycin resistance marker.

In an embodiment, the nucleic acid construct may comprise first and second AAV ITRs, the human codon-optimized NPC1 gene, and one or more of a nucleotide sequence encoding a PTD, a promoter, an Intron S, and a nucleotide sequence encoding a tag, wherein the NPC1 gene and all of the one or more of the nucleotide sequence encoding the PTD, the promoter, and the nucleotide sequence encoding the tag are positioned between the first and second AAV ITRs. In another embodiment, the nucleic acid construct may comprise first and second AAV ITRs, the human codon-optimized NPC1 gene, and all of a nucleotide sequence encoding a PTD, a promoter, an Intron S, and a nucleotide sequence encoding a tag, wherein the NPC1 gene and all of the one or more of the nucleotide sequence encoding the PTD, the promoter, and the nucleotide sequence encoding the tag are positioned between the first and second AAV ITRs.

In one embodiment, the fusion protein is capable of cross-correcting non-transformed neighboring cells, such as neuronal cells.

In certain embodiments, the gene therapy vectors or constructs comprise an NPC1 gene, or a derivative and/or mutant NPC1 gene. The NPC1 gene, including any derivatives and/or mutants thereof, can encode a codon optimized NPC1 polypeptide, as described herein, as well as any functional fragment or variants thereof. The variants or functional fragments of the codon optimized NPC1 may have increased or decreased activity as compared to a wildtype NPC1 protein, or the activity may be unchanged.

The NPC1 nucleotide sequences comprising a coding region for NPC1 proteins may be obtained from any source, including human, mouse, horse, pig, monkey, and the like. The nucleotide sequences encoding NPC1, and NPC1 homologs from species other than human, are generally known in the art and can be obtained from public sequence repositories, including, for example, GenBank. In particular, cDNA sequences encoding NPC1 proteins (or variants thereof) may also be obtained from public sequence repositories such as GenBank.

For example, the following NPC1 sequences (or any variants comprising or genetically modified to comprise any mutations that encode a functional variant NPC1) that have been codon optimized for expression in humans are contemplated for use in the gene therapy constructs of the present disclosure:
GenBank Accession No. BC063302 (Homo sapiens Niemann-Pick disease, type C1, mRNA (cDNA clone), which provides the NPC1 cDNA coding sequence SEQ ID NO: 9) and the NPC1 polypeptide amino acid sequence (SEQ ID NO: 41);
GenBank Accession No. BC117178 (Homo sapiens NPC1 (Niemann-Pick disease, type C1, gene)-like 1 mRNA (cDNA clone), which provides a variant NPC1 cDNA coding sequence (SEQ ID NO: 11) and NPC1 polypeptide amino sequence (SEQ ID NO: 42);
GenBank Accession No. BC143756 (Homo sapiens NPC1 (Niemann-Pick disease, type C1, gene)-like 1, mRNA (cDNA clone), which provides a variant NPC1 cDNA coding sequence (SEQ ID NO: 12) and NPC1 polypeptide amino sequence (SEQ ID NO: 43);
GenBank Accession No. AF258783.1 (Felis catus Niemann-Pick type C1 disease protein (NPC1) mRNA, complete cds) which provides cat NPC1 cDNA coding sequence (SEQ ID NO: 13) and cat NPC1 polypeptide amino sequence (SEQ ID NO: 44);
GenBank Accession No. BC054539 (Mouse Npc1 (Niemann-Pick disease, type C1, gene) mRNA (cDNA clone), which provides mouse Npc1 cDNA coding sequence and mouse NPC1 polypeptide amino sequence (SEQ ID NO: 45);
GenBank Accession No. BC151276 (Bovine NPC1 (Niemann-Pick disease, type C1, gene) mRNA (cDNA clone), which provides bovine NPC1 cDNA coding sequence (SEQ ID NO: 15) and bovine NPC1 polypeptide amino sequence (SEQ ID NO: 46); and
GenBank Accession No. BC090541 (Zebrafish NPC1 (Niemann-Pick disease, type C1, gene) mRNA (cDNA clone), which provides Zebrafish NPC1 cDNA coding sequence (SEQ ID NO: 16) and Zebrafish NPC1 polypeptide amino sequence (SEQ ID NO: 47).

The disclosed subject matter further encompasses any NPC1 gene and/or polypeptide sequence not expressly indicated here, but which is publicly available at the time of the present disclosure, or which becomes available after the time of the disclosure.

In preferred embodiments, the transgene encodes a biologically active molecule, expression of which in the subject, e.g., in the CNS of a subject, results in at least partial correction of the cholesterol storage disease or disorder, for example, Niemann-Pick disease, Type C. In some embodiments, the transgene encodes NPC1 (or a functional variant and/or fragment thereof) that has been codon optimized for expression in a subject, such as a human. The genomic and functional mRNA, cDNA and corresponding polypeptide sequences of human, mouse, or other species NPC1 genes and proteins are known, as indicated above, and in particular are available as GenBank Accession Nos.: NM_000271.4 (SEQ ID NO: 17); NM_008720.2 (SEQ ID NO: 18); NM_006432.3 (SEQ ID NO: 19); NM_023409.4 (SEQ ID NO: 20); and corresponding polypeptides NP_000262.2 (SEQ ID NO: 21); NP_032746.2 (SEQ ID NO: 22); NP_006423.1 (SEQ ID NO: 23); and NP_075898.1 (SEQ ID NO: 24).

Nucleotide sequences encoding NPC1 genes (or variants thereof) may be obtained by any known molecular biology technique, including by cloning, synthesis, or PCR amplification. Oligonucleotides for using in amplification reactions and/or probes for use in gene cloning may be synthesized or otherwise obtained by any known means and based on the nucleotide sequences that flank the desired gene or coding region encoding the NPC1 target genes. Methods and techniques for gene cloning and/or PCR amplification are well known in the art and are discussed elsewhere herein.

The gene therapy constructs described herein also comprise a vector (or gene therapy expression vector) into which the gene of interest (e.g., codon optimized NPC1) is cloned or otherwise which includes the gene of interest in a manner such that the nucleotide sequences of the vector allow for the expression (constitutive or otherwise regulated in some manner) of the gene of interest with or without a PTD gene and/or a tag (e.g., FLAG®-tag). The vector constructs herein described include any suitable gene expression vector that is capable of being delivered to a tissue of interest (e.g., CNS) and which will provide for the expression of the gene of interest in the selected tissue of interest (e.g., CNS). In a preferred embodiment, the gene therapy vector is capable of efficient delivery to a tissue of the central nervous system, including the spine and the brain, and in particular, is capable of crossing the blood-brain barrier of the brain.

In a preferred embodiment, the vector is an adeno-associated virus (AAV) vector because of the capacity of AAV vectors to cross the blood-brain barrier and transduction of neuronal tissue. In the methods disclosed herein, AAV of any serotype can be used, though in certain embodiments, it is advantageous to use a vector that is capable of undergoing retrograde axonal transport in a disease-compromised brain. The serotype of the viral vector used in certain embodiments of the disclosure is selected from the group consisting of AAV1, AAV2, AAV3, AAV4, AAV5, AAV6, AAV7, AAV8, AAV9, AAVrh8, AAVrh10, AAVrh33, AAVrh34, AAV Anc80, AAV PHP.B, and others (see, e.g., Gao et al. (2002) PNAS, 99:11854-11859; and Viral Vectors for Gene Therapy: Methods and Protocols, ed. Machida, Humana Press, 2003, incorporated herein by reference). Other serotype besides those listed herein are also contemplated. In certain exemplary embodiments, AAV 2/9 is used. In a particular embodiment AAV2/Anc80 or AAV2/PHP.B is used. The herein disclosed compositions and methods may also use AAV chimeric vectors, whereby portions of AAV are fused with other similar vectors, such as Adenovirus.

AAV vectors are derived from single-stranded (ss) DNA parvoviruses that are nonpathogenic for mammals (reviewed in Muzyscka (1992) Curr. Top. Microb. Immunol., 158:97-129, incorporated herein by reference). Briefly, AAV-based vectors have the rep and cap viral genes that account for 96% of the viral genome removed, leaving the two flanking 145-basepair (bp) inverted terminal repeats (ITRs), which are used to initiate viral DNA replication, packaging and integration. In the absence of helper virus, wild-type AAV integrates into the human host-cell genome with preferential site-specificity at chromosome 19q 13.3 or it may remain expressed episomally. A single AAV particle can accommodate up to 5 kb of ssDNA, therefore leaving about 4.5 kb for a transgene and regulatory elements, which is typically sufficient. However, trans-splicing systems as described, for example, in U.S. Pat. No. 6,544,785, may nearly double this limit.

In an illustrative embodiment, the AAV backbone, comprising sequences between two AAV inverted terminal repeats (ITRs), is pseudotyped using the serotype 2 capsid to create an AAV2 vector. Adeno-associated virus of many serotypes, especially AAV2, have been extensively studied and characterized as gene therapy vectors. Those skilled in the art will be familiar with the preparation of functional AAV-based gene therapy vectors. Numerous references to various methods of AAV production, purification and preparation for administration to human subjects can be found in the extensive body of published literature (see, e.g., Viral Vectors for Gene Therapy: Methods and Protocols, ed. Machida, Humana Press, 2003, incorporated herein by reference). Additionally, AAV-based gene therapy targeted to cells of the CNS has been described in U.S. Pat. Nos. 6,180,613 and 6,503,888 (each of which are incorporated herein by reference).

Optionally, the AAV viral capsid is AAV2, AAV9, AAVrh8, AAVrh10, AAV Anc80, or AAV PHP.B.; however, the serotype of the viral capsid used in certain embodiments of the disclosure can be selected from among known viral capsids, including AAV viral capsids of other known serotypes.

Optionally, the gene therapy vector, e.g., AAV or AAV-based vector, can be modified to improve virus uptake into the target tissue of interest (e.g., CNS), viral stability, and tropism. For example, the capsid of an AAV vector may be modified with a ligand (e.g., synthetic or naturally occurring small molecule, peptide, or polypeptide, or other biomolecule) that binds to a receptor at or in the tissue of interest (e.g., CNS). Other modifications are possible to improve and/or enhance the functional properties of the vector being used to both target the tissue of interest and allow the construct to enter and effectively transduce the target cells. Such modifications will be within the skill set of a person having ordinary skill in the art.

Further information regarding the use of AAV vectors can be found in the art, for example, in Kaplitt et al. (1994) Nat. Genet., 8:148-154; Bartlett et al. (1998) Hum. Gene Ther., 9:1181-1186; and Passini et al. (2002) J. Neurosci., 22:6437-6446, each of which are incorporated herein by reference. Furthermore, these viral vectors can transduce a variety of CNS cell types, including neurons, when delivered by the systemic route, intrathecal route or by direct brain injection.

As further contemplated herein, the gene therapy vectors may comprise a transgene (e.g., codon optimized NPC1) that is operably linked to a promoter or other genetic transcriptional and/or translational control elements. Certain AAV vectors pre-engineered with or comprising promoters can be obtained from public sources, including, for example the websites for Vector Laboratories or Addgene.

In certain embodiments, the promoter is a promoter which is capable of efficient inducible expression in the CNS. In still other embodiments, the promoter is constitutively active in the CNS. In certain preferred embodiments, the promoter provides for selective expression in the CNS, and expression outside of the CNS is limited or entirely absent. Promoter sequences having differing characteristics and expression profiles are well known in the art, including those that are tissue-specific, tissue-non-specific, constitutive, and inducible. Reference can be further made to, for example, Papadakis et al., “Promoters and Control Elements: Designing Expression Cassettes for Gene Therapy,” Current Gene Therapy, 2004, 4, 89-113, the contents of which are incorporated herein by reference. Promoters contemplated by the present disclosure include, but are not limited to: Apo A-I, ApoE, serpina (TBG), alpha-1-antitrypsin (hAAT) (liver specific); MCK (muscle specific); GFAP, NSE, Synapsin I, Preproenkephalin, Dopamine b-hydroxylase (dbH), Prolactin, Myelin basic protein (neuronal-specific), GUSB, CBA, CAG and Ankyrin (erythroid specific).

In a particular embodiment, the disclosed compositions and methods utilize an AAV vectors that contain new NPC1 alleles that have been codon optimized to maximize human expression (_{co}NPC1).

In another embodiment, the NPC1 alleles may be tagged.

In another embodiment, the NPC1 alleles may be configured with at least one of one or more tags, transduction domain peptides in order to allow trans-cellular correction or with only the native protein transduction domain (PTD) peptides, or a combination thereof.

In another embodiment, the gene therapy vector may comprise a truncated EF1α promoter (EF1αₜ promoter), or a mini EF1α promoter (EF1α promoter), or the short EF1α promoter (EF1α S). For example, the coNPC1 or fusion protein may be operationally linked to a EF1αₜ promoter. The EF1αₜ promoter may be a truncated EF1 promoter, which may be operationally linked to a stronger translation initiation site at the 5′ end of the operationally linked nucleic acid, such as the codon optimized NPC1 alone or in the context of a fusion protein, as discussed herein. The gene therapy vector may also include multiple stop signals, which ensures maximal NPC1 gene expression and protein production in the cell.

In an embodiment, at least one of the cell, the neighboring cells, or both, are neuronal cells.

In an embodiment, the nucleic acid construct comprises an expression cassette comprising a nucleotide sequence selected from the group consisting of SEQ ID NOs: 26-38.

In an embodiment, the gene therapy vector may comprise first and second full-length, wild type AAV inverted terminal repeats (ITRs) that flank the transgene. In an embodiment, besides the ITRs, the nucleic acid construct may be devoid of any other sequence from the wild-type AAV genome.

In an embodiment, the first and second AAV ITRs are AAV2 ITRs. In a particular embodiment, the first and second AAV ITRs each comprise the nucleotide sequence of SEQ ID NO: 48. In another particular embodiment, the first and second AAV ITRs each comprise the nucleotide sequence of SEQ ID NO: 49.

In an embodiment, the _{co}NPC1 protein, the _{co}NPC1-PTD fusion protein and NPC1-tag fusion protein can be included in a vector, more particularly, in an expression cassette, with a synthetic intron sequence (SEQ ID NO: 25) which may enhance expression by providing more effective splicing of the transgene as compared to the vector which does not comprise the synthetic intron sequence.

In an embodiment, the NPC1 gene is under the control of a promoter, and the Intron S is positioned between the promoter and the NPC1 gene. In a particular embodiment, the Intron S comprises the nucleotide sequence of SEQ ID NO: 25.

In an embodiment, the nucleic acid construct may further comprise a nucleotide sequence encoding an antibiotic resistance marker. In an embodiment, the antibiotic resistance marker is an ampicillin resistance marker. In another embodiment, the antibiotic resistance marker is a kanamycin resistance marker.

In an embodiment, the nucleic acid construct comprises an expression vector which is capable of expressing the NPC1 protein, the NPC1-PTD fusion protein, or the NPC1 fusion protein, optionally comprising a tag moiety.

In an embodiment, the invention comprises a cell comprising the expression vector, wherein the cell produces the expression vector and/or the cell expresses the NPC1 protein.

In an embodiment, the invention comprises a method of ameliorating, treating, or preventing Niemann-Pick disease, type C1 in a subject, the method comprising administering a therapeutically effective amount of an expression vector to at least one cell, the expression vector comprising a nucleic acid molecule comprising a human codon-optimized NPC1 gene, wherein the expression vector is effective at of ameliorating, treating, or preventing at least one symptom of Niemann-Pick disease, type C1 in the subject.

In an embodiment, the NPC1 of the inventive method is translationally fused to a PTD to form a NPC1-PTD fusion protein, wherein the NPC1-PTD fusion protein is capable of cross-correcting non-transformed neighboring cells. In particular embodiments, the PTD protein transduction domain may be HIV-Tat, TP10, or TP2.

In an embodiment, the NPC1 gene of the inventive method is under the control of a promoter. In embodiments, the promoter may be may be a truncated EF1α promoter (EF1αₜ promoter) or a mini EF1α promoter (EF1α promoter) or the short EF1α promoter (EF1α S).

In an embodiment, the NPC1 gene of the inventive method may be translationally fused to a tag moiety to form a NPC1-tag fusion protein, a NPC1-PTD-tag fusion protein, or a NPC1-tag-PTD fusion protein.

In an embodiment, the nucleic acid molecule of the inventive method comprises a nucleic acid sequence selected from the group consisting of SEQ ID NO: 1-8.

In an embodiment, the expression vector of the inventive method may comprise an expression cassette, with a synthetic intron sequence (SEQ ID NO: 25), which sequence may enhance expression by providing more effective splicing of the transgene as compared to the gene therapy vector which does not comprise the synthetic intron sequence. In an embodiment, the NPC1 gene of the inventive method is under the control of a promoter, and the Intron S is positioned between the promoter and the NPC1 gene. In a particular embodiment, the Intron S comprises the nucleotide sequence of SEQ ID NO: 25.

In a further embodiment, the inventive method comprises a nucleic acid construct comprising an expression cassette comprising a nucleotide sequence selected from the group consisting of SEQ ID NOs: 26-38.

In an embodiment, the expression vector of the inventive method may comprise first and second full-length, wild type AAV inverted terminal repeats (ITRs) that flank the transgene. In an embodiment, besides the ITRs, the nucleic acid construct may be devoid of any other sequence from the wild-type AAV genome. In an embodiment, the first and second AAV ITRs are AAV2 ITRs. In a particular embodiment, the first and second AAV ITRs each comprise the nucleotide sequence of SEQ ID NO: 48. In another particular embodiment, the first and second AAV ITRs each comprise the nucleotide sequence of SEQ ID NO: 49.

In an embodiment, inventive method may further comprise a nucleotide sequence encoding an antibiotic resistance marker. In an embodiment, the antibiotic resistance marker is an ampicillin resistance marker. In another embodiment, the antibiotic resistance marker is a kanamycin resistance marker.

In an embodiment, the nucleic acid construct may comprise first and second AAV ITRs, the human codon-optimized NPC1 gene, and one or more of a nucleotide sequence encoding a PTD, a promoter, an Intron S, and a nucleotide sequence encoding a tag, wherein the NPC1 gene and all of the one or more of the nucleotide sequence encoding the PTD, the promoter, and the nucleotide sequence encoding the tag are positioned between the first and second AAV ITRs. In another embodiment, the nucleic acid construct may comprise first and second AAV ITRs, the human codon-optimized NPC1 gene, and all of a nucleotide sequence encoding a PTD, a promoter, an Intron S, and a nucleotide sequence encoding a tag, wherein the NPC1 gene and all of the one or more of the nucleotide sequence encoding the PTD, the promoter, and the nucleotide sequence encoding the tag are positioned between the first and second AAV ITRs.

### Methods of Treatment, Prevention, and/or Amelioration

In one aspect, the present disclosure provides methods for ameliorating, treating and/or preventing at least one symptom of a cholesterol storage disease or disorder in mammals, such as Niemann-Pick disease, Type C. In preferred embodiments, the populations treated by the methods of the present disclosure include, but are not limited to, patients having or at risk for developing a cholesterol storage disease or disorder, e.g., Niemann-Pick disease, type C1,particularly, if such a disease affects the CNS. In an illustrative embodiment, the disease is Niemann-Pick disease, Type C1.

In certain aspects of the disclosure, the method of treating, ameliorating and/or preventing a cholesterol storage disease or disorder comprises administration of a high titer gene therapy vector described herein (e.g., an AAV-based gene therapy vector) carrying a therapeutic transgene so that the transgene product is expressed at a therapeutic level in the CNS of a subject. In some embodiments, the viral titer of the composition is at least: (a) 5, 6, 7, 8, 8.4, 9, 9.3, 10, 15, 20, 25, or 50×10¹² gc/ml; (b) 5, 6, 7, 8, 8.4, 9, 9.3, 10, 15, 20, 25, or 50×10⁹ tu/ml; or (c) 5, 6, 7, 8, 8.4, 9, 9.3, 10, 15, 20, 25, or 50×10¹⁰ iu/ml. In further embodiments, the administration is accomplished by direct intraparenchymal injection of solution comprising a high titer gene therapy vector described herein (e.g., an AAV-based gene therapy vector) into the diseased brain, thereafter the transgene is expressed distally, contralaterally or ipsilaterally, to the administration site at a therapeutic level at least 2, 3, 5, 8 10, 15, 20, 25, 30, 35, 40, 45, or 50 mm from the administration site.

In further embodiments, the administration is accomplished by direct intrathecal injection of a solution comprising a high titer gene therapy vector described herein (e.g., an AAV-based gene therapy vector) into the spinal fluid compartment, as is routine for practioners of the art, and thereafter the transgene is expressed distally, contralaterally, ipsilaterally and globally in the CNS, to the administration site at a therapeutic level at least 2, 3, 5, 8 10, 15, 20, 25, 30, 35, 40, 45, or 50 mm from the administration site. In other embodiments, the administration is accomplished by direct intraventricular injection of a solution (which is routine for practitioners in the art) comprising the gene therapy vector described herein (e.g., a high titer of the gene therapy vector described herein). The administered gene therapy vector is expressed at least one of distally, contralaterally, ipsilaterally, globally, or a combination thereof, in the CNS.

In certain embodiments, the transgene product (e.g., NPC1 polypeptide) is expressed at a therapeutic level in a second site within the CNS distal to the first site. The distance between the first and the second sites is defined as the minimal distance region between the site of administration (first site) and the boundary of the detectable transduction of the distal site (second site) as measured using procedures known in the art, e.g., magnetic resonance imaging including spectroscopy or direct brain biopsy. Some neurons in the CNS of larger mammals may span large distances by virtue of their axonal projections. For example, in humans, some axons may span a distance of 1000 mm or greater. Thus, in various methods of the disclosure, a gene therapy vector of the disclosure can be axonally transported along the entire length of the axon at such a distance to reach and transduce the parent cell body.

A site of vector administration within the CNS can be chosen based on the desired target region of neuropathology and, optionally, the topology of brain circuits involved when an administration site and the target region have axonal connections. In certain embodiments, the target region can be defined, for example, using 3-D sterotaxic coordinates. In some embodiments, the administration site is chosen so that at least 0.1, 0.5, 1, 5, or 10% of the total amount of vector injected is delivered distally at the target region of at least 1, 200, 500, or 1000 mm³. An administration site may be localized in a region innervated by projection neurons connecting distal regions of the brain. For example, the substantia nigra and ventral tegmental area send dense projections to the caudate and putamen (collectively known as the striatum). Neurons within the substantia nigra and ventral tegmentum can be targeted for transduction by retrograde transport of a gene therapy construct described herein (e.g., AAV based vector) following injection into the striatum. As another example, the hippocampus receives well-defined, predictable axonal projections from other regions of the brain. Other administration sites may be localized, for example, in the spinal cord, brainstem (medulla and pons), mesencephalon, cerebellum, diencephalon (thalamus, hypothalamus), telencephalon (corpus striatum, cerebral cortex, or, within the cortex, the occipital, temporal, parietal or frontal lobes), or combinations thereof.

For identification of structures in the human brain, see, e.g., The Human Brain: Surface, Three-Dimensional Sectional Anatomy With MRI, and Blood Supply, 2nd ed., eds. Deuteron et al., Springer Vela, 1999; Atlas of the Human Brain, eds. Mai et al., Academic Press; 1997; and Co-Planar Sterotaxic Atlas of the Human Brain: 3-Dimensional Proportional System: An Approach to Cerebral Imaging, eds. Tamarack et al., Thyme Medical Pub., 1988. For identification of structures in the mouse brain, see, e.g., The Mouse Brain in Sterotaxic Coordinates, 2nd ed., Academic Press, 2000. If desired, the human brain structure can be correlated to similar structures in the brain of another mammal. For example, most mammals, including humans and rodents, show a similar topographical organization of the entorhinal-hippocampus projections, with neurons in the lateral part of both the lateral and medial entorhinal cortex projecting to the dorsal part or septal pole of the hippocampus, whereas the projection to the ventral hippocampus originates primarily from neurons in medial parts of the entorhinal cortex (Principles of Neural Science, 4th ed., eds Kandel et al., McGraw-Hill, 1991; The Rat Nervous System, 2nd ed., ed. Paxinos, Academic Press, 1995). Furthermore, layer II cells of the entorhinal cortex project to the dentate gyrus, and they terminate in the outer two-thirds of the molecular layer of the dentate gyrus. The axons from layer III cells project bilaterally to the cornu ammonis areas CA1 and CA3 of the hippocampus, terminating in the stratum lacunose molecular layer.

In certain embodiments, the target site can be located any region of the CNS, including the brain and the spinal cord, that contains neurons that project to the first (administration) site. In some embodiments, the second site is in a region of the CNS chosen from the substantia nigra, the medulla oblongata, or the spinal cord.

To deliver a gene therapy vector described herein specifically to a particular region of the central nervous system, especially to a particular region of the brain, it may be administered by sterotaxic microinjection. For example, on the day of surgery, patients will have the sterotaxic frame base fixed in place (screwed into the skull). The brain with sterotaxic frame base (MRI-compatible with fiduciary markings) will be imaged using high resolution MRI. The MRI images will then be transferred to a computer that runs stereotaxic software. A series of coronal, sagittal and axial images will be used to determine the target site of vector injection, and trajectory. The software directly translates the trajectory into 3-dimensional coordinates appropriate for the stereotaxic frame. Burr holes are drilled above the entry site and the stereotaxic apparatus localized with the needle implanted at the given depth. The vector in a pharmaceutically acceptable carrier will then be injected. The AAV vector is then administrated by direct injection to the primary target site and retrogradely transported to distal target sites via axons. Additional routes of administration may be used, e.g., superficial cortical application under direct visualization, or other non-stereotaxic application.

Optionally, non-CNS delivery can also be performed, e.g., for cholesterol storage diseases or disorders where non-CNS delivery would also be desirable. Such non-CNS delivery of the compositions (e.g., constructs) of the instant disclosure can be performed in addition to or as an alternative to CNS delivery. In certain such embodiments, injection, e.g., intravenous, intraperitoneal, etc. injection can be performed using the compositions of the instant disclosure. Direct delivery to large peripheral nerves is also considered.

In yet another method, a suitable AAV vector configured to express NPC1 can be encapsidated with a capsid known to afford transduction of the blood brain barrier and further penetration of the CNS and its elements. In this embodiment, the AAV vector can be delivered systemically, by IV infusion, and engender both peripheral and CNS correction, and depending upon the promoter and serotype of the vector, may enable neurovisceral correction.

The total volume of material to be administered, and the total number of vector particles to be administered, will be determined by those skilled in the art based upon known aspects of gene therapy. Therapeutic effectiveness and safety can be tested in an appropriate animal model. For example, for NPC, in any Npc1^{-/-} model mouse such as the Npc^{nih} homozygous mice.

In experimental mice, the total volume of injected vector, e.g., AAV vector, solution is, for example, between 1 to 10 μl. For other mammals, including the human brain, volumes and delivery rates are appropriately scaled. For example, it has been demonstrated that volumes of 150 μl can be safely injected in the primate brain (Janson et al. (2002) Hum. Gene Ther., 13:1391-1412). Treatment may consist of a single injection per target site, or may be repeated along the injection tract, if necessary. Multiple injection sites can be used. For example, in some embodiments, in addition to the first administration site, a composition comprising a gene therapy vector described herein carrying a transgene is administered to another site that can be contralateral or ipsilateral to the first administration site.

In another aspect, the disclosure provides a method of delivering a transgene product to a target cell of the CNS, which is a neuron or a glial cell, in a mammal afflicted with a cholesterol storage disease or disorder, e.g., Niemann-Pick disease, type C. The method comprises contacting an axonal ending of a neuron with a composition of the present disclosure (a nucleic acid construct or gene therapy vector comprising a codon optimized NCP1); allowing the viral particles to be endocytosed and retrogradely transported intracellularly along the axon to the nucleus of the neuron; allowing the transgene product to be expressed and transported within the membrane(s) of the neuron, wherein the transgene product thereby alleviates pathology related to cholesterol storage. In another embodiment, a gene therapy vector encoding a codon optimized NPC1, optionally with tags and/or a PTD, can be infused into a reservoir, such as an Omoyaya reservoir, for direct intracranial delivery. In some embodiments, the concentration of the AAV vector in the composition is at least: (a) 5, 6, 7, 8, 8.4, 9, 9.3, 10, 15, 20, 25, or 50×10¹² gc/ml; (b) 5, 6, 7, 8, 8.4, 9, 9.3, 10, 15, 20, 25, or 50×10⁹ tu/ml; or (c) 5, 6, 7, 8, 8.4, 9, 9.3, 10, 15, 20, 25, or 50×10¹⁰ iu/ml.

The present disclosure provides for both prophylactic and therapeutic methods of treating a subject at risk of (or susceptible to) a disease or disorder caused, in whole or in part, by altered cholesterol storage, optionally treatable via selective or systemic delivery of a NPC1-containing gene therapy vector to a subject.

In certain aspects, the disclosure provides a method for preventing in a subject, a disease or disorder as described herein (including, e.g., NPC), by administering to the subject the gene therapy composition of the present disclosure. Subjects at risk for the disease can be identified by, for example, one or a combination of diagnostic or prognostic assays known in the art (e.g., genetic assessment of the subject and/or phenotypic assessment). Administration of a prophylactic agent can occur prior to the detection of, e.g., NPC in a subject, or the manifestation of at least one symptom characteristic of the disease or disorder, such that the disease or disorder is prevented or, alternatively, delayed in its progression.

Another aspect of the disclosure pertains to methods of treating subjects therapeutically, i.e., altering the onset of symptoms of the disease or disorder. These methods can be performed in vitro (e.g., by culturing the cell with the gene therapy composition) or, alternatively, in vivo (e.g., by administering the gene therapy composition to a subject).

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the disclosure provides methods for tailoring an individual's prophylactic or therapeutic treatment with the gene therapy transgene of the present disclosure to that individual's drug response genotype. Pharmacogenomics allows a clinician or physician to target prophylactic or therapeutic treatments to patients who will most benefit from the treatment and to avoid treatment of patients who will experience toxic drug-related side effects.

### Gene Therapy Compositions

The disclosure, in part, pertains to a gene therapy composition comprising the NPC1-providing vectors as described herein. The gene therapy composition of the present disclosure can gain entry into a cell or tissue, e.g., a CNS cell or tissue, for ameliorating, treating and/or preventing at least one symptom of NPC disease or mitigating the complications, such as liver disease, neurological decline or seizures.

Advantageously, the gene therapy composition of the disclosure provides for a controlled delivery of an active gene, especially a therapeutic gene, to a site of action at an optimum rate and therapeutic dose. Thus, improvements in therapeutic index may be obtained by modulating the distribution of the active ingredient in the body and/or by modulating the promoter used in such gene therapy construct. Association of the gene therapy vector and/or viral vector containing such gene therapy vector with a delivery system enables, in particular, its specific delivery to the site of action or its controlled expression of a gene after targeting the action site. By reducing the amount of active gene therapy vector distributes to any compartments in which its presence is not desired, it is possible to increase the efficacy of the gene therapy agent, and to reduce any toxic side effects or even modify or restore activity of gene therapy agents. In this application, the capsid serotype can influence route of delivery, cellular transduction efficacy, and dose required for a therapeutic effect. The promoter of the vector further dictates cell type expression i.e., in all cells or only neurons and the degree to which expression occurs at the cellular level. As such, some promoters are stronger than others, and produce higher transgene expression. In another embodiment, microRNA (miRNA) binding sites are embedded in the 3′ untranslated region of the therapeutic transgene to provide a cell specific inhibition of translation if the NPC1 transgene product is toxic in one cell type compared to another. This approach would minimize off target expression in cell types other than neurons if needed.

The disclosure also relates to pharmaceutical or diagnostic compositions comprising the NPC1-including vectors or constructs of the disclosure and a pharmaceutically acceptable carrier. As such, direct RNA or DNA or modified forms if such, including peptide or covalently modified nucleic acids, injections in the brain or other locations are considered using the therapeutic transgenes described in this application. In another embodiment, nanoparticles containing codon optimized nucleic acids encoding NPC1 are used for gene delivery. In further embodiments, exosomes containing codon optimized nucleic acids (e.g., codon optimized for expression in human cells) encoding NPC1 are used for gene delivery. In other embodiments, exosomes containing codon optimized nucleic acids (e.g., codon optimized for expression in human cells) encoding NPC1 and NPC1 are used for gene delivery. The phrase “pharmaceutically acceptable carrier” is art recognized and includes a pharmaceutically acceptable material, composition or vehicle, suitable for administering compounds used in the methods described herein to subjects, e.g., mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in Remington's Pharmaceutical Sciences, Mack Publishing Company, a standard reference text in this field.

In certain embodiments, the present disclosure provides for a viral vector composition comprising a gene therapy agent (e.g., codon optimized NPC1 operably linked to a tissue-specific or systemic promoter, optionally operably linked to a PTD and/or a tag and within a plasmid corresponding to the form of viral delivery system employed, e.g., AAV viral vector plasmid) of the present disclosure. The active viral vector can be suitably formulated and introduced into the environment of the cell by any means that allows for a sufficient portion of the sample to enter the cell to induce expression of the gene therapy agent, if it is to occur. Many formulations for AAV and other vector-based gene therapy delivery are known in the art and can be used.

Such compositions can include the gene therapy agent and a pharmaceutically acceptable carrier. Supplementary active compounds can also be incorporated into the compositions. The AAV capsid can likewise be modified to improve uptake and viral stability, and alter tropism.

A gene therapy composition can be formulated to be compatible with its intended route of administration. Examples of routes of administration include parenteral, e.g., intravenous, intracranial, intrathecal, intraventricular, intramuscular, intrahepatic, intradermal, subcutaneous, oral (e.g., inhalation, buccal, sublingual, intranasal), transdermal (topical), transmucosal, and rectal administration. Nucleic acids can be delivered using electrical or magnetic stimulation, or direct physical uptake using hydrodynamic pressure. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Gene therapy compositions suitable for injectable use can include sterile aqueous solutions or dispersions and sterile powders for the extemporaneous preparation of sterile injectable solutions or dispersion. For intravenous administration, in certain embodiments, carriers can include physiological saline, bacteriostatic water, Cremophor EL® (BASF, Parsippany, N.J.) or phosphate buffered saline (PBS). However, the art as relates to a specific viral delivery vector will be known to the skilled artisan and will provide appropriate constituents for a gene therapy vector composition. A composition for injection must be sterile (apart from the AAV or other viral vector employed for delivery) and should be fluid to the extent that easy syringability exists. In certain embodiments, such compositions are stable under the conditions of manufacture and storage and are preserved against the contaminating action of microorganisms such as bacteria and fungi. Exemplary carriers can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin or albumin.

Sterile injectable solutions can be prepared by incorporating the gene therapy vectors disclosed herein in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

High titer AAV preparations can be produced using techniques known in the art, e.g., as described in U.S. Pat. No. 5,658,776 and Viral Vectors for Gene Therapy: Methods and Protocols, ed. Machida, Humana Press, 2003.

For administration by inhalation, gene delivery compositions can be delivered in the form of an aerosol spray from pressured container or dispenser which contains a suitable propellant, e.g., a gas such as carbon dioxide, or a nebulizer. Such methods include those described in U.S. Pat. No. 6,468,798, which is incorporated herein by reference.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For a compositions used in the method of the disclosure, the therapeutically effective dose can be estimated initially from cell culture assays. A dose may be formulated in animal models to achieve a circulating plasma concentration range that includes the IC₅₀ (i.e., the concentration of the test compositions which achieves a half-maximal inhibition of symptoms) as determined in cell culture. Such information can be used to more accurately determine useful doses in humans. Levels in plasma may be measured, for example, by high performance liquid chromatography.

Expression constructs of the disclosure can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057) or by any aforementioned delivery route. The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The expression constructs may be constructs suitable for use in the appropriate expression system and include, but are not limited to retroviral vectors, linear expression cassettes, modified mRNAs, plasmids and viral or virally-derived vectors, such as the AAV described herein, as known in the art. The nucleic acids can be modified covalently, such as peptide nucleic acids or base modified ribonucleic acids. Such expression constructs may include one or more promoters as detailed elsewhere herein.

Suitable amounts of a gene therapy composition must be introduced and these amounts can be empirically determined using standard methods.

The gene therapy composition can be formulated as a composition which comprises a pharmacologically effective amount of a transgene and/or viral vector containing a transgene, and pharmaceutically acceptable carrier. A pharmacologically or therapeutically effective amount refers to that amount of gene therapy agent effective to produce the intended pharmacological, therapeutic or preventive result. The phrases “pharmacologically effective amount” and “therapeutically effective amount” or simply “effective amount” refer to that amount of a gene therapy transgene effective to produce the intended pharmacological, therapeutic or preventive result. For example, if a given clinical treatment is considered effective when there is at least a 20% increase in a measurable parameter associated with a disease or disorder, a therapeutically effective amount of a gene therapy composition for the treatment of that disease or disorder is the amount necessary to effect at least a 20% increase in that parameter. In another example, if a given clinical treatment is considered effective when there is at least 10%, 20%, 30%, 40%, 50%, 60%, 70%, or 80% or more increase in a measurable parameter associated with a disease or disorder, a therapeutically effective amount of a gene therapy composition for the treatment of that disease or disorder is the amount necessary to effect at least a 10%, 20%, 30%, 40%, 50%, 60%, 70%, or 80% or more increase in that parameter.

### Markers of Transgene Expression/Activity

Toxicity and therapeutic efficacy of gene delivery compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Gene therapy compositions which exhibit high therapeutic indices are preferred. While gene therapy compositions that exhibit toxic side effects may be used, care should be taken to design a delivery system that targets such compositions to the site of affected tissue in order to minimize potential damage to uninfected cells and, thereby, reduce side effects.

In certain embodiments, membrane localization, including intracellular localization of a transgene or product thereof, e.g., NPC1, is assessed in the subject and/or in cells of the subject, e.g., using the FLAG®-tag. In other embodiments, assessment of the efficacy of NPC1 transgene delivery is performed via measurement of cholesterol uptake (e.g., endocytic cholesterol uptake) of the cells of a subject and/or via phenotypic assessment of a subject before and after administration of the AAV-NPC composition(s). Such assessment can be performed within days of administration of an AAV-NPC composition of the disclosure, or can be performed at a time of, e.g., one week, two weeks, three weeks, one month, two months, three months, four months, five months, six months, one year or more post-administration. The use of previously described biomarkers such as unesterified cholesterol, sphingomyelin, bis (monoacylglycero) phosphate, glucosylceramide, lactosylceramide, globotriaosylceramide, free sphingosine, gangliosides GM2 and GM3; galectin-3 (LGALS3) a pro-inflammatory molecule, and cathepsin D (CTSD), a lysosomal aspartic protease; and cholesterol oxidation products and neurosteroids such as cholestane-3β,5α,6β-triol (‘triol’), a cholesterol oxidation product that is elevated 10-fold in the plasma of NPC1 subjects, and 24(S)-hydroxycholesterol (24(S)-HC), an enzymatically generated oxygenated cholesterol that is reduced in the plasma of NPC1 subjects. Untargeted metabolomics are likewise envisioned to monitor the efficacy and activity of AAV gene therapy for NPC.

In certain embodiments, prior to treatment, a subject is assessed for the identity of genetic deficiency that has produced NPC in the subject—whether NPC1 or NPC2—and the subject is then administered is then administered an NPC1 transgene depending upon the outcome of such assessment. In another embodiment, the transgene encodes a transgene that has been codon optimized for human expression designated coNPC1. Methods for diagnosing NP disease can be found, for example, in U.S. Pat. Nos. 4,039,388, 5,686,240, 6,426,198, and 7,045,675 each of which are incorporated by reference.

The disclosure further provides a method to treat related disorders of unesterified cholesterol accumulation, such as atherosclerosis.

The level or activity of a transgene mRNA or polypeptide can be determined by a suitable method now known in the art or that is later developed, e.g., analyzing expression levels by PCR, hybridization, microarrays, or other similar methodologies. Suitable primers, probes, and oligonucleotides capable of performing such detection will be known and readily obtainable in the art. It can be appreciated that the method used to measure a transgenic mRNA and/or the expression of a transgenic protein can depend upon the nature of the transgene. Such measurements can be made on cells, cell extracts, tissues, tissue extracts or other suitable source material.

The determination of whether the expression of a transgene has been increased can be by a suitable method that can reliably detect changes in RNA or protein levels. In certain embodiments, the determination is made by introducing into the environment of a cell a gene therapy composition of the disclosure such that at least a portion of the gene therapy vector enters the cytoplasm (optionally, the nucleus; optionally, with nuclear chromosomal integration), and then measuring the level of the transgene RNA and/or polypeptide. The same measurement is made on identical untreated cells and the results obtained from each measurement are compared.

### Combination Therapies

It is contemplated that the compositions of the current disclosure can be combined with other proposed therapies (e.g., for NPC) to slow disease progression and ameliorate symptoms, even in patients with advanced disease. There are no published standards of care for NPC other than symptomatic treatment of disease manifestations—seizures are controlled as possible and supportive care is provided as needed. In one embodiment, AAV gene therapy would be combined with the pharmaceutical excipient 2-hydroxypropyl-β-cyclodextrin (HPBCD), such as VTS 270. In another, AAV gene therapy would be combined therapies shown to have modest efficacy in mouse models or cell culture studies including treatment with antioxidants such as N-acetylcysteine; vitamin E or derivatives such as α-tocepherol or δ-tocepherol; miglustat, a small imino sugar that partially inhibits glucosylceramide synthase and the synthesis of all glucosylceramide-based glycosphingolipids; curcumin to compensates for the lysosomal calcium defect by elevating cytosolic calcium; the non-steroidal anti-inflammatory drug ibuprofen or related compounds to reduce central nervous system inflammation; donepezil, a widely used acetylcholinesterase (AChE) inhibitor; or Histone deacetylase inhibitors (HDACi) such as vorinostat. In another embodiment, AAV gene therapy would be combined with other therapies that have a theoretical basis for efficiacy—such as those that influence cholesterol metabolism, but have limited efficacy to date. These include the cholesterol-lowering agents cholestyramine, lovastatin, and nicotinic acid as well as a low-cholesterol diet.

### Dosage

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

In certain embodiments, a suitable dosage unit of a transgene vector is in the range of 0.001 to 0.25 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. A gene therapy composition comprising the transgene can be administered once or on multiple occasions.

Data can be obtained from cell culture assays and animal studies to formulate a suitable dosage range for humans. The dosage of compositions of the disclosure can lie within a range of circulating concentrations that include the ED₅₀ (as determined by known methods) with little or no toxicity. The dosage may vary within this range depending upon the dosage form employed and the route of administration utilized. For a composition used in the method of the disclosure, the therapeutically effective dose can be estimated initially from cell culture assays. A dose may be formulated in animal models to achieve a circulating plasma concentration range of the composition that includes the IC₅₀ (i.e., the concentration of the test compound which achieves a half-maximal inhibition of symptoms) as determined in cell culture. Such information can be used to more accurately determine useful doses in humans. Levels of a gene therapy composition in plasma may be measured by standard methods, for example, by high performance liquid chromatography.

In certain embodiments, the dosage may be in terms of vector concentration. For example, the concentration of gene therapy vector described herein is at least: (a) 5, 6, 7, 8, 8.4, 9, 9.3, 10, 15, 20, 25, or 50×10¹² gc/ml; (b) 5, 6, 7, 8, 8.4, 9, 9.3, 10, 15, 20, 25, or 50×10⁹ tu/ml (“transducing units per ml”); (c) 5, 6, 7, 8, 8.4, 9, 9.3, 10, 15, 20, 25, or 50×10¹⁰ iu/ml (“international units per ml”), or (d) 5, 6, 7, 8, 8.4, 9, 9.3, 10, 15, 20, 25, or 50×10¹⁰ pfu/ml (“plaque forming units per ml”).

### Kits and/or Pharmaceutical Packages

The gene therapy compositions of the disclosure can be included in a kit and/or pharmaceutical package, container, pack, or dispenser together with instructions for administration.

The disclosure provides kits for the treatment or prevention of disease, e.g., NP disease, Type C. In one embodiment, the kit includes a therapeutic or prophylactic composition containing an effective amount of an agent of the disclosure (e.g., gene therapy vectors) in unit dosage form. In some embodiments, the kit comprises a sterile container which contains a therapeutic or prophylactic compound; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an agent of the disclosure is provided together with instructions for administering it to a subject having or at risk of developing a disease. The instructions will generally include information about the use of the composition for the treatment or prevention of the disease (e.g., NPC). In other embodiments, the instructions include at least one of the following: description of the compound; dosage schedule and administration for treatment or prevention of the disease or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### SUMMARY OF SEQUENCE LISTING

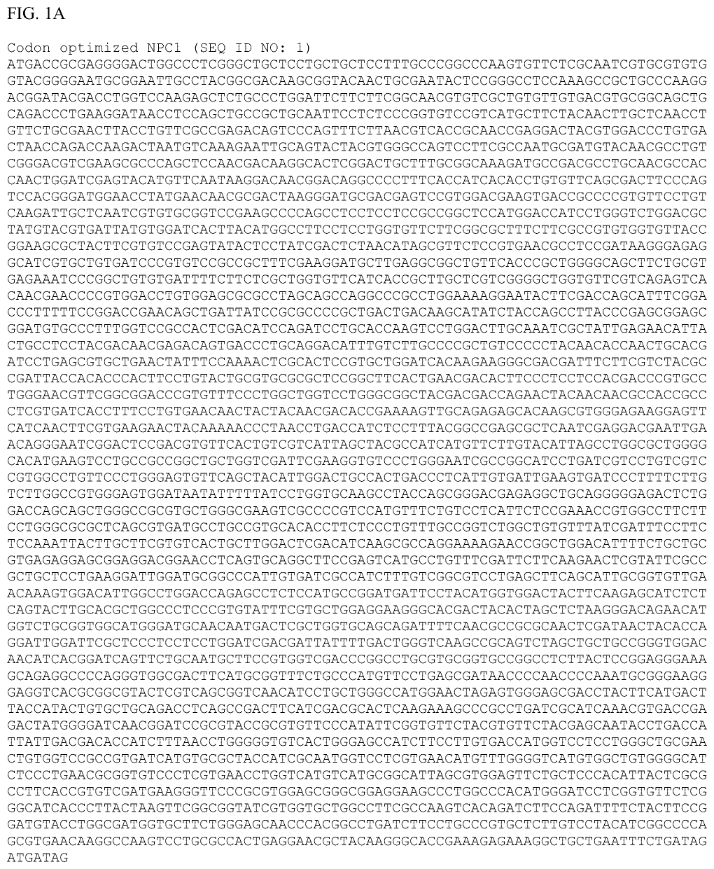

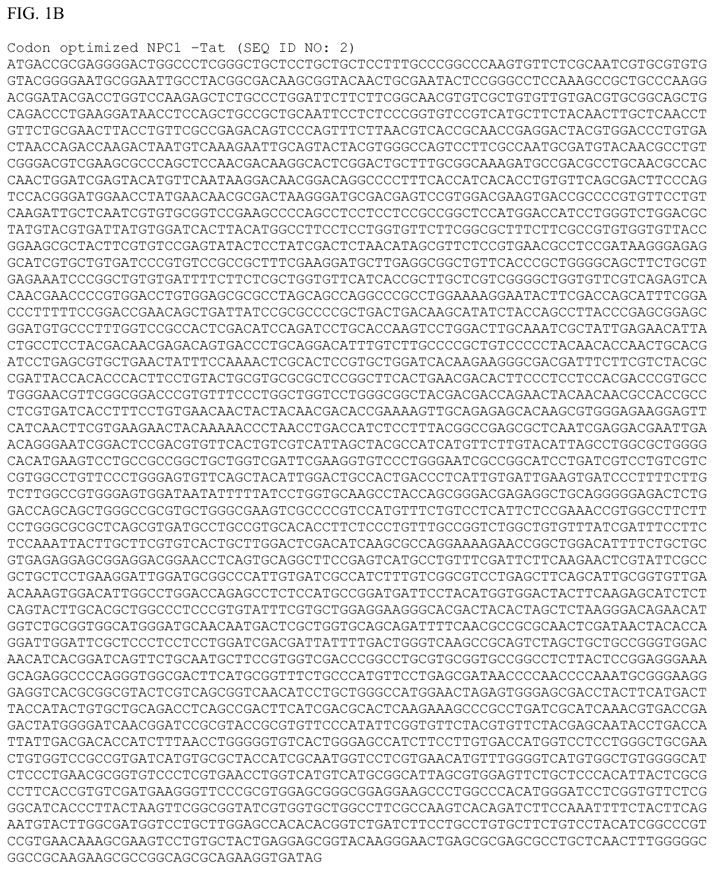

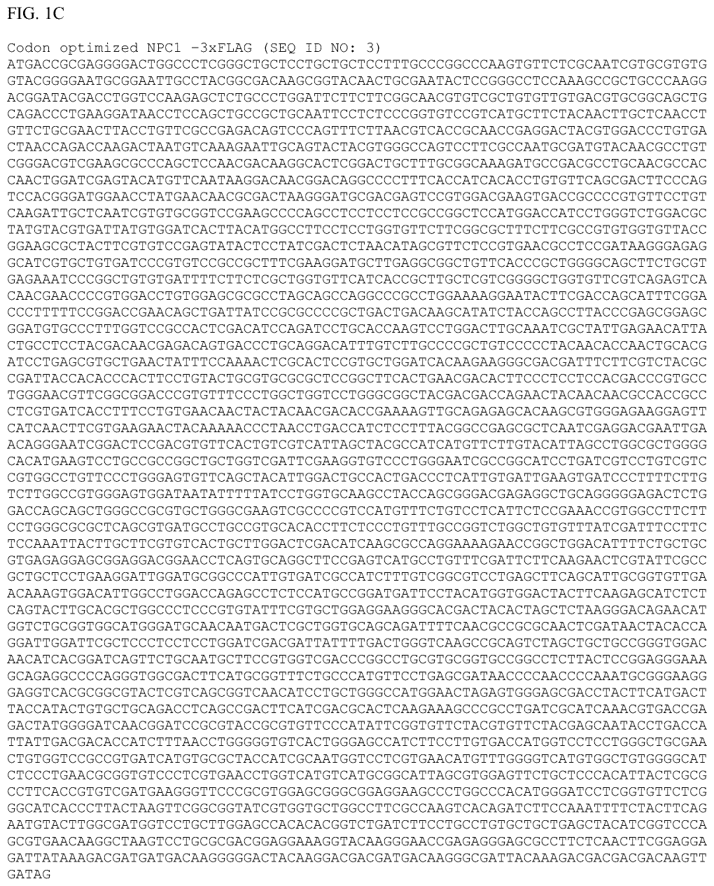

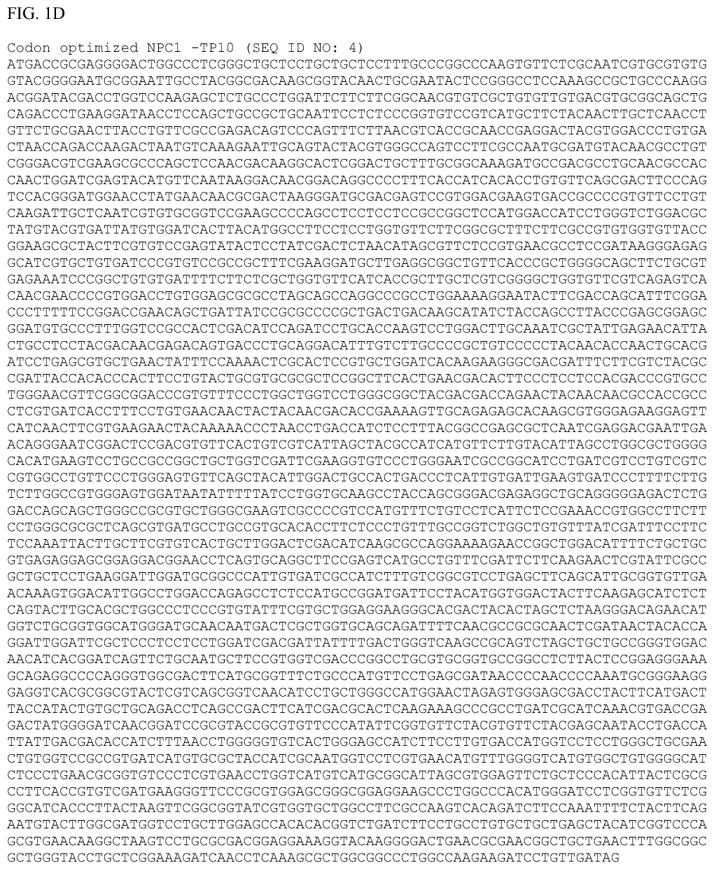

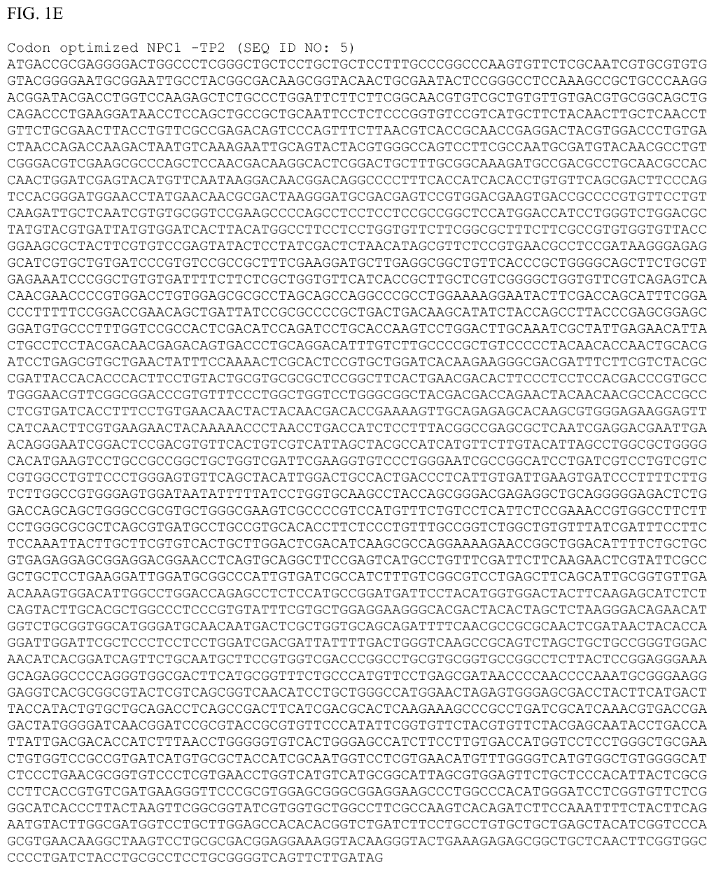

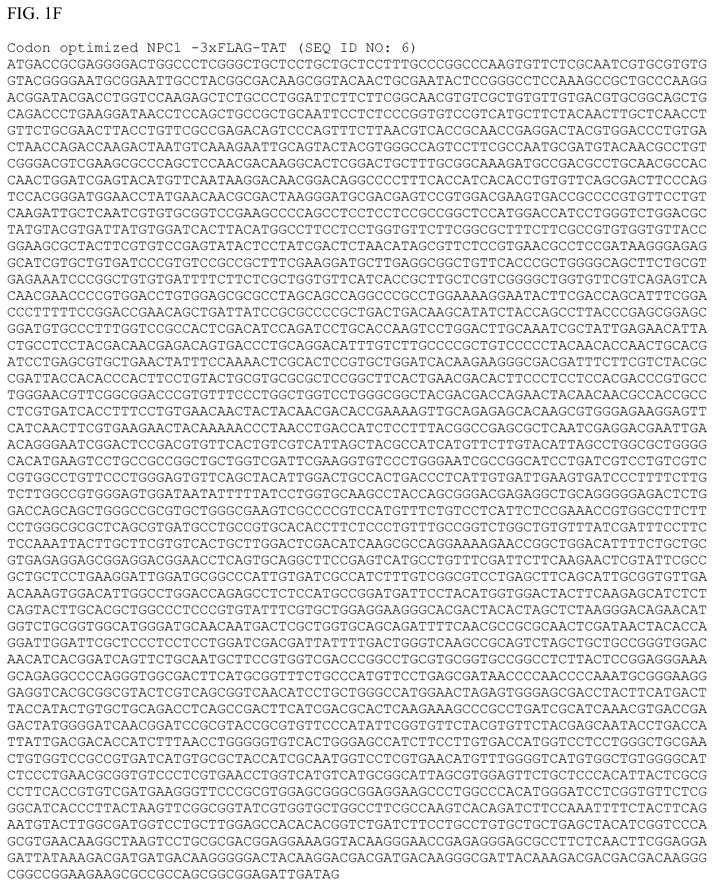

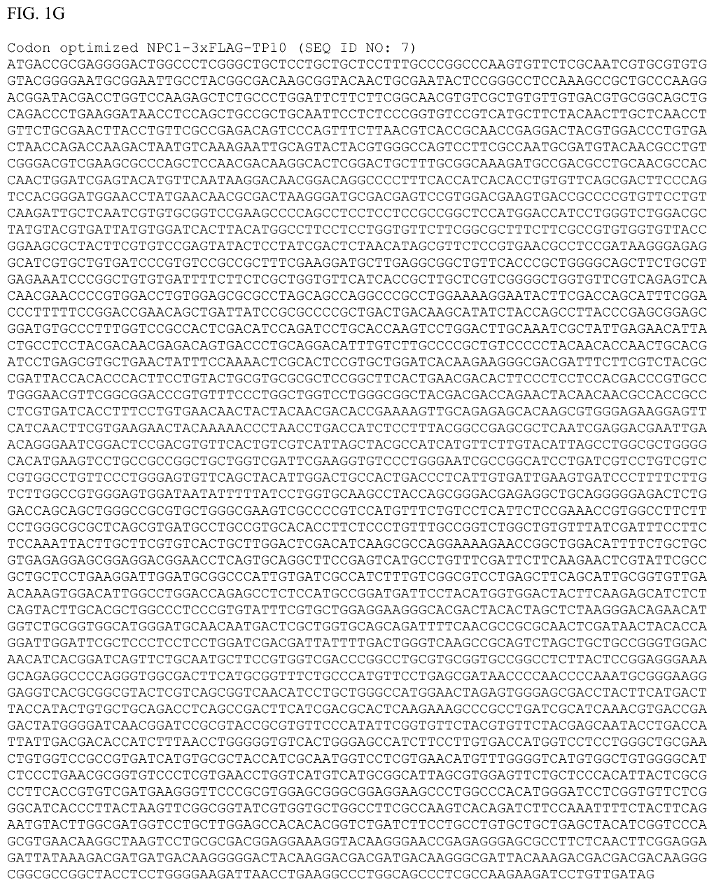

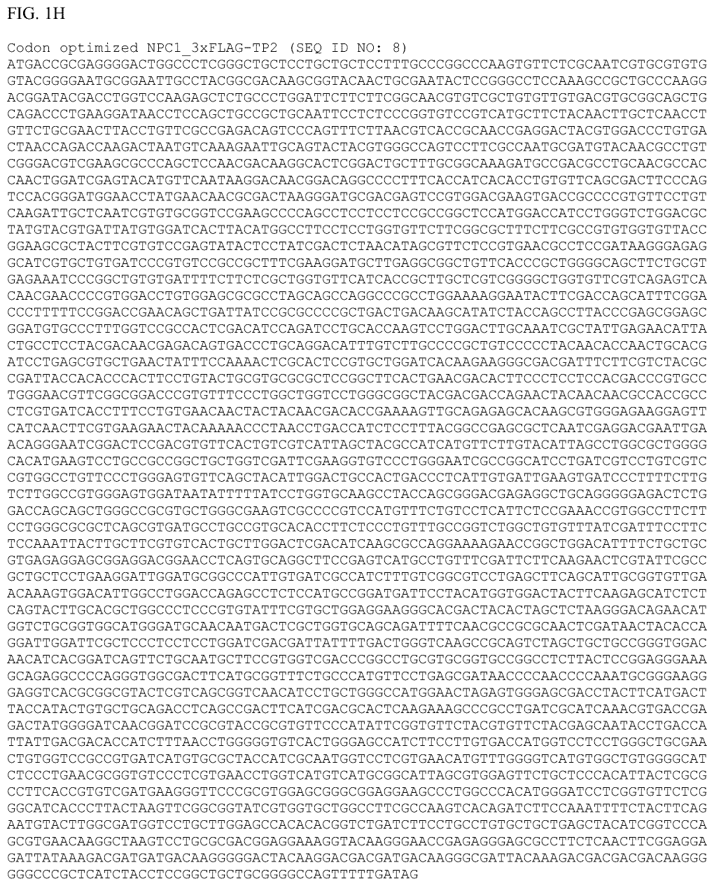

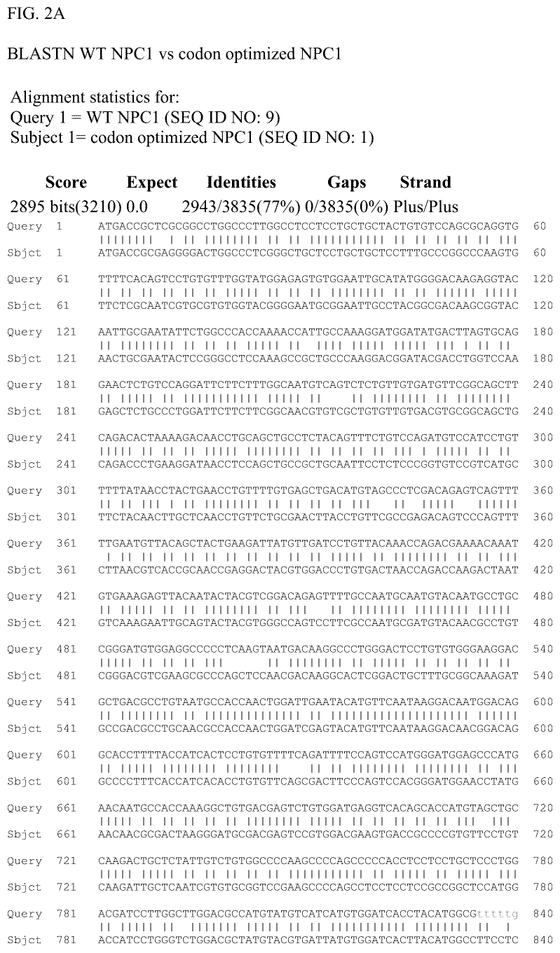

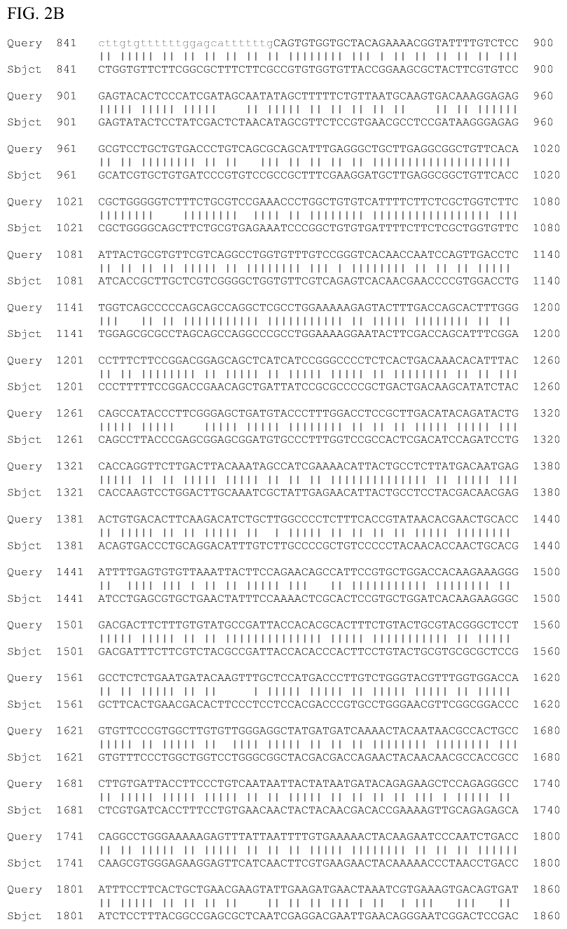

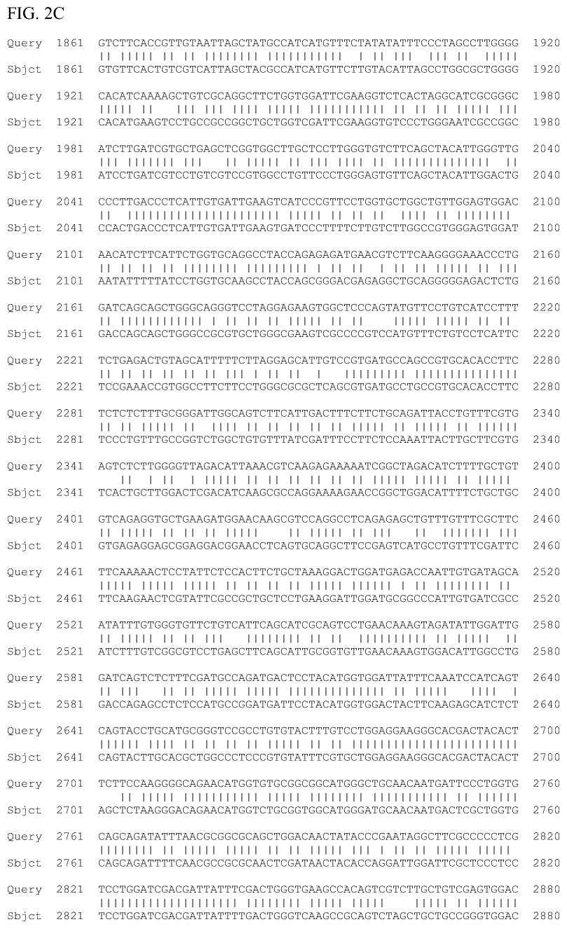

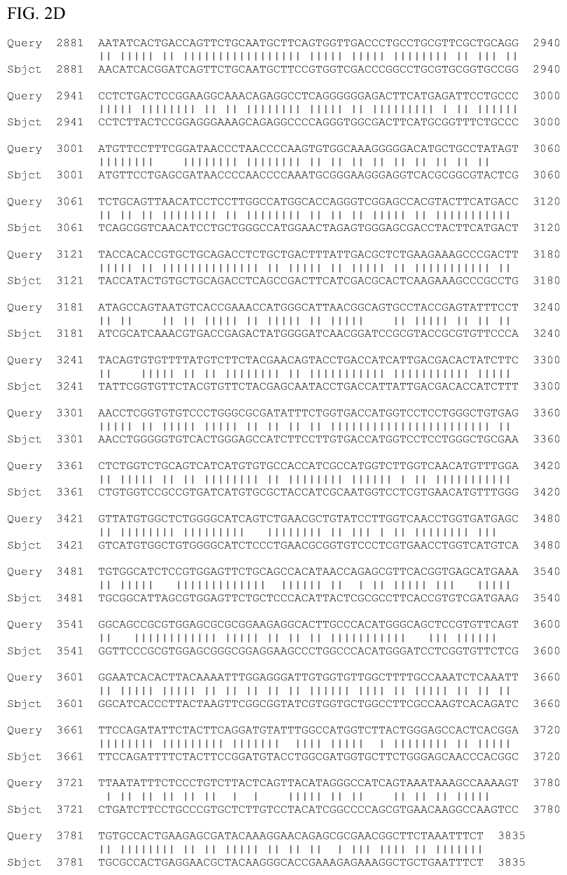

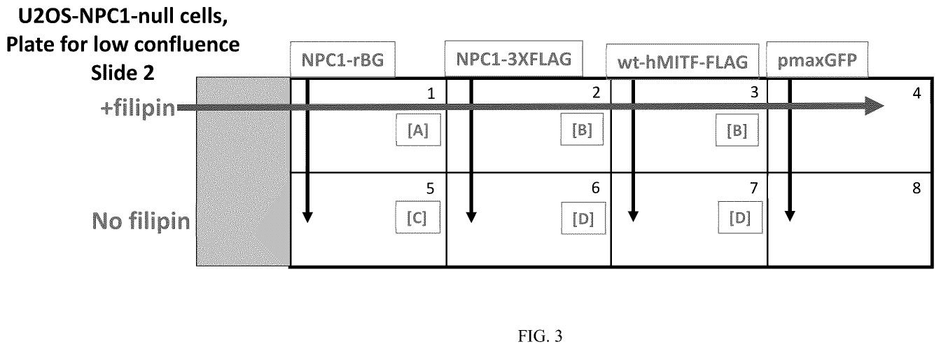

The specification includes a Sequence Listing appended herewith, which includes sequences, as follows:
SEQ ID NO: 1 (FIG. 1A—codon optimized NPC1);
SEQ ID NO: 2 (FIG. 1B—codon optimized NPC1-Tat);
SEQ ID NO: 3 (FIG. 1C—codon optimized NPC1-3xFLAG® tag);
SEQ ID NO: 4 (FIG. 1D—codon optimized NPC1-TP10);
SEQ ID NO: 5 (FIG. 1E—codon optimized NPC1-TP2);
SEQ ID NO: 6 (FIG. 1F—codon optimized NPC1-3xFLAG® tag-TAT);
SEQ ID NO: 7 (FIG. 1G—codon optimized NPC1-3xFLAG® tag-TP10);
SEQ ID NO: 8 (FIG. 1H—codon optimized NPC1-3xFLAG® tag-TP2);
SEQ ID NO: 9 (human NPC1 wild-type cDNA);
SEQ ID NO: 10 (truncated EF1α promoter (EF1αₜ));
SEQ ID NO: 11 (GenBank Accession No. BC117178; cDNA);
SEQ ID NO: 12 (GenBank Accession No. BC143756; cDNA);
SEQ ID NO: 13 (GenBank Accession No. AF258783.1; cDNA);
SEQ ID NO: 14 (GenBank Accession No. BC054539; cDNA);
SEQ ID NO: 15 (GenBank Accession No. BC151276; cDNA);
SEQ ID NO: 16 (GenBank Accession No. BC090541; cDNA);
SEQ ID NO: 17 (GenBank Accession No: NM_000271.4; cDNA);
SEQ ID NO: 18 (GenBank Accession No: NM_008720.2; cDNA);
SEQ ID NO: 19 (GenBank Accession No: NM_006432.3; cDNA);
SEQ ID NO: 20 (GenBank Accession No: NM_023409.4; cDNA);
SEQ ID NO: 21 (GenBank Accession No: NP_000262.2; protein);
SEQ ID NO: 22 (GenBank Accession No: NP_032746.2; protein);
SEQ ID NO: 23 (GenBank Accession No: NP_006423.1; protein);
SEQ ID NO: 24 (GenBank Accession No: NP_075898.1; protein);
SEQ ID NO: 25 (synthetic Intron S);
SEQ ID NO: 26 (Expression cassette for second generation vector miniEF1-coNPC1);
SEQ ID NO: 27 (Expression cassette for second generation vector miniEF1-coNPC1-TAT);
SEQ ID NO: 28 (Expression cassette for second generation vector miniEF1-coNPC1-3xFLAG tag);
SEQ ID NO: 29 (Expression cassette for second generation vector miniEF1-coNPC1-TP10);
SEQ ID NO: 30 (Expression cassette for second generation vector miniEF1-coNPC1-TP2)
SEQ ID NO: 31 (Expression cassette for second generation vector miniEF1-coNPC1-3xFLAG tag-TAT);
SEQ ID NO: 32 (Expression cassette for second generation vector miniEF1-coNPC1-3xFLAG tag-TP10);
SEQ ID NO: 33 (Expression cassette for seond generation vector miniEF1-coNPC1-3xFLAG tag-TP2);
SEQ ID NO: 34 (Expression cassette for third generation vector EF1s-coNPC1);
SEQ ID NO: 35 (Expression cassette for third generation vector EF1s-coNPC1-TAT);
SEQ ID NO: 36 (Expression cassette for third generation vector EF1s-coNPC1-3xFLAG tag);
SEQ ID NO: 37 (Expression cassette for third generation vector EF1s-coNPC1-3xFLAG tag-TAT);
SEQ ID NO: 38 (Expression cassette for fourth generation vector EF1s-intronS-coNPC1);
SEQ ID NO: 39 (Expression cassette for fourth generation vector EF1s-intronS-coNPC1-TAT);
SEQ ID NO: 40 (Expression cassette for fourth generation vector EF1s-intronS-coNPC1-3xFLAG tag);
SEQ ID NO: 41 (GenBank Accession No. BC063302; amino acid);
SEQ ID NO: 42 (GenBank Accession No. BC117178; amino acid);
SEQ ID NO: 43 (GenBank Accession No. BC143756; amino acid);
SEQ ID NO: 44 (GenBank Accession No. AF258783.1; amino acid);
SEQ ID NO: 45 (GenBank Accession No. BC054539; amino acid);
SEQ ID NO: 46 (GenBank Accession No. BC151276; amino acid);
SEQ ID NO: 47 (GenBank Accession No. BC090541; amino acid);
SEQ ID NO: 48 (Inverted Terminal Repeat 130 bases);
SEQ ID NO: 49 (Inverted Terminal Repeat 145 bases);
SEQ ID NO: 50 (deleted 59 base pair sequence);
SEQ ID NO: 51 (Mini EF1α promoter); and
SEQ ID NO: 52 (Short EF1α promoter (EF1α S)).

### EXAMPLES

The present disclosure is described by reference to the following Examples, which are offered by way of illustration and are not intended to limit the disclosure in any manner. Standard techniques well known in the art or the techniques specifically described below were utilized.

### Example 1

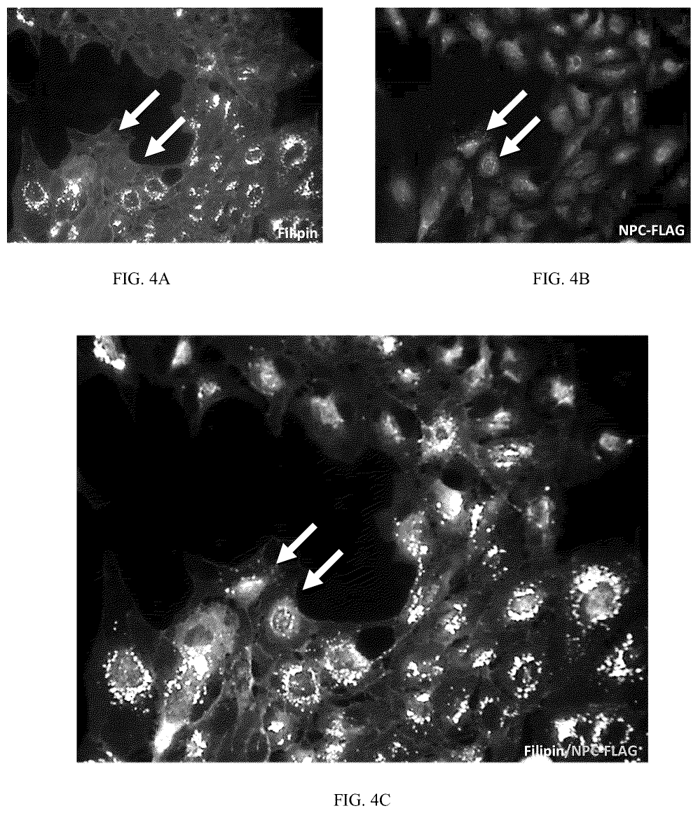

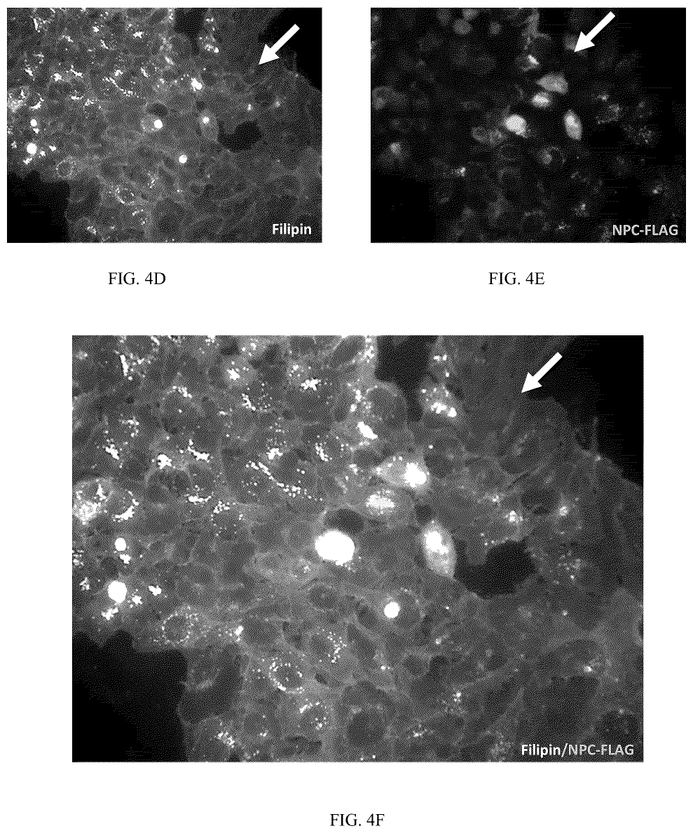

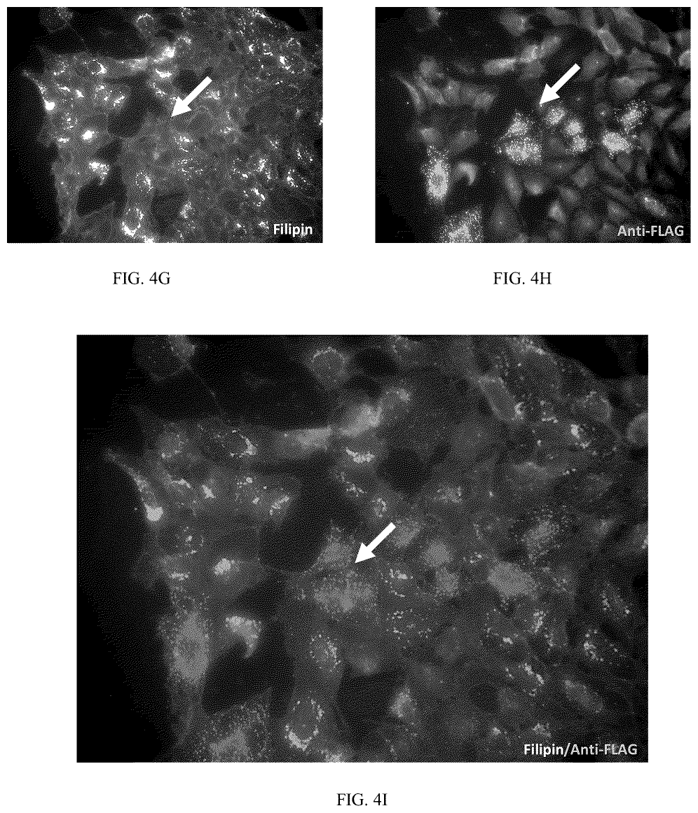

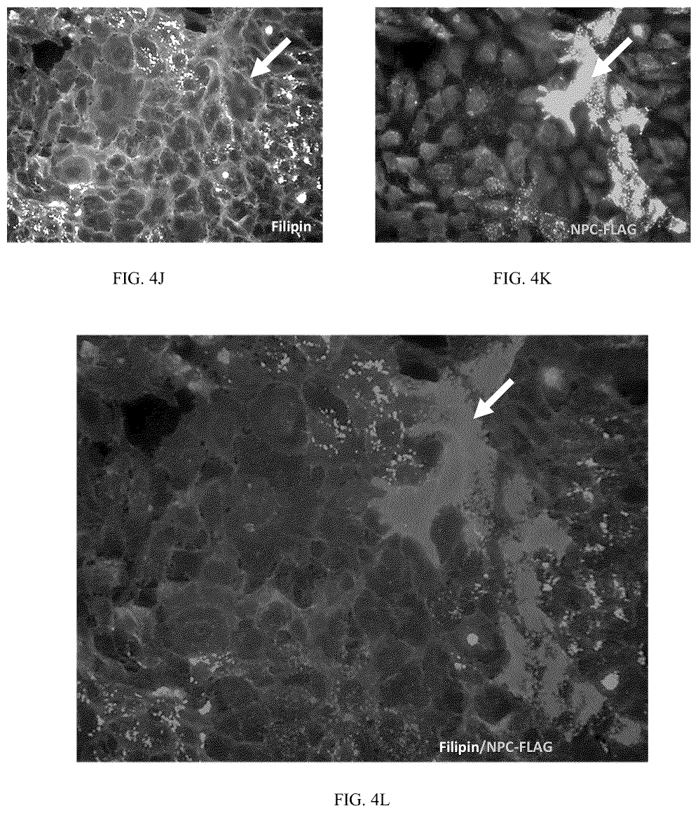

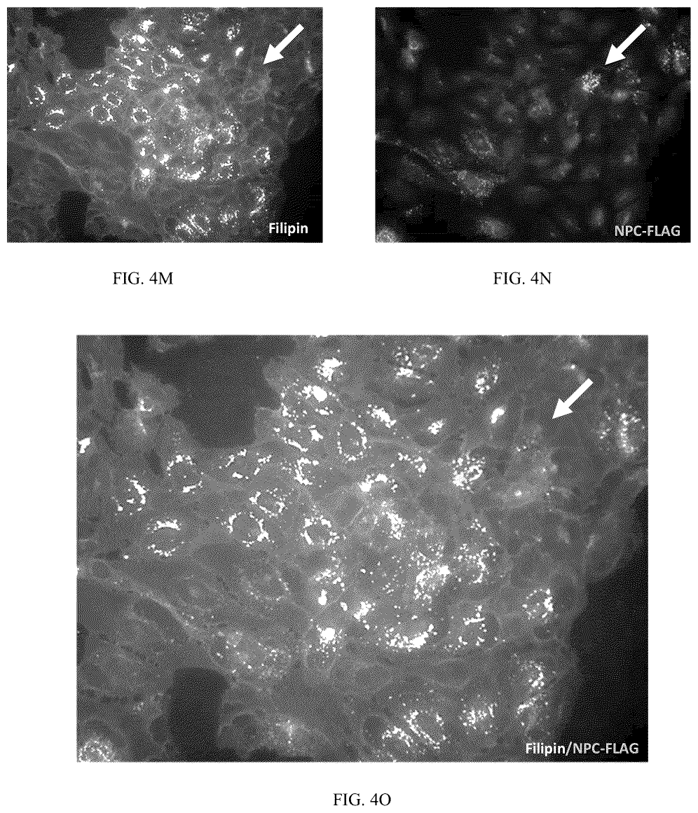

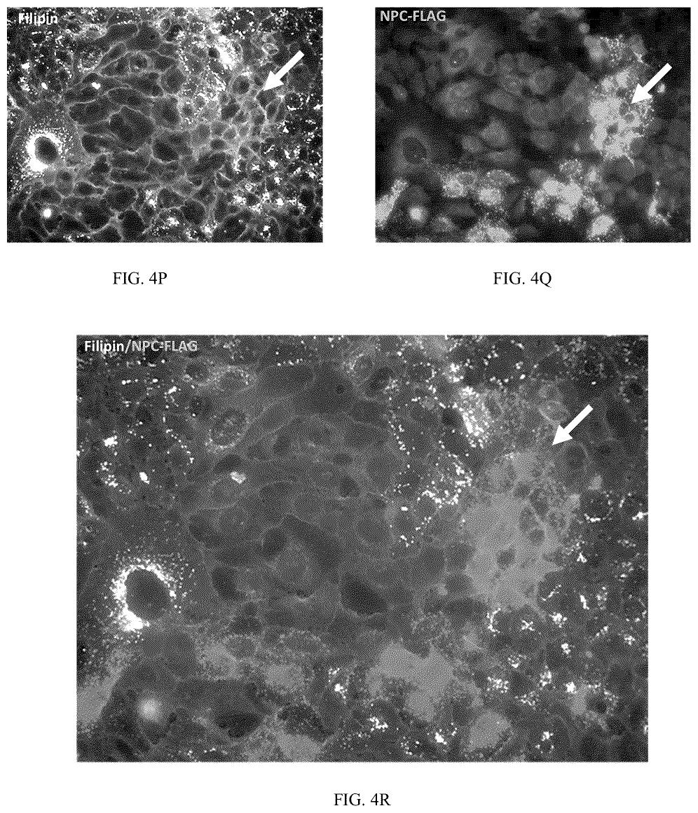

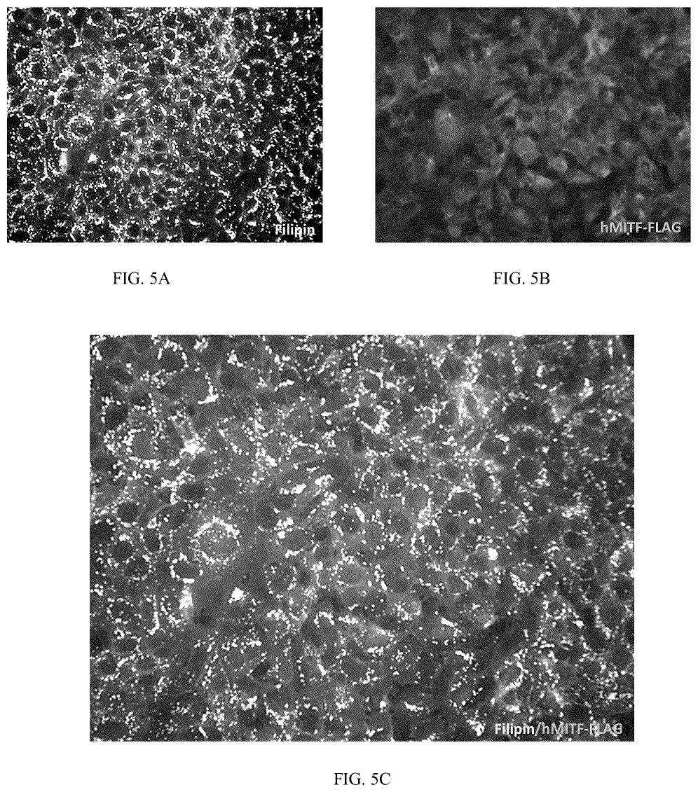

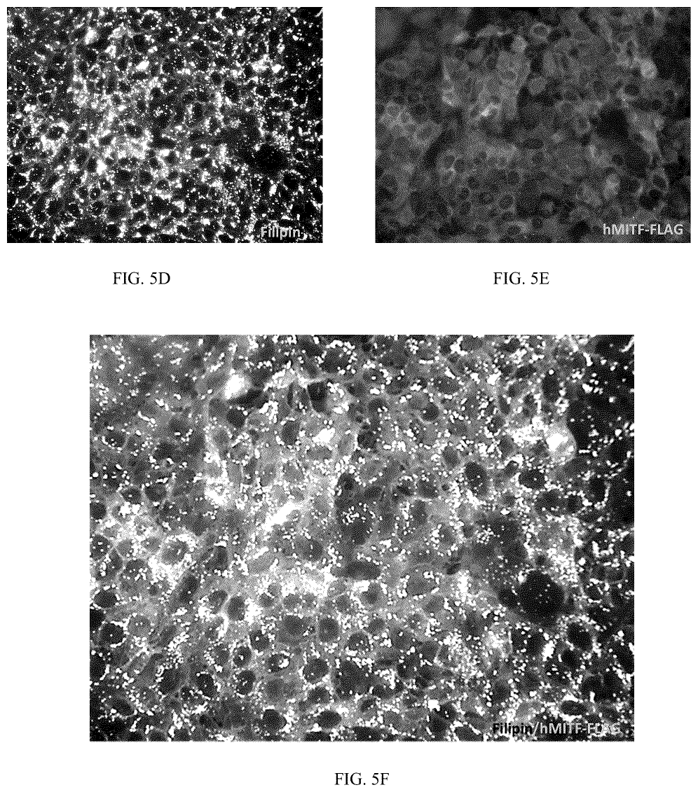

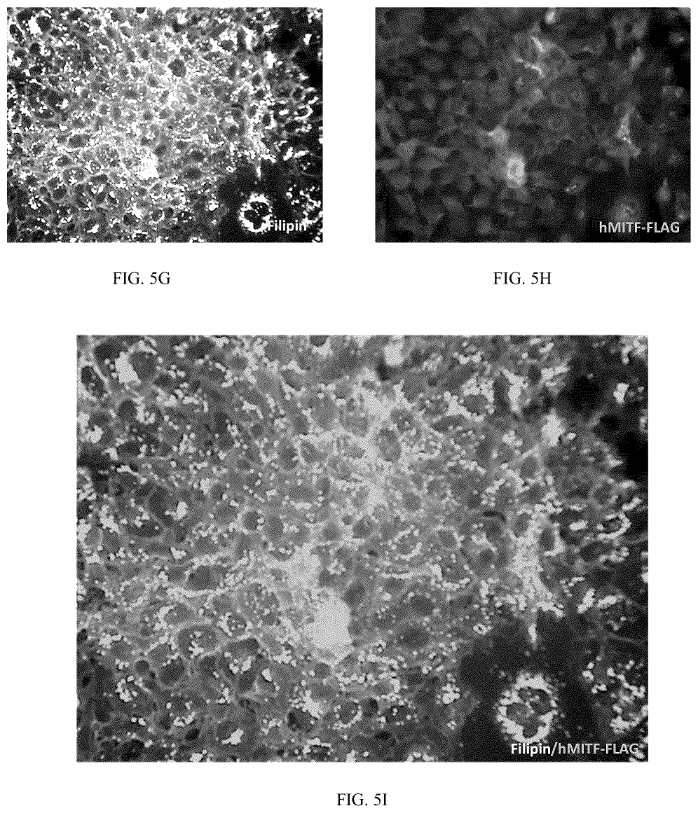

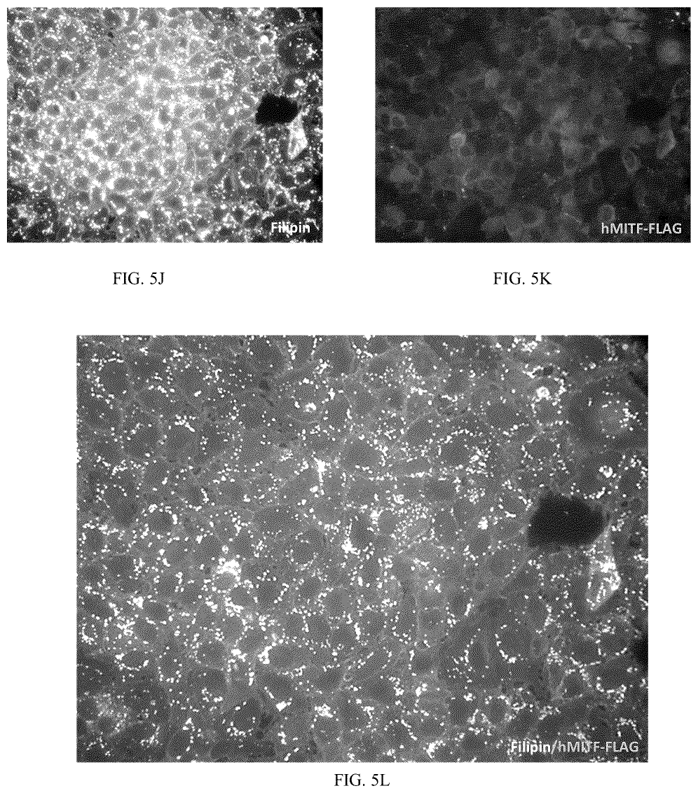

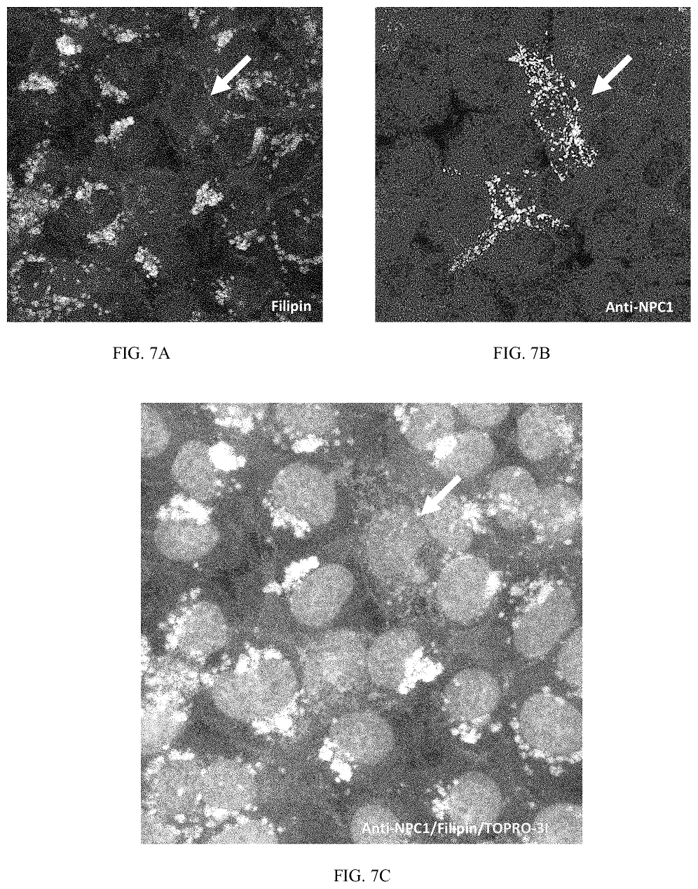

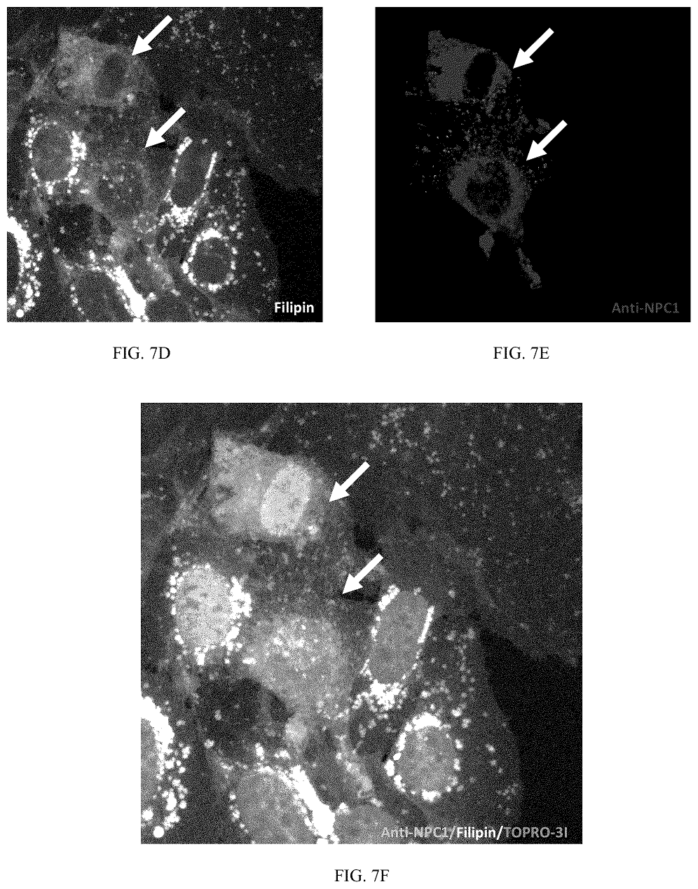

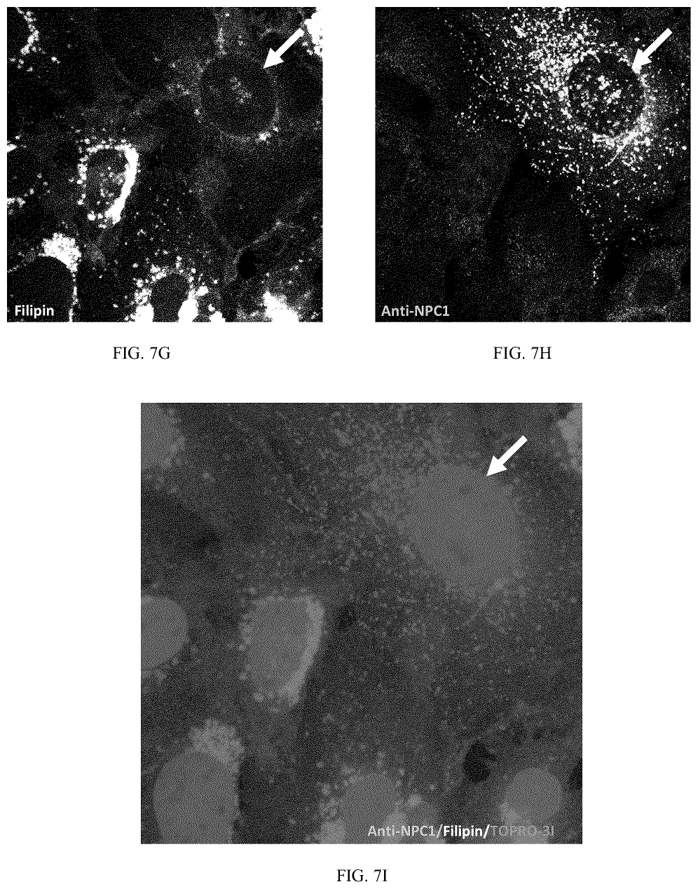

The constructs were synthesized and tested for NPC1 expression in a human mutant NPC1 cell line. The data can be seen in FIGS. 4A-4R (_{co}NPC1 expressing construct), FIGS. 5A-5R (hMITF-FLAG tag plasmid control construct), and FIGS. 7A-7I (intron containing _{co}NPC1 expressing construct). The cells were stained for filipin and either (1) hMITF-FLAG® tag (control), (2) _{co}NPC1-FLAG® tag, or (3) not stained with any FLAG® tag (FIGS. 7A-7C). Anti-NPC1 antibodies were added. The data demonstrates that coNPC1-FLAG® tag expressing cells cleared or significantly reduced the accumulated cellular cholesterol (a cellular hallmark of NPC), as shown by the decrease in filipin staining in cells expressing NPC1 (FIGS. 4A-4R and 7A-7I) as compared to those expressing the control hMITF-FLAG tag plasmid (FIGS. 5A-5R).

### Example 2

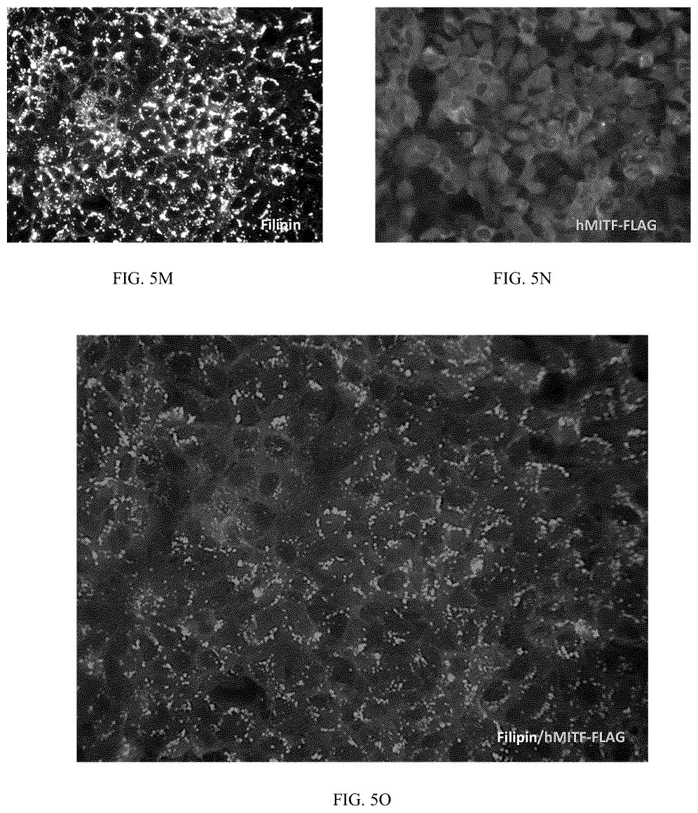

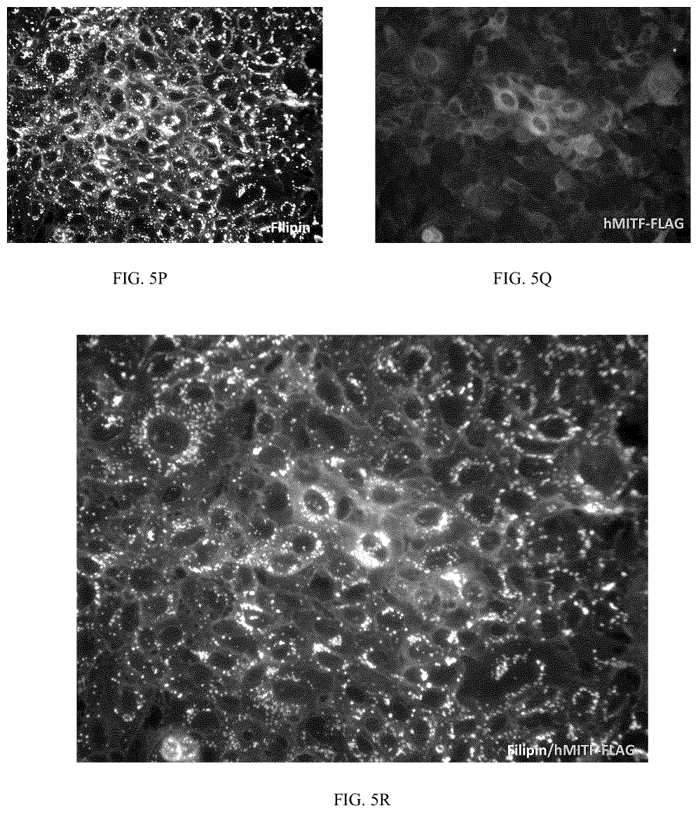

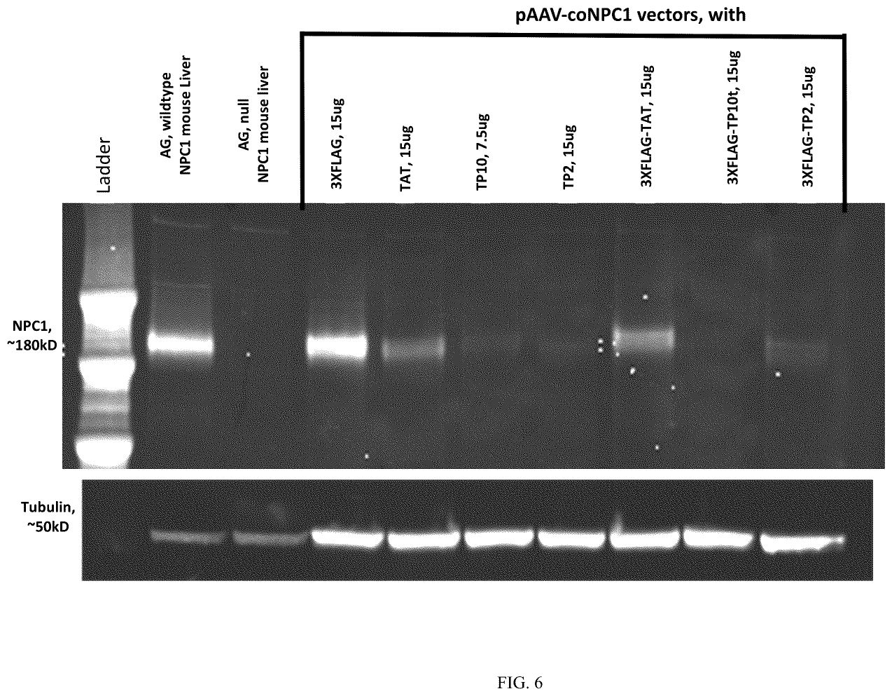

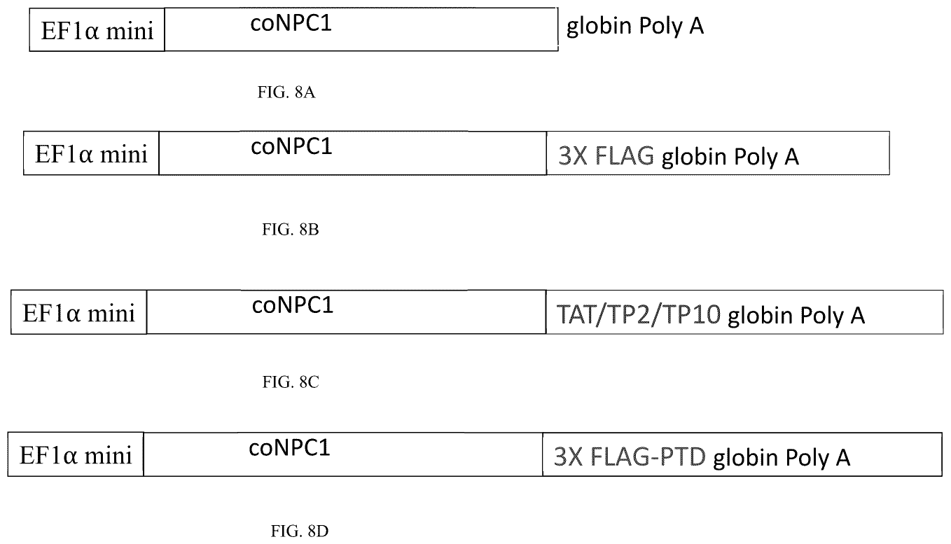

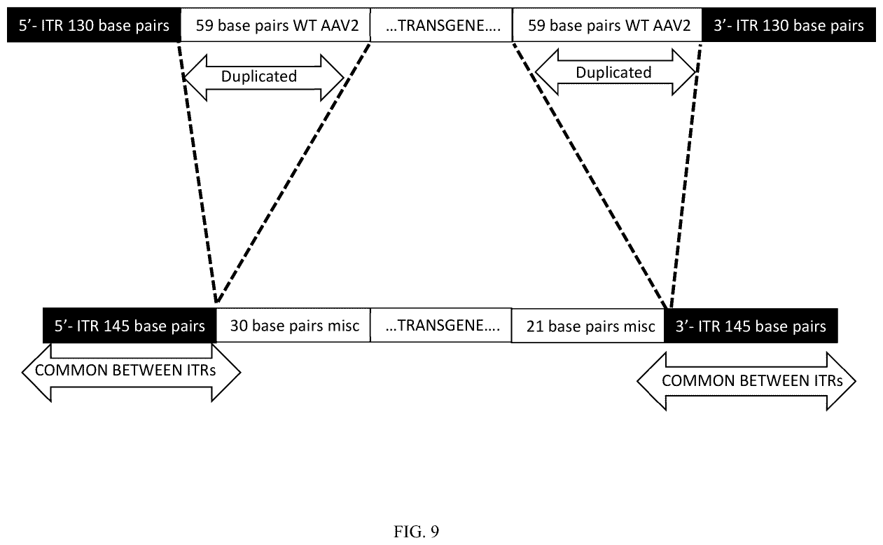

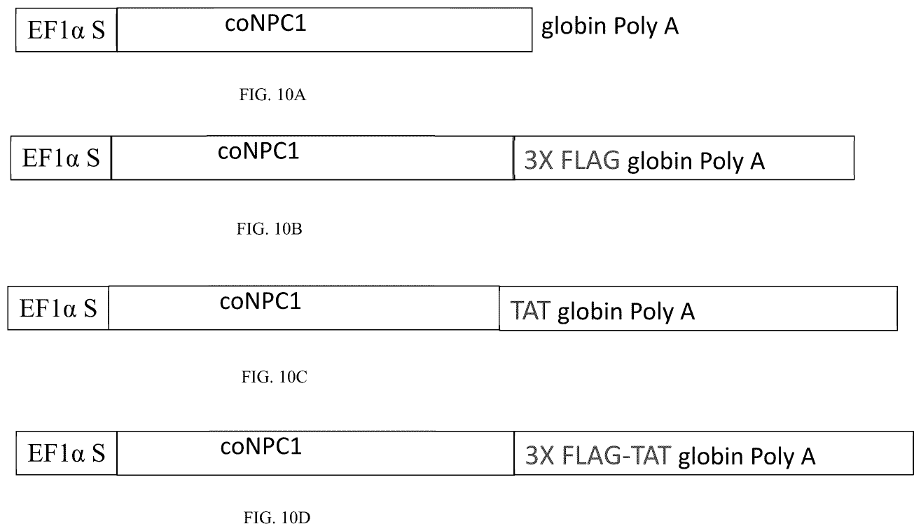

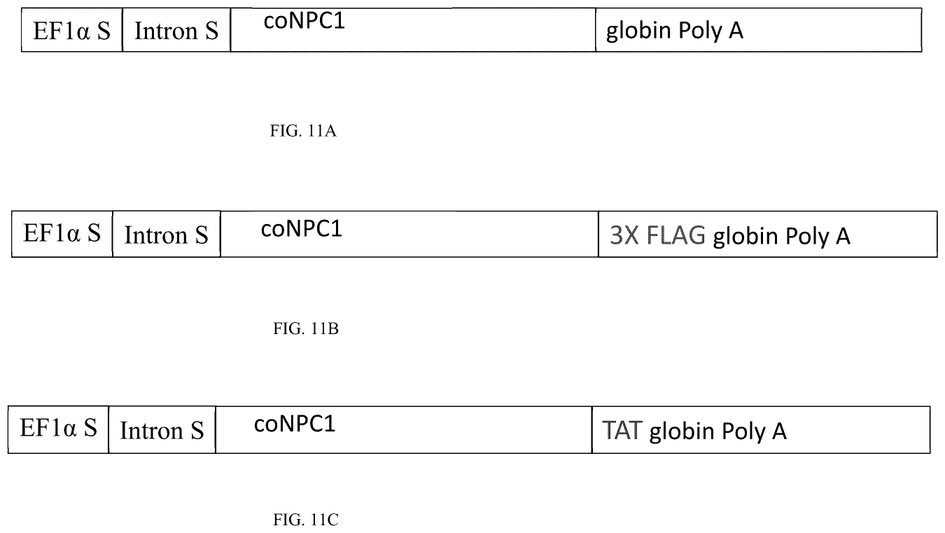

The expression cassettes of the following sequences were prepared as AAV plasmids: AAV mini EF1α-coNPC1 3xFLAG tag (FIG. 8B and SEQ ID NO: 28) AAV mini EF1α-coNPC1-tat (FIG. 8C and SEQ ID NO: 27)), AAV mini EF1α-coNPC1-TP10 (FIG. 8C and SEQ ID NO: 29), AAV mini EF1α-coNPC1-TP2 (FIG. 8C and SEQ ID NO: 30), AAV mini EF1α-coNPC1 3xFLAG tag-tat (FIG. 8D and SEQ ID NO: 31), AAV mini EF1α-coNPC1 3xFLAG tag-TP10 (FIG. 8D and SEQ ID NO: 32), AAV mini EF1α-coNPC1 3xFLAG tag-TP2 (FIG. 8D and SEQ ID NO: 33), AAV short EF1α-intronS-coNPC1 (FIG. 11A and SEQ ID NO: 38), AAV short EF1α-intronS-coNPC1 3xFLAG tag (FIG. 11B and SEQ ID NO: 40), and AAV short EF1α-intronS-coNPC1-tat (FIG. 11C and SEQ ID NO: 39). U20S-NPC1 knock out cells were transfected with 2 micrograms of each DNA construct using LIPOFECTAMINE (Invitrogen, Carlsbad, California, USA) to deliver the nucleic acid. After 48 hours, total cellular extracts were prepared from the transfected cells and subjected to Western analysis and compared to the expression of Npc1 in wild-type or Npc1 knock-out mouse liver extracts. As can be seen in FIG. 6, all AAV expression constructs tested, except for the AAV mini EF1α-coNPC1 3xFLAG tag-TP10 (FIG. 8C), produced robust expression in the NPC1 knock-out cell line after transfection, comparable to the expression of Npc1 in the wild-type mouse liver. The Western Blot analysis results for the AAV short EF1α-intronS-coNPC1 (FIG. 11A and SEQ ID NO: 38), AAV short EF1α-intronS-coNPC1 3xFLAG tag (FIG. 11B and SEQ ID NO: 40), and AAV short EF1α-intronS-coNPC1-tat (FIG. 11C and sEQ ID NO: 39) constructs are not shown.

### Discussion

The vectors (AAV) discussed above represent a new suite of therapeutic agents capable of treating human NPC1 deficiency and related conditions. Due to the genes being synthetic (i.e., codon-optimized), the significant nucleotide sequence divergence also will allow the NPC1 alleles to be easily and uniquely detected in preclinical models, and in patients.

The new codon optimized NPC1 alleles function in vivo as per the demonstration that the expression of NPC1-3xFLAG tag clears filipin from NPC1 null human cells, and localizes to the cytosol in a punctate pattern. Furthermore, the addition of a PTD such as tat, TP2, or TP10, or the combination of PTD and a tag is expressed from the AAV backbone as seen in Example 2.

The vectors of the present disclosure can be used to treat NPC1 deficiency by systemic gene delivery and/or by targeting delivery to an affected organ or tissue, such as to the CSF or deep brain structures, which can be affected in patients with NPC1.

The vectors of the present disclosure may also be used in a generalized fashion for other neurometabolic disorders if the tagged PTD elements are functional, and define a new approach to allow cross-correction of other disorder that have previously been constrained by cellular autonomy.

The vectors of the present disclosure may also be combined with novel AAV serotypes, such as PHP.B or Anc80, in order to allow potent CNS correction after peripheral gene delivery.

### SPECIFIC EMBODIMENTS

According to an aspect, the present disclosure provides a nucleic acid construct for the expression of a therapeutic amount of NPC1 in a cell, the construct comprising: a human codon-optimized NPC1 gene selected from the group consisting of SEQ ID NOs: 1-8.

In any aspect or embodiments described herein, the NPC1 is translationally fused to a protein transduction domain (PTD) to form a NPC1-PTD fusion protein, wherein the fusion protein is capable of cross-correcting non-transformed neighboring cells.

In any aspect or embodiments described herein, the PTD is HIV-Tat, transportin 10 (TP10), or TP2.

In any aspect or embodiments described herein, the NPC1 gene is under the control of a promoter.

In any aspect or embodiments described herein, the promoter is an EF1ₜ promoter.

In any aspect or embodiments described herein, the NPC1 is translationally fused to a FLAG®-tag moiety.

In any aspect or embodiments described herein, at least one of the cell, the neighboring cells, or both are neuronal cells.

In any aspect or embodiments described herein, the nucleic acid construct comprises a nucleic acid sequence selected from the group consisting of SEQ ID NOS: 1-8.

According to a further aspect, the present disclosure provides an expression vector comprising the nucleic acid construct of the present disclosure, and which is capable of expressing the NPC1 protein, the NPC1-PTD fusion protein, the NPC1-FLAG® tag fusion protein, the NPC1-PTD-FLAG® tag fusion protein, or the NPC1-FLAG® tag-PTD fusion protein in the cell.

According to another aspect, the present disclosure provides a cell comprising the expression vector of the present disclosure.

According to yet another aspect, the present disclosure provides a method of ameliorating, treating, or preventing Niemann-Pick disease, type C1 in a subject, the method comprising administering a therapeutically effective amount of an expression vector to at least one cell, the expression vector comprising a nucleic acid molecule comprising a human codon-optimized NPC1 gene selected from the group consisting of SEQ ID NOs: 1-8 that encodes an NPC1, wherein the expression vector is effective at ameliorating, treating, or preventing at least one symptom of Niemann-Pick disease, type C1 in the subject.

In any aspect or embodiments described herein, the NPC1 is translationally fused to a PTD to form a NPC1-PTD fusion protein, wherein the NPC1-PTD fusion protein is capable of cross-correcting non-transformed neighboring cells.

In any aspect or embodiments described herein, the PTD protein transduction domain is HIV-Tat, TP10, or TP2.

In any aspect or embodiments described herein, the NPC1 gene is under the control of a promoter.

In any aspect or embodiments described herein, the promoter is an EF1ₜ promoter.

In any aspect or embodiments described herein, the NPC1 is translationally fused to a FLAG®-tag moiety to form a NPC1-FLAG® tag fusion protein, a NPC1-PTD-FLAG® tag fusion protein, or a NPC1-FLAG® tag-PTD fusion protein.

In any aspect or embodiments described herein, at least one of the cell, the neighboring cells, or both are neuronal cells.

In any aspect or embodiments described herein, the nucleic acid molecule comprises a nucleic acid sequence selected from the group consisting of SEQ ID NO: 1-8.

In any aspect or embodiments described herein, the NPC1 gene may be under the control of a promoter, wherein the promoter is may be a truncated EF1α promoter (EF1αₜ promoter) or a mini EF1α promoter (EF1α promoter) or the short EF1α promoter (EF1α S).

In any aspect or embodiments described herein, the nucleic acid construct comprises an expression cassette comprising a nucleotide sequence selected from the group consisting of SEQ ID NOs: 26-38.

In any aspect or embodiments described herein, the gene therapy vector may comprise first and second full-length, wild type AAV inverted terminal repeats (ITRs) that flank the transgene. In any aspect or embodiments described herein, besides the ITRs, the nucleic acid construct may be devoid of any other sequence from the wild-type AAV genome.

In any aspect or embodiments described herein, the first and second AAV ITRs are AAV2 ITRs. In a particular embodiment, the first and second AAV ITRs each comprise the nucleotide sequence of SEQ ID NO: 48. In another particular embodiment, the first and second AAV ITRs each comprise the nucleotide sequence of SEQ ID NO: 49.

In any aspect or embodiments described herein, the _{co}NPC1 protein, the _{co}NPC1-PTD fusion protein and NPC1-tag fusion protein can be included in a vector, more particularly, in an expression cassette, with a synthetic intron sequence (SEQ ID NO: 25) which may enhance expression by providing more effective splicing of the transgene as compared to the vector which does not comprise the synthetic intron sequence.

In any aspect or embodiments described herein, the NPC1 gene is under the control of a promoter, and the Intron S is positioned between the promoter and the NPC1 gene. In a particular embodiment, the Intron S comprises the nucleotide sequence of SEQ ID NO: 25.

In any aspect or embodiments described herein, the nucleic acid construct may further comprise a nucleotide sequence encoding an antibiotic resistance marker. In an embodiment, the antibiotic resistance marker is an ampicillin resistance marker. In any aspect or embodiments described herein, the antibiotic resistance marker is a kanamycin resistance marker.

In any aspect or embodiments described herein, the nucleic acid construct comprises an expression vector which is capable of expressing the NPC1 protein, the NPC1-PTD fusion protein, or the NPC1 fusion protein, optionally comprising a tag moiety.

In any aspect or embodiments described herein, the invention comprises a cell comprising the expression vector, wherein the cell produces the expression vector and/or the cell expresses the NPC1 protein.

In any aspect or embodiments described herein, the invention comprises a method of ameliorating, treating, or preventing Niemann-Pick disease, type C1 in a subject, the method comprising administering a therapeutically effective amount of an expression vector to at least one cell, the expression vector comprising a nucleic acid molecule comprising a human codon-optimized NPC1 gene, wherein the expression vector is effective at of ameliorating, treating, or preventing at least one symptom of Niemann-Pick disease, type C1 in the subject.

In any aspect or embodiments described herein, the NPC1 of the inventive method is translationally fused to a PTD to form a NPC1-PTD fusion protein, wherein the NPC1-PTD fusion protein is capable of cross-correcting non-transformed neighboring cells. In particular embodiments, the PTD protein transduction domain may be HIV-Tat, TP10, or TP2.

In any aspect or embodiments described herein, the NPC1 gene of the inventive method is under the control of a promoter. In any aspect or embodiments described herein, the promoter may be a truncated EF1α promoter (EF1αₜ promoter) or a mini EF1α promoter (EF1α promoter) or the short EF1α promoter (EF1α S).

In any aspect or embodiments described herein, the NPC1 gene of the inventive method may be translationally fused to a tag moiety to form a NPC1-tag fusion protein, a NPC1-PTD-tag fusion protein, or a NPC1-tag-PTD fusion protein.

In any aspect or embodiments described herein, the nucleic acid molecule of the inventive method comprises a nucleic acid sequence selected from the group consisting of SEQ ID NO: 1-8.

In any aspect or embodiments described herein, the expression vector of the inventive method may comprise an expression cassette, with a synthetic intron sequence (SEQ ID NO: 25), which sequence may enhance expression by providing more effective splicing of the transgene as compared to the gene therapy vector which does not comprise the synthetic intron sequence. In any aspect or embodiments described herein, the NPC1 gene of the inventive method is under the control of a promoter, and the Intron S is positioned between the promoter and the NPC1 gene. In a particular embodiment, the Intron S comprises the nucleotide sequence of SEQ ID NO: 25.

In any aspect or embodiments described herein, the inventive method comprises a nucleic acid construct comprising an expression cassette comprising a nucleotide sequence selected from the group consisting of SEQ ID NOs: 26-38.

In any aspect or embodiments described herein, the expression vector of the inventive method may comprise first and second full-length, wild type AAV inverted terminal repeats (ITRs) that flank the transgene. In any aspect or embodiments described herein, besides the ITRs, the nucleic acid construct may be devoid of any other sequence from the wild-type AAV genome. In an embodiment, the first and second AAV ITRs are AAV2 ITRs. In a particular embodiment, the first and second AAV ITRs each comprise the nucleotide sequence of SEQ ID NO: 48. In any aspect or embodiments described herein, the first and second AAV ITRs each comprise the nucleotide sequence of SEQ ID NO: 49.

In any aspect or embodiments described herein, inventive method may further comprise a nucleotide sequence encoding an antibiotic resistance marker. In an embodiment, the antibiotic resistance marker is an ampicillin resistance marker. In another embodiment, the antibiotic resistance marker is a kanamycin resistance marker.

In any aspect or embodiments described herein, the nucleic acid construct may comprise first and second AAV ITRs, the human codon-optimized NPC1 gene, and one or more of a nucleotide sequence encoding a PTD, a promoter, an Intron S, and a nucleotide sequence encoding a tag, wherein the NPC1 gene and all of the one or more of the nucleotide sequence encoding the PTD, the promoter, and the nucleotide sequence encoding the tag are positioned between the first and second AAV ITRs. In any aspect or embodiments described herein, the nucleic acid construct may comprise first and second AAV ITRs, the human codon-optimized NPC1 gene, and all of a nucleotide sequence encoding a PTD, a promoter, an Intron S, and a nucleotide sequence encoding a tag, wherein the NPC1 gene and all of the one or more of the nucleotide sequence encoding the PTD, the promoter, and the nucleotide sequence encoding the tag are positioned between the first and second AAV ITRs.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

Reference for Anc AAVs and AAV Anc80: Zinn E, Pacouret S, Khaychuk V, Turunen H T, Carvalho L S, Andres-Mateos E, Shah S, Shelke R, Maurer A C, Plovie E, Xiao R, Vandenberghe L H. In Silico Reconstruction of the Viral Evolutionary Lineage Yields a Potent Gene Therapy Vector. Cell Rep. 2015 Aug. 11;12(6):1056-68. doi: 10.1016/j.celrep.2015.07.019. Epub 2015 Jul. 30. PubMed PMID: 26235624; PubMed Central PMCID: PMC4536165

| | | ITR | | | Tag or | 5′ | | SEQ ID NO. of |
|---|---|---|---|---|---|---|---|---|
| Vector | Generation | (bases) | Antibiotic | Transgene | PTD | Intron | Studies | expression cassette |
| AAV-mini EF1α-coNPC1- | Second | 130 | ampicillin | codon | none | not | | mini EF1α-coNPC1 |
| AMP | | | | optimized | | present | | (SEQ ID NO: 26) |
| | | | | NPC1 | | | | |
| AAV-mini EF1α-coNPC1- | Second | 130 | ampicillin | codon | 3xFLAG | not | Transfection | mini EF1α-coNPC1- |
| 3XFLAG tag AMP | | | | optimized | | present | | 3XFLAG tag |
| | | | | NPC1 | | | | (SEQ ID NO: 28) |
| AAV-mini EF1α-coNPC1- | Second | 130 | ampicillin | codon | TAT | not | Transfection, | mini EF1α-coNPC1- |
| TAT AMP | | | | optimized | | present | IHC, Filipin | TAT (SEQ ID NO: 27) |
| | | | | NPC1 | | | | |
| AAV-mini EF1α-coNPC1- | Second | 130 | ampicillin | codon | TP2 | not | Transfection | mini EF1α-coNPC1-TP2 |
| TP2 AMP | | | | optimized | | present | | (SEQ ID NO: 30) |
| | | | | NPC1 | | | | |
| AAV-mini EF1α-coNPC1- | Second | 130 | ampicillin | codon | TP10 | not | Transfection | mini EF1α-coNPC1-TP10 |
| TP10 AMP | | | | optimized | | present | | (SEQ ID NO: 29) |
| | | | | NPC1 | | | | |
| AAV-mini EF1α-coNPC1- | Second | 130 | ampicillin | codon | 3XFLAG- | not | Transfection | mini EF1α-CONPC1- |
| 3XFLAG tag-TAF AMP | | | | optimized | TAT | present | | 3XFLAG tag-TAT |
| | | | | NPC1 | | | | (SEQ ID NO: 31) |
| AAV-mini EF1α-coNPC1- | Second | 130 | ampicillin | codon | 3XFLAG- | not | Transfection | mini EF1α-coNPC1- |
| 3XFLAG tag-TP2 AMP | | | | optimized | TP2 | present | | 3XFLAG tag-TP2 |
| | | | | NPC1 | | | | (SEQ ID NO: 33) |
| AAV-mini EF1α-coNPC1- | Second | 130 | ampicillin | codon | 3XFLAG- | not | Transfection | mini EF1α-coNPC1- |
| 3XFLAG tag-TP10 AMP | | | | optimized | TP10 | present | | 3XFLAG tag-TP10 |
| | | | | NPC1 | | | | (SEQ ID NO: 32) |
| AAV- EF1α S-coNPC1-AMP | Third | 145 | ampicillin | codon | none | not | | EF1α S-coNPC1 |
| | | | | optimized | | present | | (SEQ ID NO: 34) |
| | | | | NPC1 | | | | |
| AAV-EF1α S_NPC1_3xFLAG | Third | 145 | ampicillin | codon | 3xFLAG | not | grown as an | EF1α S_NPC1_3XFLAG |
| tag AMP | | | | optimized | | present | AAV9 vector | tag (SEQ ID NO: 36) |
| | | | | NPC1 | | | | |
| AAV-EF1α S-NPC1_TAT | Third | 145 | ampicillin | codon | TAT | not | | EF1α S-NPC1_TAT |
| AMP | | | | optimized | | present | | (SEQ ID NO: 35) |
| | | | | NPC1 | | | | |
| AAV-EF1α-coNPC1- | Third | 145 | ampilcillin | codon | 3XFLAG- | not | | EF1α S-coNPC1- |
| 3XFLAG tag-TAT AMP | | | | optimized | TAT | present | | 3XFLAG-TAT |
| | | | | NPC1 | | | | (SEQ ID NO: 37) |
| AAV-EF1α | Fourth | 145 | kanamycin | codon | none | present | Transfection, | EF1α S-intronS-NPC1 |
| S-intronS-NPC1-KANA | | | | optimized | | | IHC, Filipin | (SEQ ID NO: 38) |
| | | | | NPC1 | | | | |
| AAV-EF1α | Fourth | 145 | kanamycin | codon | 3xFLAG | present | Transfection, | EF1α S-intronS- |
| S-intronS-BPC1_3XFLAG | | | | optimized | | | IHC, Filipin | NPC1_3XFLAG tag |
| tag-KANA | | | | NPC1 | | | | (SEQ ID NO: 40) |
| AAV-EF1α | Fourth | 145 | kanamycin | codon | TAT | present | Transfection, | EF1α S-intronS- |
| S-intronS-NPC1_TAT-KANA | | | | optimized | | | IHC, Filipin | NPC1_TAT |
| | | | | NPC1 | | | | (SEQ ID NO: 39) |

1. A method of ameliorating, treating, or preventing Niemann-Pick disease, type C1 in a subject, the method comprising administering to the subject a nucleic acid construct encoding a Niemann-Pick disease, type C1 (NPC1) fusion protein, the construct comprising a human codon-optimized NPC1 gene, and wherein the NPC1 is translationally fused to a protein transduction domain (PTD) to form a NPC1-PTD fusion protein, and the PTD is HIV-Tat, transportin 10 (TP10), or TP2.

2. The method of claim 1, wherein the NPC1 gene is under the control of a promoter, and the promoter is a truncated EF1α promoter (EF1ₜ promoter), a mini EF1α promoter (EF1α promoter), or a short EF1α promoter (EF1α S).

3. The method of claim 1, wherein the nucleic acid construct comprises a nucleic acid sequence selected from SEQ ID NOs: 1-6 and 8.

4. The method of claim 1, wherein the nucleic acid construct further comprises Intron S.

5. The method of claim 4, wherein the NPC1 gene is under the control of a promoter, and the Intron S is positioned between the promoter and the NPC1 gene.

6. The method of claim 4, wherein the Intron S comprises the nucleotide sequence of SEQ ID NO: 25.

7. The method of claim 1, wherein the nucleic acid construct comprises an expression cassette comprising a nucleotide sequence selected from SEQ ID NOs: 26-31 and 33-38.

8. The method of claim 1, wherein the nucleic acid construct further comprises first and second adeno-associated virus (AAV) inverted terminal repeats (ITRs).

9. The method of claim 1, wherein the nucleic acid construct further comprises a nucleotide sequence encoding an antibiotic resistance marker.

10. The method of claim 1, wherein the nucleic acid construct is comprised in an expression vector.

11. The method of claim 10, wherein the expression vector expresses a therapeutic amount of NPC1 in a cell that comprises the expression vector.

12. The method of claim 11, wherein the cell that comprises the expression vector is a neuronal cell.

13. The method of claim 11, wherein the NPC1-PTD fusion protein cross-corrects non-transformed cells that neighbor the cell that comprises the expression vector.

14. The method of claim 13, wherein the non-transformed neighboring cells are neuronal cells.

15. The method of claim 1, comprising administering to the subject one or more isolated or purified cell(s) comprising an expression vector comprising the nucleic acid construct.

16. The method of claim 10, wherein the expression vector is a retroviral, lentiviral, adenoviral, or adeno-associated viral (AAV) expression vector.

17. The method of claim 10, wherein the expression vector is an AAV expression vector of serotype PHP.B, Anc80, or rh10.

18. The method of claim 10, wherein the expression vector is AAV1, AAV2, AAV3, AAV4, AAV5, AAV6, AAV7, AAV8, AAV9, AAVrh8, AAVrh10, AAVrh33, AAV rh34, AAV Anc80, AAV PHP.B, AAV2/Anc80, or AAV2/PHP.B.


SEQUENCE LISTING (52 sequences)

SEQ ID NO: 1 - DNA, 3847 bp, synthetic construct
  misc_feature 1..3847: note = Synthetic
  source 1..3847: mol_type = other DNA
atgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccagattttctacttccggatgtacctggcgatggtgcttctgggagcaacccacggcctgatcttcctgcccgtgctcttgtcctacatcggccccagcgtgaacaaggccaagtcctgcgccactgaggaacgctacaagggcaccgaaagagaaaggctgctgaatttctgatagatgatag

SEQ ID NO: 2 - DNA, 3876 bp, synthetic construct
  misc_feature 1..3876: note = Synthetic
  source 1..3876: mol_type = other DNA
atgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgcttctgtcctacatcggcccgtccgtgaacaaagcgaagtcctgtgctactgaggagcggtacaagggaactgagcgcgagcgcctgctcaactttgggggcggccgcaagaagcgccggcagcgcagaaggtgatag

SEQ ID NO: 3 - DNA, 3925 bp, synthetic construct
  misc_feature 1..3925: note = Synthetic
  source 1..3925: mol_type = other DNA
atgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggaaccgagagggagcgccttctcaacttcggaggagattataaagacgatgatgacaagggggactacaaggacgacgatgacaagggcgattacaaagacgacgacgacaagttgatag

SEQ ID NO: 4 - DNA, 3910 bp, synthetic construct
  misc_feature 1..3910: note = Synthetic
  source 1..3910: mol_type = other DNA
atgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaaggggactgaacgcgaacggctgctgaactttggcggcgctgggtacctgctcggaaagatcaacctcaaagcgctggcggccctggccaagaagatcctgttgatag

SEQ ID NO: 5 - DNA, 3883 bp, synthetic construct
  misc_feature 1..3883: note = Synthetic
  source 1..3883: mol_type = other DNA
atgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggtactgaaagagagcggctgctcaacttcggtggccccctgatctacctgcgcctcctgcggggtcagttcttgatag

SEQ ID NO: 6 - DNA, 3958 bp, synthetic construct
  misc_feature 1..3958: note = Synthetic
  source 1..3958: mol_type = other DNA
atgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggaaccgagagggagcgccttctcaacttcggaggagattataaagacgatgatgacaagggggactacaaggacgacgatgacaagggcgattacaaagacgacgacgacaagggcggccggaagaagcgccgccagcggcggagattgatag

SEQ ID NO: 7 - DNA, 3994 bp, synthetic construct
  misc_feature 1..3994: note = Synthetic
  source 1..3994: mol_type = other DNA
atgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggaaccgagagggagcgccttctcaacttcggaggagattataaagacgatgatgacaagggggactacaaggacgacgatgacaagggcgattacaaagacgacgacgacaagggcggcgccggctacctcctggggaagattaacctgaaggccctggcagccctcgccaagaagatcctgttgatag

SEQ ID NO: 8 - DNA, 3967 bp, synthetic construct
  misc_feature 1..3967: note = Synthetic
  source 1..3967: mol_type = other DNA
atgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggaaccgagagggagcgccttctcaacttcggaggagattataaagacgatgatgacaagggggactacaaggacgacgatgacaagggcgattacaaagacgacgacgacaaggggggcccgctcatctacctccggctgctgcggggccagtttttgatag

SEQ ID NO: 9 - DNA, 3837 bp, Homo sapiens
  source 1..3837: mol_type = genomic DNA
atgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatcaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctag

SEQ ID NO: 10 - DNA, 247 bp, synthetic construct
  misc_feature 1..247: note = Synthetic
  source 1..247: mol_type = other DNA
agagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattc

SEQ ID NO: 11 - DNA, 4139 bp, Homo sapiens
  source 1..4139: mol_type = genomic DNA
tggctgttcctgaggcctggcctggctccccgctgaccccttcccagacctgggatggcggaggccggcctgaggggctggctgctgtgggccctgctcctgcgcttggcccagagtgagccttacacaaccatccaccagcctggctactgcgccttctatgacgaatgtgggaagaacccagagctgtctggaagcctcatgacactctccaacgtgtcctgcctgtccaacacgccggcccgcaagatcacaggtgatcacctgatcctattacagaagatctgcccccgcctctacaccggccccaacacccaagcctgctgctccgccaagcagctggtatcactggaagcgagtctgtcgatcaccaaggccctcctcacccgctgcccagcctgctctgacaattttgtgaacctgcactgccacaacacgtgcagccccaatcagagcctcttcatcaatgtgacccgcgtggcccagctaggggctggacaactcccagctgtggtggcctatgaggccttctaccagcatagctttgccgagcagagctatgactcctgcagccgtgtgcgcgtccctgcagctgccacgctggctgtgggcaccatgtgtggcgtgtatggctctgccctttgcaatgcccagcgctggctcaacttccagggagacacaggcaatggtctggccccactggacatcaccttccacctcttggagcctggccaggccgtggggagtgggattcagcctctgaatgagggggttgcacgttgcaatgagtcccaaggtgacgacgtggcgacctgctcctgccaagactgtgctgcatcctgtcctgccatagcccgcccccaggccctcgactccaccttctacctgggccagatgccgggcagtctggtcctcatcatcatcctctgctctgtcttcgctgtggtcaccatcctgcttgtgggattccgtgtggcccccgccagggacaaaagcaagatggtggaccccaagaagggcaccagcctctctgacaagctcagcttctccacccacaccctccttggccagttcttccagggctggggcacgtgggtggcttcgtggcctctgaccatcttggtgctatctgtcatcccggtggtggccttggcagcgggcctggtctttacagaactcactacggaccccgtggagctgtggtcggcccccaacagccaagcccggagtgagaaagctttccatgaccagcatttcggccccttcttccgaaccaaccaggtgatcctgacggctcctaaccggtccagctacaggtatgactctctgctgctggggcccaagaacttcagcggaatcctggacctggacttgctgctggagctgctagagctgcaggagaggctgcggcacctccaggtatggtcgcccgaagcacagcgcaacatctccctgcaggacatctgctacgcccccctcaatccggacaataccagtctctacgactgctgcatcaacagcctcctgcagtatttccagaacaaccgcacgctcctgctgctcacagccaaccagacactgatggggcagacctcccaagtcgactggaaggaccattttctgtactgtgccaatgccccgctcaccttcaaggatggcacagccctggccctgagctgcatggctgactacggggcccctgtcttccccttccttgccattggggggtacaaaggaaaggactattctgaggcagaggccctgatcatgacgttctccctcaacaattaccctgccggggacccccgtctggcccaggccaagctgtgggaggaggccttcttagaggaaatgcgagccttccagcgtcggatggctggcatgttccaggtcacgttcatggctgagcgctctctggaagacgagatcaatcgcaccacagctgaagacctgcccatctttgccaccagctacattgtcatattcctgtacatctctctggccctgggcagctattccagctggagccgagtgatggtggactccaaggccacgctgggcctcggcggggtggccgtggtcctgggagcagtcatggctgccatgggcttcttctcctacttgggtatccgctcctccctggtcatcctgcaagtggttcctttcctggtgctgtccgtgggggctgataacatcttcatctttgttctcgagtaccagaggctgccccggaggcctggggagccacgagaggtccacattgggcgagccctaggcagggtggctcccagcatgctgttgtgcagcctctctgaggccatctgcttcttcctaggggccctgacccccatgccagctgtgcggacctttgccctgacctctggccttgcagtgatccttgacttcctcctgcagatgtcagcctttgtggccctgctctccctggacagcaagaggcaggaggcctcccggttggacgtctgctgctgtgtcaagccccaggagctgcccccgcctggccagggagaggggctcctgcttggcttcttccaaaaggcttatgcccccttcctgctgcactggatcactcgaggtgttgtgctgctgctgtttctcgccctgttcggagtgagcctctactccatgtgccacatcagcgtgggactggaccaggagctggccctgcccaaggactcgtacctgcttgactatttcctctttctgaaccgctacttcgaggtgggggccccggtgtactttgttaccaccttgggctacaacttctccagcgaggctgggatgaatgccatctgctccagtgcaggctgcaacaacttctccttcacccagaagatccagtatgccacagagttccctgagcagtcttacctggccatccctgcctcctcctgggtggatgacttcattgactggctgaccccgtcctcctgctgccgcctttatatatctggccccaataaggacaagttctgcccctcgaccgtcaactctctgaactgcctaaagaactgcatgagcatcacgatgggctctgtgaggccctcggtggagcagttccataagtatcttccctggttcctgaacgaccggcccaacatcaaatgtcccaaaggcggcctggcagcatacagcacctctgtgaacttgacttcagatggccaggttttagcctccaggttcatggcctatcacaagcccctgaaaaactcacaggattacacagaagctctgcgggcagctcgagagctggcagccaacatcactgctgacctgcggaaagtgcctggaacagacccggcttttgaggtcttcccctacacgatcaccaatgtgttttatgagcagtacctgaccatcctccctgaggggctcttcatgctcagcctctgccttgtgcccaccttcgctgtctcctgcctcctgctgggcctggacctgcgctccggcctcctcaacctgctctccattgtcatgatcctcgtggacactgtcggcttcatggccctgtggggcatcagttacaatgctgtgtccctcatcaacctggtctcggcggtgggcatgtctgtggagtttgtgtcccacattacccgctcctttgccatcagcaccaagcccacctggctggagagggccaaagaggccaccatctctatgggaagtgcggtgtttgcaggtgtggccatgaccaacctgcctggcatccttgtcctgggcctcgccaaggcccagctcattcagatcttcttcttccgcctcaacctcctgatcactctgctgggcctgctgcatggcttggtcttcctgcccgtcatcctcagctatgtggggcctgacgtcaacccggctctggcactggagcagaagcgggctgaggaggcggtggcagcagtcatggtggcctcttgcccaaatcacccctcccgagtctccacagctgacaacatctatgtcaaccacagctttgaaggttctatcaaaggtgctggtgccatcagcaacttcttgcccaacaatgggcggcagttctgatacagccagaggccctgtctaggctctatggccctgaaccaaagggttatggggatcttccttgtgactgccccttgacacacgcc

SEQ ID NO: 12 - DNA, 4001 bp, Homo sapiens
  source 1..4001: mol_type = genomic DNA
tggctgttcctgaggcctggcctggctccccgctgaccccttcccagacctgggatggcggaggccggcctgaggggctggctgctgtgggccctgctcctgcgcttggcccagagtgagccttacacaaccatccaccagcctggctactgcgccttctatgacgaatgtgggaagaacccagagctgtctggaagcctcatgacactctccaacgtgtcctgcctgtccaacacgccggcccgcaagatcacaggtgatcacctgatcctattacagaagatctgcccccgcctctacaccggccccaacacccaagcctgctgctccgccaagcagctggtatcactggaagcgagtctgtcgatcaccaaggccctcctcacccgctgcccagcctgctctgacaattttgtgaacctgcactgccacaacacgtgcagccccaatcagagcctcttcatcaatgtgacccgcgtggcccagctaggggctggacaactcccagctgtggtggcctatgaggccttctaccagcatagctttgccgagcagagctatgactcctgcagccgtgtgcgcgtccctgcagctgccacgctggctgtgggcaccatgtgtggcgtgtatggctctgccctttgcaatgcccagcgctggctcaacttccagggagacacaggcaatggtctggccccactggacatcaccttccacctcttggagcctggccaggccgtggggagtgggattcagcctctgaatgagggggttgcacgttgcaatgagtcccaaggtgacgacgtggcgacctgctcctgccaagactgtgctgcatcctgtcctgccatagcccgcccccaggccctggactccaccttctacctgggccagatgccgggcagtctggtcctcatcatcatcctctgctctgtcttcgctgtggtcaccatcctgcttgtgggattccgtgtggcccccgccagggacaaaagcaagatggtggaccccaagaagggcaccagcctctctgacaagctcagcttctccacccacaccctccttggccagttcttccagggctggggcacgtgggtggcttcgtggcctctgaccatcttggtgctatctgtcatcccggtggtggccttggcagcgggcctggtctttacagaactcactacggaccccgtggagctgtggtcggcccccaacagccaagcccggagtgagaaagctttccatgaccagcatttcggccccttcttccgaaccaaccaggtgatcctgacggctcctaaccggtccagctacaggtatgactctctgctgctggggcccaagaacttcagcggaatcctggacctggacttgctgctggagctgctagagctgcaggagaggctgcggcacctccaggtatggtcgcccgaagcacagcgcaacatctccctgcaggacatctgctacgcccccctcaatccggacaataccagtctctacgactgctgcatcaacagcctcctgcagtatttccagaacaaccgcacgctcctgctgctcacagccaaccagacactgatggggcagacctcccaagtcgactggaaggaccattttctgtactgtgccaatgccccgctcaccttcaaggatggcacagccctggccctgagctgcatggctgactacggggcccctgtcttccccttccttgccattggggggtacaaaggaaaggactattctgaggcagaggccctgatcatgacgttctccctcaacaattaccctgccggggacccccgtctggcccaggccaagctgtgggaggaggccttcttagaggaaatgcgagccttccagcgtcggatggctggcatgttccaggtcacgttcatggctgagcgctctctggaagacgagatcaatcgcaccacagctgaagacctgcccatctttgccaccagctacattgtcatattcctgtacatctctctggccctgggcagctattccagctggagccgagtgatggtggactccaaggccacgctgggcctcggcggggtggccgtggtcctgggagcagtcatggctgccatgggcttcttctcctacttgggtatccgctcctccctggtcatcctgcaagtggttcctttcctggtgctgtccgtgggggctgataacatcttcatctttgttctcgagtaccagaggctgccccggaggcctggggagccacgagaggtccacattgggcgagccctaggcagggtggctcccagcatgctgttgtgcagcctctctgaggccatctgcttcttcctaggggccctgacccccatgccagctgtgcggacctttgccctgacctctggccttgcagtgatccttgacttcctcctgcagatgtcagcctttgtggccctgctctccctggacagcaagaggcaggaggcctcccggttggacgtctgctgctgtgtcaagccccaggagctgcccccgcctggccagggagaggggctcctgcttggcttcttccaaaaggcttatgcccccttcctgctgcactggatcactcgaggtgttgtggtgggggccccggtgtactttgttaccaccttgggctacaacttctccagcgaggctgggatgaatgccatctgctccagtgcaggctgcaacaacttctccttcacccagaagatccagtatgccacagagttccctgagcagtcttacctggccatccctgcctcctcctgggtggatgacttcattgactggctgaccccgtcctcctgctgccgcctttatatatctggccccaataaggacaagttctgcccctcgaccgtcaactctctgaactgcctaaagaactgcatgagcatcacgatgggctctgtgaggccctcggtggagcagttccataagtatcttccctggttcctgaacgaccggcccaacatcaaatgtcccaaaggcggcctggcagcatacagcacctctgtgaacttgacttcagatggccaggttttagcctccaggttcatggcctatcacaagcccctgaaaaactcacaggattacacagaagctctgcgggcagctcgagagctggcagccaacatcactgctgacctgcggaaagtgcctggaacagacccggcttttgaggtcttcccctacacgatcaccaatgtgttttatgagcagtacctgaccatcctccctgaggggctcttcatgctcagcctctgccttgtgcccaccttcgctgtctcctgcctcctgctgggcctggacctgcgctccggcctcctcaacctgctctccattgtcatgatcctcgtggacactgtcggcttcatggccctgtggggcatcagttacaatgctgtgtccctcatcaacctggtctcggcggtgggcatgtctgtggagtttgtgtcccacattacccgctcctttgccatcagcaccaagcccacctggctggagagggccaaagaggccaccatctctatgggaagtgcggtgtttgcaggtgtggccatgaccaacctgcctggcatccttgtcctgggcctcgccaaggcccagctcattcagatcttcttcttccgcctcaacctcctgatcactctgctgggcctgctgcatggcttggtcttcctgcccgtcatcctcagctacgtggggcctgacgttaacccggctctggcactggagcagaagcgggctgaggaggcggtggcagcagtcatggtggcctcttgcccaaatcacccctcccgagtctccacagctgacaacatctatgtcaaccacagctttgaaggttctatcaaaggtgctggtgccatcagcaacttcttgcccaacaatgggcggcagttctgatacagccagaggccctgtctaggctctatggccctgaaccaaagggttatggggatcttccttgtgactgccccttgacacacgcc

SEQ ID NO: 13 - DNA, 4360 bp, Felis catus
  source 1..4360: mol_type = genomic DNA
caccaaggcagcatgaccgcgcgcggcccagccctcggcctccttctgctgctgctgtgtcccgcgcaggtgttggcacagtcttgcatctggtatggagagtgtggaattgcatctggagataagaggtacaactgcaaatattcagggccaccaaaacccttgccgaaggatgggtatgacttagtgcaggagctctgtccaggattcttctttgacaatgtcagtctttgttgcgatgtgcagcagctccagacactgaaagacaacctgcagctgcctctacagtttctgtccagatgtccatcctgtttctataacctagtgaacctgttttgcgagctgacgtgtagccctcggcaaagtcagtttctgaatgttacagcaactgaagattatgttgatcctgttacaaaccagactaaaacaaatgtaaaagaattacagtactacattggagagagttttgccaatgccatgtacaatgcctgccgggacgtggaggccccctccagtaatgacaaagccctgggactgctgtgtgggaaggacgccgaagcctgcaatgccactaactggattgagtacatgttcagtaaggacaacggccaggcacctttcaccatcacacccattttttcagatcttccaacccatgggatggagcccatgaacaatgccaccaagggctgtgacgagtctgtggacgaggtcaccgggccgtgcagctgccaggactgctcaattgtttgtggcccaaagccccagcccccaccgcctcctgtgccctggagaatcttgggcctggacgccatgtatgtcatcatgtggatcacctacatggcatttttgcttgtgttttttggagctttttttgctttgtggtgctacagaaaacgctattttgtctccgagtacacccccattgatagcaacatagctttctctgtaaatgccaatgacagaggggaggcgtcctgctgcgacgcgcttggtgcggcatttgagggctgtctgaggcggctcttctcacagtggggctctttctgcgtccgaaacccgggccccatcatcttcttctccctggccttcattgccgcctgttcttcaggcctggtgtttgtgcgggtcacgaccaatccagttgacctgtggtcagctcccagcagccaggcgcgcctcgagaaagagtactttgacacgcactttgggcctttcttccgcacggagcagctcatcatccaggccccccacaccagtgcacacacttaccagccgtacccctcaggatccgatgtgcccttcggacctccgcttgacctcgcgatcttgcaccaggttcttgacttacaaactgccatcgaaaatatcactgcatcttataacaatgagaccgtgacacttcaagacatctgcgtggcccccctgtcaccctataacaagaactgcaccattctgagcgtgttaaattacttccagaacagccattccatgctggaccatgaaataggagatgacttctttgtgtatgcagattaccacacgcacttgctatactgtgtacgggctcctgcctctctgaacgataccagtttgctccatgatccttgcctgggtacattcggtgggccagtgttcccgtggcttgtgttaggaggctatgatgatcaaaattacaataatgccacagcccttgtgattacctttcctgtcaataattactataatgatacagagaggctccagaaggcccacgtctgggaaaaagagtttattaattttgtgaaaaactacaagaatccaaatctgaccatttctttcactactgagcgaagtattgaagatgaactaaatcgtgaaagtaacggtgatattttcactgttataatcagctatgccatcatgtttctgtacatttccatagccttggggcacatcaaaagctgtagcaggcttctagtggactctaaaatctccctcggcatcgcggggatcctcattgtgttgagctcaaaggcgtgctcgttgggcatctttagttacgttgggatccccctcaccctcattgtgatcgaagtcatcccattcctggtgctggctgttggggtggacaacatcttcattctggtccagacctaccagcgagatgaacgtcttcatggagaaactctggatcagcagctgggcagggtcctaggagaagtggctcctagtatgttcctgtcatccttttcagaggctgtagcatttttcttaggagcgctgtcaaagatgccggctgttcacaccttctctctgtttgccgggatggcagtcctcatcgacttccttcttcagattacctgtttcgtgagtctcttggggttagacattaagcgtcaagagaaaaaccgtctggatgttctttgctgtgtcagaggctctgaagatggaaccagtgtccaggcttcagagagctgcttgtttcggctcttcaagcactcctattctccacttctgcttaaggactggatgagaccaattgtgatagcaatatttgtgggtgtcctgtcattcagtgttgcagtcctgaacaaagtggaaatcggattggatcagtctctttcaatgccagatgactcctacgtgatggattatttcaagtccctcaaatacctgcatgcaggtccacctgtgtactttgtcctggaggaagggcatgactacacctctctgaaagggcagaacatggtgtgcggaggcatgggctgcaataacgactccctggtgcagcagatattcaacgcggcccagctagacagctatacccgaataggctttgctccctcttcctggatcgacgattactttgattgggtcaagcctcagtcttcttgctgtagagtctacaacagcaccgatcggttctgcaatgcttcagtggttgaccctgcctgcatccgctgcaggcccctcacccaggagggcaaacagaggcctcaaggtggagacttcatgagattcctgcccatgttcctttctgataacccgaaccccaagtgcggcaaagggggacatgctgcttacagttcggcagttaacatcctcggcaatgacacgggtgtcggagccacttacttcatgacctaccacaccgtgcttcagacgtctgctgactttactgacgccatgagaaaagccaacctcatcgccagtaacatcaccaaaaccatgggccttgaaggaagtaattaccgtgtgttcccatacagtgtgttctatgtcttctacgaacagtacctgaccattattgatgacacgatctttaacctcagcgtgtccctgggagccatcttcttagtgaccgtgattctcctgggctgtgatctgtggtctgcagtgatcatgtgtatcaccatcgctatgatcttggtcaacatgtttggcgtcatgtggctgtggggcatcagtctgaatgcagtttccttggtcaacttggttatgagctgtggcatttccgtggagttctgcagccacataacgagagcattcacagtgagcatgaagggcagccgtgcacaacgggcagaagaggcgctcgctcacatgggcagttctgtgttcagtggaatcacactaacaaaatttggagggattgtggtattggcctttgccaaatctcagattttccagatattttacttcaggatgtatttggctatggtcttactgggagccacccacggcttgatcttcctcccggtcttactcagctacataggcccatcaataaataaagccaaaagcttggccactcaagagcaatacaaaggtacagagcgagaacaactcctaaatttctagccctcttgcaaggtcgggggactctgaactgtgtctgtgggtcgctcagcctaccgctcactggacagtggctgcaccgtcgaggccgagttggacactggacggtaccagacatcaggctacttaacagcagcagcttcggccatagtgcttttaaactcaggaatgcaaatgtgatgcgtgaatcggaagcattacggaatcggaaggcagccccgggacgctgcagggccttccagttctccacgaaccagacctaactcttgggcaggcggaaggtgacactagcatggctgagtgggatctgctccgtgacactttttaaaggccaatcaatgcaatgtttttccttttgtttgggagtaagccatcacacagattctataccgtatttttagtaacatttgaggatggtgtagatacactttactataacattttgtagtttaaagagctttattaatgcaataaattaactttgtacacatttttatatatatataaaaaaactagcaag

SEQ ID NO: 14 - DNA, 5016 bp, Mus musculus
  source 1..5016: mol_type = genomic DNA
ccttcctgccttgcgcacacagtccgccgtctgctcttgccccctccttggtcaggcgccggttccgaaaccgcccgccagtgccgcgacgctcgggtcgcggtgctccgcgagccgaactgagagctgtagccccgcgcggcgacagcatgggtgcgcaccacccggccctcggcctgctgctgctgctgctgtgccctgcgcaggtgttttcgcaatcctgtgtttggtatggagagtgtggaattgcgactggagataagaggtacaactgtaaatattctggcccaccaaaacccctcccaaaggacggctatgacttagtgcaggaactctgtccaggactcttctttgacaatgtcagtctctgctgtgacattcaacagcttcagacgctgaagagtaacctgcagctgcccctgcagttcctgtccaggtgtccgtcatgtttttataacctaatgaccctgttttgtgagctaacatgtagcccacaccagagtcagtttctgaatgtgacagcaactgaggattattttgatcctaagacacaggagaataaaacaaatgtaaaggaattagagtactttgtcggacagagcttcgcgaatgcgatgtacaatgcctgccgtgatgtggaggcgccttccagtaacgagaaggccttaggactcttgtgtgggagggatgcccgtgcctgcaatgccaccaactggattgagtacatgttcaataaagacaacggacaagcgccatttaccatcattcctgtgttttcagatctttcaatccttgggatggagcccatgagaaatgccaccaaaggctgcaatgagtctgtagatgaggtcacggggccatgtagctgccaggactgctccatcgtctgcggccccaagccccagcccccaccccctcctatgccctggaggatctggggcttggatgccatgtatgtcatcatgtgggtcacctacgtggcatttctgtttgtgttttttggagcactgttggcagtgtggtgccacagaaggcggtactttgtgtctgagtacactcccattgacagtaacatcgccttttctgtgaatagcagtgacaaaggggaagcctcatgctgtgacccacttggtgcagcatttgatgactgtctgaggcgcatgttcacaaagtggggggctttctgtgtccgaaatcccacctgcatcattttcttctcattggccttcatcactgtgtgctcttctggcctggtatttgtccaggtcaccaccaatcctgtagagctctggtcagcccctcacagtcaggcccgcttggaaaaggagtactttgacaagcactttgggcctttctttcgcacggagcagcttatcatccaggcccccaacaccagtgttcatatctacgaaccgtaccccgcaggagccgatgtgcccttcgggcctccattgaacaaagagattctgcaccaggttctggacttacagatcgccattgaaagcatcaccgcatcttacaacaatgaaaccgtgacactgcaggacatctgtgtggcccccctctctccatacaacaagaactgcaccattatgagtgtgttaaattacttccagaacagccatgcggtgctggacagccaagtaggcgacgacttctatatctacgctgattaccacacacactttctgtactgtgtacgggctcccgcctccttgaatgatacgagtttgctccacggtccttgcctgggtacatttggaggaccggtgttcccgtggcttgtgttgggtggctatgatgatcagaactacaacaatgccaccgcgcttgtgatcaccttccccgtgaataattactacaatgacacagagaggctccagagggcctgggcctgggagaaagagtttattagttttgtgaaaaactacaagaatccaaatctgaccatttctttcactgctgagcgaagcatcgaagatgagctcaatcgggaaagtaacagtgacgtgttcaccgtcatcatcagctacgtcgtgatgtttctgtacatttccctcgccctgggtcacatccagagctgcagcaggctcctggtggattctaagatctcgctgggcattgcggggatcctgatcgtgctaagctcggtggcctgctctctgggcatcttcagctacatggggatgccgctgaccctcatcgtcattgaggtcatcccattcctggtgctggctgtcggggtggacaacatcttcattctagtgcagacctaccagagagatgagcgtcttcaggaggaaacgctggatcagcagctgggcaggatccttggagaagtggccccgaccatgttcctttcatccttttctgagacctcagcatttttctttggggcgctgtcctcgatgccagctgtgcacaccttctctctgtttgcgggaatggccgtcctcattgacttcctccttcagattacctgctttgtgagcctgttggggttagatattaagaggcaagagaaaaaccatctggacatcctgtgctgtgtcagaggcgctgacgacggacaaggtagccacgcctccgaaagctacctgtttcgcttcttcaaaaactactttgccccccttctgctgaaggactggctgaggccaattgtggtagcggtgtttgtgggcgttctgtcattcagtgttgcggtggtgaacaaagtagacatcgggttggatcagtctctctcaatgccaaacgattcgtatgtgattgactatttcaaatcactcgctcagtacctgcactcgggcccacccgtgtactttgtcctggaggaaggctataactacagttcacgcaaagggcagaacatggtgtgcggcggcatgggctgtgacaatgactccctggtgcagcagatatttaacgcagctgagctggacacctacacccgagtaggcttcgccccctcgtcctggatcgatgactactttgactgggtctcgccacagtcgtcctgctgcagactctacaacgtcactcaccagttctgcaatgcttctgtgatggacccaacctgtgtccgctgcagacctctgactccagagggtaaacagaggcctcaggggaaagaattcatgaaattcctgcccatgttcctttctgataaccccaaccccaagtgcggcaaagggggacatgctgcttacggttcagctgttaacattgtgggagatgacacttacattggggccacttacttcatgacctaccacaccatacttaagacctccgctgactatactgatgccatgaagaaagctcggctaatagccagtaacatcacggaaaccatgcgttctaaggggagtgactaccgcgtattcccttacagtgtgttctacgtcttctatgaacagtacctgaccattattgatgacaccatctttaacctcagtgtgtctctgggctccatatttctggtgaccttggtggttctgggctgtgagctgtggtctgcggtcatcatgtgtatcaccatagccatgatcctggtcaacatgttcggtgtcatgtggctgtggggcatcagtctgaatgcggtctccttggtcaacttggtgatgagctgcggcatttctgtggagttctgcagccatataacgagagcattcaccatgagtaccaaaggaagccgagtgagccgggcggaagaggcactggcccacatgggtagttctgtattcagtggaatcacacttacgaaatttggagggatcgtggtgttagcctttgccaaatctcaaatttttgagatattttacttcaggatgtacttagccatggtcttactcggagccactcatggactaatatttcttcccgtcttactcagttacatagggccgtcggtgaataaagctaaaagacacaccacttacgagcgctacagagggacagagagaaaacggctcctcaatttttagccttgtagcaggctttggtgactgtgtttatggataggtcaagtttactgcaagacagctgcgctgtcaagactgagctggcttcaggctgtgtccgagctgtgtcacatgcagctctacccacgcttttaaactcaggaatgcacacctaacttgtgaagcagtattaatggatctgaaagcaacaatcgccagcccctactgtcgtaccagtagaaacctcatcttgggtacaaggaaggatagttctgtcactttaacttgtttcaatgcctacttttaatggaggttattaaacactttaacctcccttctagcccaccaccaaccagagatagtgggaaagaaaggatacaggggaagtggacctgtttagaaatggttctttggagcagatcctgtctgcattatcaggaaaccagcaattcagttcacgggtcagcagtggcagctcgacccactcgcaaacacttcacggatacaccagcagtgttgggataggagcagccaggcctcagcaggagggaccagggccgacaggaacaccagaggttcttggctgttcctctatcagcgaagactggagaccaacaaacattacacagctagctctatattctctctgtggagtcccaacacatggagctcaactacacaatataaggcagaccaaccaatacatgcctgtcattcacgtgtcctttcatgtgcttgctttagggaaacagtccttcacaagtctgcctttcacctgtgtctgcttcagcaaaatgttctttcacatttgccccagcaaaaccccatctaacacaactgactttccaaagaacccttaagtttccatttcccagtggcaataactgtgacctgatccctaccccacatgctgtctccttttctttgggagttagcaacatttgaggatgttgtaggtactttattacatttttttgtagtttaaagagctttattaatgcaataaattaactttgtacatttttatattaaaaaaaaaaaaaaaaaaaaaaaaaaa

SEQ ID NO: 15 - DNA, 4724 bp, Bos taurus
  source 1..4724: mol_type = genomic DNA
gagccggcgcttggtttcctccctccgcgggcgcgggagccggagacagcccggggtgcccaaccgagcctgcgagccgagtctctccgacatccgccgcccgtcgccgcggcagcatgagcgtgcgcggcccagccttcggcctcctcctgctgctgttctgcccggcgcaggtattttcacagtcctgcatttggtatggagagtgtggaattgcatctggagataagagatacaactgcagatattctgggccaccagagccattgccacaggatggctatgacctagtgcaggaactctgtccagggttcttctttggcaatgtcagtctttgctgtgatgtccagcagcttcacacactgaaggacaacctgcagctgcctctgcagtttctgtccagatgtccatcctgtttttataacctagtgaacctgttttgtgagctgacatgtagccctcgacaaagtcagtttctgaacgttacagcaactgaagattatgttgatcctgctacaaaccagacgaaaacaaatgtaaaagaattacagtactatgttggagagagttttgccaatgccatgtacaattcctgccgggatgtggaggccccctcgagcaacgagaaggccctggggctgctgtgcgggcgtgaggccagcgcctgcaatgctactaactggatcgagtacatgttcaacaaggacaatggccaggcgcccttcaccatcacccccgtcttttcagatcttccgacgcatgggatggagcccatgaacaatgccaccaagggctgtgacgagtcggtggacgaggtcacggggccgtgcagctgccaggactgctcggccgtctgcggccccaagccccagcccccaccccctcccgttccctggagaatcctgggtctggacgccatgtatgtcatcatgtggagcacctacatggctttcctgctcgtgttctttggagcgttctttgctgtgtggtgctacagaaaacggtattttgtctctgagtttacccccattgatggcaatatacctttctctataaacgccagtgacaaaggggggccaacctgctgtgaccctctgggtgccgccttcgaggctcatctgcggcggctcttcgaatggtggggctccttctgcgtgaggcaccccggctgtgtcgtgttcttctccgtggccttcatcgccgcctgttcctcaggcctggtgttcatccaggtcacaaccgacccggtggacctctggtcggcccccggcagccaggcgcgcctggagaaggagtactttgacacgcacttcgggcccttcttccgcaccgagcagctcatcatcagggccccccacaccccgccgcacatatacgagccctacccctcgggagccgacgtgccctttgggcctccccttgctgtagacattctgcaccaggtccttgacttacaaaccgccatcgagagcatcactgcttcttacaacaacgagactgtgaccctgcgggacatctgcgtggcgcccctctcgccatacaaccagaactgcaccatcctgagtgtgctgaactacttccagaacagccactctgtgctggaccatcaagtcggggatgacttcttcgtgtatgcagattaccacacgcacttcctctactgtgtccgggctcctgcttccctgaatgacaccagtttgctccacgatccttgcctgggaacgtttggtggcccagtgttcccatggcttgtgcttggaggctacgatgatcaaaactacaataacgcaacagcccttgtgatcacctttcctgtcaataattactataacgatacagagaagctccagagggcccaggcctgggaaagagagttcattaattttgtgcaaaactacgagaatccaaatctgaccatttcttttaaggctgaacgaagcattgaagatgaactaaaccgtgaaagtaacagtgatgtgttcactgttctgatcagctatggcgtcatgtttttatatatttccatagccttggggcatatcaaaagctgtcgcaggcttctggtggattcgaaaatctccctgggcattgccggtgtcctgattgtgttgagctcagtggcctgctccttgggcatcttcagctacatcggggtccccctcaccctcattgtgatagaagtcatcccattcctcgtgctggctgtgggggtggacaacatcttcatcctggtccagacctaccagagagatgaacgtcttcaaggggaaaccctcgaccaacaggtgggccgggtcctgggagaagtggctcccagcatgttcctctcatccttcgcagagacggtagcgtttttcttaggaggcctgtctgtgatgccggccgtgcacacgttctctctcttcgcgggcatggcagtcctcattgacttcctcctgcagatcacctgcttcgtgagcctcctgggtttggacattaagcggcaagagaagaaccagctggacgttctctgctgtgtcgggggggcggcagacgacgcgggcatccaggcctccgagagctgcttgttccgcttcttcagaaactcctacgcgccgcttctgctcaaggactggatgcggcccctcgtggtggcagtatttgtgggtgtgctgtcgttcagtatcgcagtcctgaacaaagtagagatcggattggatcagtctctttcaatgccggatgactcctacgtgacggattacttccagtccctgaatcagtacctgcacgcaggcccacctgtgtacttcgtgctggaggagggccacgactacacgtccacgaaggggcagaacatggtgtgcgggggcctcggctgcaacaacgactcgctggtgcagcaggtcttcacggccgcccagctggacagctacacccggataggctttgccccgtcgtcctggattgacgattactttgactgggtcaagcctcaatcttcttgctgtcgcatctacaacagcacggagcagttctgcaatgcatcggtggtcaaccctacctgtgtccgctgccggccgctgactccagagggcaagcagagacctcagggcgcagacttcatgaggttcctgcccatgttcctctccgacaacccgaaccccaagtgtggcaaagggggacacgctgcctacagcgcagccgtcaacatcctggacaacggcacgagggtgggagccacgtacttcatgacctaccacacggtgctgcagacctcggctgacttcatcgatgccatggagaaggcccgcctcatcgccagcaacatcaccaggaccatgaaccagcaggggggtgatcaccgggtgttcccatacagcgtgttctatgtcttctatgagcagtacctgaccatgattgacgacaccatcttcaacctcagcgtgtccctgggcgccatcttcctggtggccgtggtcctcttgggctgtgagctgtggtctgcggtcatcatgtgcgccaccatcgccatgatcttggtcaacatgtttggtgtcatgtggctgtggggcatcagcctgaacgcggtttctctggtcaacctggtcatgagctgtggcatctccgtggagttctgtagccacatcacgagggccttcacggtgagcacgaagggcagccgcgtagagcgggcggaggaggcgctctcccacatgggcagctctgtattcagtggaatcacactaacgaaatttggaggcattatcgtgttggcttttgccaaatctcaaattttccagatattttacttcaggatgtatttagctatggtcttactgggagccactcatggactaatattcctccctgtcctactcagttacataggcccgtcaataaataaagccaaaagtttgaccacgcagcagcgatacagaggtacagaacgagaacagctcctcaacttctagctgccccctccctgaagtgagactgtgtctgcgggtcggttggtttacaacagcaaccgtgttgtcgaggctcggttgaaccctggacgtgagcaagcgtcaggctacttaacagcagccgctttggctgtagcgcttttaaactcaggaatgcagactctgactggaagcagcattactgaacctggaggcagccccagggcgctggggcacctttccgttccccaggaatgaggcctggttctcaggcagccagaagggggagctagggagcctgcggtccgctcactgacacttcctaaaggccaatcaatgcaatgtttctcctttttcaggagtaagccatcacacaagttctacaccttatttttagtaacatttgaggatgtggtagatacatactttattacaacattttgtagtttaaagagctttattaacgcaataaattaactttgtacacattttatatatatatatatatatatatataaaaaaaactagcaagtgacctcagaacgttgtaggcctcattagagcttggtctccgaaaacttgtttgaaaaaagcgacatgttcttcacagtgttctcctgtaaaggaaaatgcagatttcatctgttaaaacgaggcacaaaaggaggaaaacaagggaacactttgcggctcctagatgtggatactgggttttaacttatttttctctcataaaatactttgttttcctaaaaaaaaaaaaaaaaaaaaaaaaaaaaaagaaaaaaaaaaaaaaaa

SEQ ID NO: 16 - DNA, 1440 bp, Danio rerio
  source 1..1440: mol_type = genomic DNA
ggggaggaggcgttggcacatatgggcagctccgtcttcagtggaatcaccctcaccaagtttggcgggatcctgattctcgctctgtccaaatcgcagatcttccagattttctacttccgcatgtatctggcaatcgtgctgctgggagccgctcacggcctcatcttcctgcctgtcttactcagttatgcaggcccgtctgtaaataaagcaaaggttctagctgcgcacaacaggtttgtgggcacagagagagagcggctcatctactgatcctcagcggacgacgccagcttttcatattcatgttacacacatttgtaactcattcagctgacagacttaactcaggactgcgctttggatgaacagaagacctttcttccactacttctcctgtctgcgtccaccaaataaacctaacacacttggtagatcctgacatgatgcagtatatagagtttgtggagaagaaaactcttatttccattaaacacaaacctgtggttctggtaaagcaacaacgagagcatgagggcaacgtttgtttcgtttgtatgaatgtatacgtgtaaaaaaaatgtttgactaatcctttttttatttatttgtatgcaattatttacttattgttagcatgtttcacataacgagtcaaattcacagataatgaaagaaccgtgttgacaatctcttgatgtattgtgatgctttgacaagaaccgtatgttacacacagctagcgctttattgctagcatgacctgaacggtgttgttttcaagcaggaaaaccacgttagtcttagctaattaatgaaattggttgcagtgggacgttttaaaattgtttacttacaaagttgtgctgcgttaattagcatagacatgctaataaagggagtgccagcattagtttagtgacttaaggcggcaattctaaccaaaacacaagcaaatataggttgcagtttggtttaactctcatatgttcagaatttgcctgttatttcaattactaacacagtttagctttaaaatggctgctcacatggttttgtaaaccatcaatatggaaggggaattagcctactatagtaaacttagcaacgtgcaaggcagaaatgtgtttgcatacaaataaatggttatttttatgataaaagtttgggattgatagggattgtgggtatgatgtgatttttcaaagtaatgtttgtcattagtcagggactggaaacttggacatttatgtaaacatttccatagctttggtaaaagatagatgcgtgtgcctcatggacataatagcagtctaaagtttctgctttgtgcgtaaacgttcttgatttgaagatttttttttggaataaatacggactaatttgtaataaaaaagattgttttatttaaaaaaaaaaaaaaaaaaaaaaaaaaaa

SEQ ID NO: 17 - DNA, 4827 bp, Homo sapiens
  source 1..4827: mol_type = genomic DNA
gaagggcaacacggggaccttgaagcggggtcgcggcggcgccccagcccgggccagggagtcccggcagcggcacctcccagaaagggcggagccgacgacgccttcttccttcctgaccggcgcgcgcagcctgctgccgcggtcagcgcctgctcctgctcctccgctcctcctgcgcggggtgctgaaacagcccggggaagtagagccgcctccggggagcccaaccagccgaacgccgccggcgtcagcagccttgcgcggccacagcatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatgaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctagccctctcgcagggcatcctgactgaactgtgtctaagggtcggtcggtttaccactggacgggtgctgcatcggcaaggccaagttgaacaccggatggtgccaaccatcggttgtttggcagcagctttgaacgtagcgcctgtgaactcaggaatgcacagttgacttgggaagcagtattactagatctggaggcaaccacaggacactaaacttctcccagcctcttcaggaaagaaacctcattctttggcaagcaggaggtgacactagatggctgtgaatgtgatccgctcactgacactctgtaaaggccaatcaatgcactgtctgtctctccttttaggagtaagccatcccacaagttctataccatatttttagtgacagttgaggttgtagatacactttataacattttatagtttaaagagctttattaatgcaataaattaactttgtacacatttttatataaaaaaacagcaagtgatttcagaatgttgtaggcctcattagagcttggtctccaaaaatctgtttgaaaaaagcaacatgttcttcacagtgttcccctagaaaggaagagatttaattgccagttagatgtggcatgaaatgagggacaaagaaagcatctcgtaggtgtgtctactgggttttaacttatttttctttaataaaatacattgttttcctaaaaaaaaaaaaaaa

SEQ ID NO: 18 - DNA, 5209 bp, Mus musculus
  source 1..5209: mol_type = genomic DNA
ccttcctgccttgcgcacacagtccgccgtctgctcttgccccctccttggtcaggcgccggttccgaaaccgcccgccagtgccgcgacgctcgggtcgcggtgctccgcgagccgaactgagagctgtagccccgcgcggcgacagcatgggtgcgcaccacccggccctcggcctgctgctgctgctgctgtgccctgcgcaggtgttttcgcaatcctgtgtttggtatggagagtgtggaattgcgactggagataagaggtacaactgtaaatattctggcccaccaaaacccctcccaaaggacggctatgacttagtgcaggaactctgtccaggactcttctttgacaatgtcagtctctgctgtgacattcaacagcttcagacgctgaagagtaacctgcagctgcccctgcagttcctgtccaggtgtccgtcatgtttttataacctaatgaccctgttttgtgagctaacatgtagcccacaccagagtcagtttctgaatgtgacagcaactgaggattattttgatcctaagacacaggagaataaaacaaatgtaaaggaattagagtactttgtcggacagagcttcgcgaatgcgatgtacaatgcctgccgtgatgtggaggcgccttccagtaacgagaaggccttaggactcttgtgtgggagggatgcccgtgcctgcaatgccaccaactggattgagtacatgttcaataaagacaacggacaagcgccatttaccatcattcctgtgttttcagatctttcaatccttgggatggagcccatgagaaatgccaccaaaggctgcaatgagtctgtagatgaggtcacggggccatgtagctgccaggactgctccatcgtctgcggccccaagccccagcccccaccccctcctatgccctggaggatctggggcttggatgccatgtatgtcatcatgtgggtcacctacgtggcatttctgtttgtgttttttggagcactgttggcagtgtggtgccacagaaggcggtactttgtgtctgagtacactcccattgacagtaacatcgccttttctgtgaatagcagtgacaaaggggaagcctcatgctgtgacccacttggtgcagcatttgatgactgtctgaggcgcatgttcacaaagtggggggctttctgtgtccgaaatcccacctgcatcattttcttctcattggccttcatcactgtgtgctcttctggcctggtatttgtccaggtcaccaccaatcctgtagagctctggtcagcccctcacagtcaggcccgcttggaaaaggagtactttgacaagcactttgggcctttctttcgcacggagcagcttatcatccaggcccccaacaccagtgttcatatctacgaaccgtaccccgcaggagccgatgtgcccttcgggcctccattgaacaaagagattctgcaccaggttctggacttacagatcgccattgaaagcatcaccgcatcttacaacaatgaaaccgtgacactgcaggacatctgtgtggcccccctctctccatacaacaagaactgcaccattatgagtgtgttaaattacttccagaacagccatgcggtgctggacagccaagtaggcgacgacttctatatctacgctgattaccacacacactttctgtactgtgtacgggctcccgcctccttgaatgatacgagtttgctccacggtccttgcctgggtacatttggaggaccggtgttcccgtggcttgtgttgggtggctatgatgatcagaactacaacaatgccaccgcgcttgtgatcaccttccccgtgaataattactacaatgacacagagaggctccagagggcctgggcctgggagaaagagtttattagttttgtgaaaaactacaagaatccaaatctgaccatttctttcactgctgagcgaagcatcgaagatgagctcaatcgggaaagtaacagtgacgtgttcaccgtcatcatcagctacgtcgtgatgtttctgtacatttccctcgccctgggtcacatccagagctgcagcaggctcctggtggattctaagatctcgctgggcattgcggggatcctgatcgtgctaagctcggtggcctgctctctgggcatcttcagctacatggggatgccgctgaccctcatcgtcattgaggtcatcccattcctggtgctggctgtcggggtggacaacatcttcattctagtgcagacctaccagagagatgagcgtcttcaggaggaaacgctggatcagcagctgggcaggatccttggagaagtggccccgaccatgttcctttcatccttttctgagacctcagcatttttctttggggcgctgtcctcgatgccagctgtgcacaccttctctctgtttgcgggaatggccgtcctcattgacttcctccttcagattacctgctttgtgagcctgttggggttagatattaagaggcaagagaaaaaccatctggacatcctgtgctgtgtcagaggcgctgacgacggacaaggtagccacgcctccgaaagctacctgtttcgcttcttcaaaaactactttgccccccttctgctgaaggactggctgaggccaattgtggtagcggtgtttgtgggcgttctgtcattcagtgttgcggtggtgaacaaagtagacatcgggttggatcagtctctctcaatgccaaacgattcgtatgtgattgactatttcaaatcactcgctcagtacctgcactcgggcccacccgtgtactttgtcctggaggaaggctataactacagttcacgcaaagggcagaacatggtgtgcggcggcatgggctgtgacaatgactccctggtgcagcagatatttaacgcagctgagctggacacctacacccgagtaggcttcgccccctcgtcctggatcgatgactactttgactgggtctcgccacagtcgtcctgctgcagactctacaacgtcactcaccagttctgcaatgcttctgtgatggacccaacctgtgtccgctgcagacctctgactccagagggtaaacagaggcctcaggggaaagaattcatgaaattcctgcccatgttcctttctgataaccccaaccccaagtgcggcaaagggggacatgctgcttacggttcagctgttaacattgtgggagatgacacttacattggggccacttacttcatgacctaccacaccatacttaagacctccgctgactatactgatgccatgaagaaagctcggctaatagccagtaacatcacggaaaccatgcgttctaaggggagtgactaccgcgtattcccttacagtgtgttctacgtcttctatgaacagtacctgaccattattgatgacaccatctttaacctcagtgtgtctctgggctccatatttctggtgaccttggtggttctgggctgtgagctgtggtctgcggtcatcatgtgtatcaccatagccatgatcctggtcaacatgttcggtgtcatgtggctgtggggcatcagtctgaatgcggtctccttggtcaacttggtgatgagctgcggcatttctgtggagttctgcagccatataacgagagcattcaccatgagtaccaaaggaagccgagtgagccgggcggaagaggcactggcccacatgggtagttctgtattcagtggaatcacacttacgaaatttggagggatcgtggtgttagcctttgccaaatctcaaatttttgagatattttacttcaggatgtacttagccatggtcttactcggagccactcatggactaatatttcttcccgtcttactcagttacatagggccgtcggtgaataaagctaaaagacacaccacttacgagcgctacagagggacagagagagaacggctcctcaatttttagccttgtagcaggctttggtgactgtgtttatggataggtcaagtttactgcaagacagctgcgctgtcaagactgagctggcttcaggctgtgtccgagctgtgtcacatgcagctctacccacgcttttaaactcaggaatgcacacctaacttgtgaagcagtattaatggatctgaaagcaacaatcgccagcccctactgtcgtaccagtagaaacctcatcttgggtacaaggaaggatagttctgtcactttaacttgtttcaatgcctacttttaatggaggttattaaacactttaacctcccttctagcccaccaccaaccagagatagtgggaaagaaaggatacaggggaagtggacctgtttagaaatggttctttggagcagatcctgtctgcattatcaggaaaccagcaattcagttcacgggtcagcagtggcagctcgacccactcgcaaacacttcacggatacaccagcagtgttgggataggagcagccaggcctcagcaggagggaccagggccgacaggaacaccagaggttcttggctgttcctctatcagcgaagactggagaccaacaaacattacacagctagctctatattctctctgtggagtcccaacacatggagctcaactacacaatataaggcagaccaaccaatacatgcctgtcattcacgtgtcctttcatgtgcttgctttagggaaacagtccttcacaagtctgcctttcacctgtgtctgcttcagcaaaatgttctttcacatttgccccagcaaaaccccatctaacacaactgactttccaaagaacccttaagtttccatttcccagtggcaataactgtgacctgatccctaccccacatgctgtctccttttctttgggagttagcaacatttgaggatgttgtaggtactttattacatttttttgtagtttaaagagctttattaatgcaataaattaactttgtacatttttatattaaaaaaaaaaaaagactattaagggacttcagaatgttgtaggcctcattagagcttgttctccaaaaacctgcttgaaaaaggcaacgtgttcttcacagtgctctcctgtaaggacaacgcggctcagtcagcagctggttatggcccgggaagacaggatgcagacaacgctcatgggttttaacttatttttctttaataaaatacctcctcctcc

SEQ ID NO: 19 - DNA, 1033 bp, Homo sapiens
  source 1..1033: mol_type = genomic DNA
ggtccgggcgggtcaggcctctgaggggatggcatgggcggagactgcaggcttctgggcctgagagggctggagctggctttccggagccgggggcggggcgcgggcgggcctcagctgtggttactggtgacaggtcgcctgactgggctcctccccgggcccgccccgacaggtttgtcttgtgaccgcgggcggccgctgcttctttcccgagcttggaacttcgttatccgcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtcttctgcatgtgg

SEQ ID NO: 20 - DNA, 3278 bp, Mus musculus
  source 1..3278: mol_type = genomic DNA
gagcggggcgaacgcaaagagcgagttctccgtaggccctgcctggaagcggaagagatgggcggagataaagggcgagctgcaggccaggcggccccggagcccagggcggggcgcgggcgggccttcgctggttactgctcacaagtggcctgatctggctccaccctcggtccgggacaagtttatcttgtgactgctcgggctgcttttccttcctagattgcagagctttcatatccacgatgcgttttctggccgccacgatcctgctgctggcgctggtcgctgccagccaggcggagcccctgcacttcaaggactgcggctctaaggtgggagttataaaggaggtgaatgtgagcccatgtcccaccgatccctgtcagctgcacaaaggccagtcctacagtgtcaacatcacctttaccagcggcactcagtcccagaacagcacggccttggtccacggcatcctggaagggatccgggtccccttccctattcctgagcctgacggttgtaagagtggaatcaactgccccatccagaaagacaaggtctacagctacctgaataagcttccggtgaagaatgaatacccctctataaaactggtggtggaatggaaacttgaagatgacaaaaagaataatctcttctgctgggagatcccagttcagatcacaagctaggctccttggcgcctgtgtctgtgtgggctgagaggccatggacggagtgggggggaagaaacagaaatcagacccgaaatggaatcagtgccatatgatgaacagaatttcaagaatgctgttttatgccttttaacctccaaagcagtaactgcaagcctactactcttgagagcgcgctcagagccattgtcccccgagatagcctctggggaggcttcgggagggaaaggggaaactggaaagattagattggtgtccatggctgtttgctgttggattacaggcaggttccaccagacaggatgggacggggttgcttagggatgtcagataacttgacccagggctatggatccactgtgaaggatggcttcccagagtcttctggctgcctgggggtgttacctcccctgtttctaagtgcctcctgagtccccagcccctggcttatcagtcagatgagtctccttggtagcctctgccccatcgcttcagcagtagcgactagctctcctcggtatccagactggctgaggggcagtctgccgcagaaatttgtctctgagtggctgtgtctttgtggttagctctcgttctttggtagttttcattaaagccaatacttggttgcaggtgcttggttttgttttttaacggtatttgtctgtatcctctctctgcatgtaagattgagcctccccataaattctaacttgccactcctgcgtgtgtgttcagtctgtggttgcctacttcttccagatggcagcctcctaggtccatagcaggcagccacccagttccgaagctctagagaagatgtgactcacaggactcacgggcaccgcctctttcctcgtttttatttataccagctagactttatccatagaaagtggccttgttctgcattgctgtcagtgtgcctgaagcagcaaccaccacctagcaccacttgaggaggcgtggtgacggggacactacgcgccctcatggggaagcctagaaaggagagggaggaagaggctgtggctctatggaggcaggagggatgtgagttccaggccagtccgagtggcagcatagtgagatccatttaaataaagaagtaggcccaagccgacaaggggcagaagggtcgtgagttccacaccagcctggacttaaccactctgccgtcccacccttccctgactcaatttcagttctagaaggcaacagaggcatgaagactgctgcaagttggagaccaaccccgtctacaaaatgagctcaaggccagcctgagctaaaggagacctagtctcagatggggagagagaacatcatagcagataacagttccctttactatatctcaagatgactgacaggtatggtcatggatcatagtttttggtagggaatgcacacgctgtttttgagatgaggtctgacgtatctcaggctggctttgaactagccatgtatcggagaaccttgactttctgagcctcctgcttctacctcaagtgctgagattgccagtgtaggcccccattcataaaatccgaatagcataggactggaggcttgcgggtttttttttacctgtgagtttcccaggcaaaccaccagggggcagcaatgggtcaataatgaagttcttaccggtgacctgttcacccactgtacagaggtcttttgtgtatcctttcaccctgagcccaccctatcttcccatatgagtcagaatcatgtgaactccaaaagtagttacctgctgaggtcgttgcttccagctgcccgtggttaaattttggctcctccagttattcagctgtgaatgtgagctgtaagtgattccatctctccgtgcttcaatttggattttcctaatccattgaccagggacaatggtagcgccatgtgaagtgaaatgaggcatgtaaaacacgagtcagggcgtgcggtatacacgctgtcggtgtgagccattgtgctaggctgctctttttgtctagaaataggaaaatccggaggctcaatctcatgtgtaatgtgtctcagaaaaacatagctatcgtctgtgtgggagcttgctgaggggccaggagaaactcacttctgtgtttgctgactcccttttcccaacagattcttcttgcctttttagctctgcaactaacagctctcaaacccgaaaaaatggggggtgggaggaggaagctctgttagtgcccttgtgtcctcatcagaacaggaggtgacccaacagcttaggactgtttgcttacgggccactgggaggctcagtggagcctgcagttgtgagataggtagtctcccatgggaggattagtttgaaaggtcatccctacaagaaagttcacagtcattaacacagaagccttgaatcctaagcatacaaagggccccgtgttttctactgcctgtctctgtgcaaaccttgagtcttagacccactcttcccttacaggaatctgaggcaggtaaatctcaagatccccaatgctatggtctgatcttcatactaataaaaatcaaggggtggaatcatttcaaaaaaaaaaaaaaaaa

SEQ ID NO: 21 - protein, 1278 aa, Homo sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

SEQ ID NO: 22 - protein, 1277 aa, Mus musculus
MGAHHPALGLLLLLLCPAQVFSQSCVWYGECGIATGDKRYNCKYSGPPKPLPKDGYDLVQELCPGLFFDNVSLCCDIQQLQTLKSNLQLPLQFLSRCPSCFYNLMTLFCELTCSPHQSQFLNVTATEDYFDPKTQENKTNVKELEYFVGQSFANAMYNACRDVEAPSSNEKALGLLCGRDARACNATNWIEYMFNKDNGQAPFTIIPVFSDLSILGMEPMRNATKGCNESVDEVTGPCSCQDCSIVCGPKPQPPPPPMPWRIWGLDAMYVIMWVTYVAFLFVFFGALLAVWCHRRRYFVSEYTPIDSNIAFSVNSSDKGEASCCDPLGAAFDDCLRRMFTKWGAFCVRNPTCIIFFSLAFITVCSSGLVFVQVTTNPVELWSAPHSQARLEKEYFDKHFGPFFRTEQLIIQAPNTSVHIYEPYPAGADVPFGPPLNKEILHQVLDLQIAIESITASYNNETVTLQDICVAPLSPYNKNCTIMSVLNYFQNSHAVLDSQVGDDFYIYADYHTHFLYCVRAPASLNDTSLLHGPCLGTFGGPVFPWLVLGGYDDQNYNNATALVITFPVNNYYNDTERLQRAWAWEKEFISFVKNYKNPNLTISFTAERSIEDELNRESNSDVFTVIISYVVMFLYISLALGHIQSCSRLLVDSKISLGIAGILIVLSSVACSLGIFSYMGMPLTLIVIEVIPFLVLAVGVDNIFILVQTYQRDERLQEETLDQQLGRILGEVAPTMFLSSFSETSAFFFGALSSMPAVHTFSLFAGMAVLIDFLLQITCFVSLLGLDIKRQEKNHLDILCCVRGADDGQGSHASESYLFRFFKNYFAPLLLKDWLRPIVVAVFVGVLSFSVAVVNKVDIGLDQSLSMPNDSYVIDYFKSLAQYLHSGPPVYFVLEEGYNYSSRKGQNMVCGGMGCDNDSLVQQIFNAAELDTYTRVGFAPSSWIDDYFDWVSPQSSCCRLYNVTHQFCNASVMDPTCVRCRPLTPEGKQRPQGKEFMKFLPMFLSDNPNPKCGKGGHAAYGSAVNIVGDDTYIGATYFMTYHTILKTSADYTDAMKKARLIASNITETMRSKGSDYRVFPYSVFYVFYEQYLTIIDDTIFNLSVSLGSIFLVTLVVLGCELWSAVIMCITIAMILVNMFGVMWLWGISLNAVSLVNLVMSCGISVEFCSHITRAFTMSTKGSRVSRAEEALAHMGSSVFSGITLTKFGGIVVLAFAKSQIFEIFYFRMYLAMVLLGATHGLIFLPVLLSYIGPSVNKAKRHTTYERYRGTERERLLNF

SEQ ID NO: 23 - protein, 151 aa, Homo sapiens
MRFLAATFLLLALSTAAQAEPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 24 - protein, 149 aa, Mus musculus
MRFLAATILLLALVAASQAEPLHFKDCGSKVGVIKEVNVSPCPTDPCQLHKGQSYSVNITFTSGTQSQNSTALVHGILEGIRVPFPIPEPDGCKSGINCPIQKDKVYSYLNKLPVKNEYPSIKLVVEWKLEDDKKNNLFCWEIPVQITS

SEQ ID NO: 25 - DNA, 183 bp, synthetic construct
  misc_feature 1..183: note = Synthetic
  source 1..183: mol_type = other DNA
ttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctc

SEQ ID NO: 26 - DNA, 4276 bp, synthetic construct
  misc_feature 1..4276: note = Synthetic
  source 1..4276: mol_type = other DNA
aagagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattcgatatcgccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccagattttctacttccggatgtacctggcgatggtgcttctgggagcaacccacggcctgatcttcctgcccgtgctcttgtcctacatcggccccagcgtgaacaaggccaagtcctgcgccactgaggaacgctacaagggcaccgaaagagaaaggctgctgaatttctgatagatgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 27 - DNA, 4305 bp, synthetic construct
  misc_feature 1..4305: note = Synthetic
  source 1..4305: mol_type = other DNA
aagagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattcgatatcgccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgcttctgtcctacatcggcccgtccgtgaacaaagcgaagtcctgtgctactgaggagcggtacaagggaactgagcgcgagcgcctgctcaactttgggggcggccgcaagaagcgccggcagcgcagaaggtgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 28 - DNA, 4354 bp, synthetic construct
  misc_feature 1..4354: note = Synthetic
  source 1..4354: mol_type = other DNA
aagagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattcgatatcgccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggaaccgagagggagcgccttctcaacttcggaggagattataaagacgatgatgacaagggggactacaaggacgacgatgacaagggcgattacaaagacgacgacgacaagttgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 29 - DNA, 4339 bp, synthetic construct
  misc_feature 1..4339: note = Synthetic
  source 1..4339: mol_type = other DNA
aagagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattcgatatcgccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaaggggactgaacgcgaacggctgctgaactttggcggcgctgggtacctgctcggaaagatcaacctcaaagcgctggcggccctggccaagaagatcctgttgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 30 - DNA, 4312 bp, synthetic construct
  misc_feature 1..4312: note = Synthetic
  source 1..4312: mol_type = other DNA
aagagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattcgatatcgccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggtactgaaagagagcggctgctcaacttcggtggccccctgatctacctgcgcctcctgcggggtcagttcttgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 31 - DNA, 4387 bp, synthetic construct
  misc_feature 1..4387: note = Synthetic
  source 1..4387: mol_type = other DNA
aagagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattcgatatcgccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggaaccgagagggagcgccttctcaacttcggaggagattataaagacgatgatgacaagggggactacaaggacgacgatgacaagggcgattacaaagacgacgacgacaagggcggccggaagaagcgccgccagcggcggagattgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 32 - DNA, 4423 bp, synthetic construct
  misc_feature 1..4423: note = Synthetic
  source 1..4423: mol_type = other DNA
aagagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattcgatatcgccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggaaccgagagggagcgccttctcaacttcggaggagattataaagacgatgatgacaagggggactacaaggacgacgatgacaagggcgattacaaagacgacgacgacaagggcggcgccggctacctcctggggaagattaacctgaaggccctggcagccctcgccaagaagatcctgttgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 33 - DNA, 4396 bp, synthetic construct
  misc_feature 1..4396: note = Synthetic
  source 1..4396: mol_type = other DNA
aagagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacgcgtaagggcgaattcgatatcgccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggaaccgagagggagcgccttctcaacttcggaggagattataaagacgatgatgacaagggggactacaaggacgacgatgacaagggcgattacaaagacgacgacgacaaggggggcccgctcatctacctccggctgctgcggggccagtttttgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 34 - DNA, 4276 bp, synthetic construct; identical to SEQ ID NO: 26
  misc_feature 1..4276: note = Synthetic
  source 1..4276: mol_type = other DNA

SEQ ID NO: 35 - DNA, 4305 bp, synthetic construct; identical to SEQ ID NO: 27
  misc_feature 1..4305: note = Synthetic
  source 1..4305: mol_type = other DNA

SEQ ID NO: 36 - DNA, 4354 bp, synthetic construct; identical to SEQ ID NO: 28
  misc_feature 1..4354: note = Synthetic
  source 1..4354: mol_type = other DNA

SEQ ID NO: 37 - DNA, 4387 bp, synthetic construct; identical to SEQ ID NO: 31
  misc_feature 1..4387: note = Synthetic
  source 1..4387: mol_type = other DNA

SEQ ID NO: 38 - DNA, 4276 bp, synthetic construct
  misc_feature 1..4276: note = Synthetic
  source 1..4276: mol_type = other DNA
cggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccagattttctacttccggatgtacctggcgatggtgcttctgggagcaacccacggcctgatcttcctgcccgtgctcttgtcctacatcggccccagcgtgaacaaggccaagtcctgcgccactgaggaacgctacaagggcaccgaaagagaaaggctgctgaatttctgatagatgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 39 - DNA, 4305 bp, synthetic construct
  misc_feature 1..4305: note = Synthetic
  source 1..4305: mol_type = other DNA
cggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgcttctgtcctacatcggcccgtccgtgaacaaagcgaagtcctgtgctactgaggagcggtacaagggaactgagcgcgagcgcctgctcaactttgggggcggccgcaagaagcgccggcagcgcagaaggtgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 40 - DNA, 4354 bp, synthetic construct
  misc_feature 1..4354: note = Synthetic
  source 1..4354: mol_type = other DNA
cggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcccgccaccatgaccgcgaggggactggccctcgggctgctcctgctgctcctttgcccggcccaagtgttctcgcaatcgtgcgtgtggtacggggaatgcggaattgcctacggcgacaagcggtacaactgcgaatactccgggcctccaaagccgctgcccaaggacggatacgacctggtccaagagctctgccctggattcttcttcggcaacgtgtcgctgtgttgtgacgtgcggcagctgcagaccctgaaggataacctccagctgccgctgcaattcctctcccggtgtccgtcatgcttctacaacttgctcaacctgttctgcgaacttacctgttcgccgagacagtcccagtttcttaacgtcaccgcaaccgaggactacgtggaccctgtgactaaccagaccaagactaatgtcaaagaattgcagtactacgtgggccagtccttcgccaatgcgatgtacaacgcctgtcgggacgtcgaagcgcccagctccaacgacaaggcactcggactgctttgcggcaaagatgccgacgcctgcaacgccaccaactggatcgagtacatgttcaataaggacaacggacaggcccctttcaccatcacacctgtgttcagcgacttcccagtccacgggatggaacctatgaacaacgcgactaagggatgcgacgagtccgtggacgaagtgaccgccccgtgttcctgtcaagattgctcaatcgtgtgcggtccgaagccccagcctcctcctccgccggctccatggaccatcctgggtctggacgctatgtacgtgattatgtggatcacttacatggccttcctcctggtgttcttcggcgctttcttcgccgtgtggtgttaccggaagcgctacttcgtgtccgagtatactcctatcgactctaacatagcgttctccgtgaacgcctccgataagggagaggcatcgtgctgtgatcccgtgtccgccgctttcgaaggatgcttgaggcggctgttcacccgctggggcagcttctgcgtgagaaatcccggctgtgtgattttcttctcgctggtgttcatcaccgcttgctcgtcggggctggtgttcgtcagagtcacaacgaaccccgtggacctgtggagcgcgcctagcagccaggcccgcctggaaaaggaatacttcgaccagcatttcggaccctttttccggaccgaacagctgattatccgcgccccgctgactgacaagcatatctaccagccttacccgagcggagcggatgtgccctttggtccgccactcgacatccagatcctgcaccaagtcctggacttgcaaatcgctattgagaacattactgcctcctacgacaacgagacagtgaccctgcaggacatttgtcttgccccgctgtccccctacaacaccaactgcacgatcctgagcgtgctgaactatttccaaaactcgcactccgtgctggatcacaagaagggcgacgatttcttcgtctacgccgattaccacacccacttcctgtactgcgtgcgcgctccggcttcactgaacgacacttccctcctccacgacccgtgcctgggaacgttcggcggacccgtgtttccctggctggtcctgggcggctacgacgaccagaactacaacaacgccaccgccctcgtgatcacctttcctgtgaacaactactacaacgacaccgaaaagttgcagagagcacaagcgtgggagaaggagttcatcaacttcgtgaagaactacaaaaaccctaacctgaccatctcctttacggccgagcgctcaatcgaggacgaattgaacagggaatcggactccgacgtgttcactgtcgtcattagctacgccatcatgttcttgtacattagcctggcgctggggcacatgaagtcctgccgccggctgctggtcgattcgaaggtgtccctgggaatcgccggcatcctgatcgtcctgtcgtccgtggcctgttccctgggagtgttcagctacattggactgccactgaccctcattgtgattgaagtgatcccttttcttgtcttggccgtgggagtggataatatttttatcctggtgcaagcctaccagcgggacgagaggctgcagggggagactctggaccagcagctgggccgcgtgctgggcgaagtcgccccgtccatgtttctgtcctcattctccgaaaccgtggccttcttcctgggcgcgctcagcgtgatgcctgccgtgcacaccttctccctgtttgccggtctggctgtgtttatcgatttccttctccaaattacttgcttcgtgtcactgcttggactcgacatcaagcgccaggaaaagaaccggctggacattttctgctgcgtgagaggagcggaggacggaacctcagtgcaggcttccgagtcatgcctgtttcgattcttcaagaactcgtattcgccgctgctcctgaaggattggatgcggcccattgtgatcgccatctttgtcggcgtcctgagcttcagcattgcggtgttgaacaaagtggacattggcctggaccagagcctctccatgccggatgattcctacatggtggactacttcaagagcatctctcagtacttgcacgctggccctcccgtgtatttcgtgctggaggaagggcacgactacactagctctaagggacagaacatggtctgcggtggcatgggatgcaacaatgactcgctggtgcagcagattttcaacgccgcgcaactcgataactacaccaggattggattcgctccctcctcctggatcgacgattattttgactgggtcaagccgcagtctagctgctgccgggtggacaacatcacggatcagttctgcaatgcttccgtggtcgacccggcctgcgtgcggtgccggcctcttactccggagggaaagcagaggccccagggtggcgacttcatgcggtttctgcccatgttcctgagcgataaccccaaccccaaatgcgggaagggaggtcacgcggcgtactcgtcagcggtcaacatcctgctgggccatggaactagagtgggagcgacctacttcatgacttaccatactgtgctgcagacctcagccgacttcatcgacgcactcaagaaagcccgcctgatcgcatcaaacgtgaccgagactatggggatcaacggatccgcgtaccgcgtgttcccatattcggtgttctacgtgttctacgagcaatacctgaccattattgacgacaccatctttaacctgggggtgtcactgggagccatcttccttgtgaccatggtcctcctgggctgcgaactgtggtccgccgtgatcatgtgcgctaccatcgcaatggtcctcgtgaacatgtttggggtcatgtggctgtggggcatctccctgaacgcggtgtccctcgtgaacctggtcatgtcatgcggcattagcgtggagttctgctcccacattactcgcgccttcaccgtgtcgatgaagggttcccgcgtggagcgggcggaggaagccctggcccacatgggatcctcggtgttctcgggcatcacccttactaagttcggcggtatcgtggtgctggccttcgccaagtcacagatcttccaaattttctacttcagaatgtacttggcgatggtcctgcttggagccacacacggtctgatcttcctgcctgtgctgctgagctacatcggtcccagcgtgaacaaggctaagtcctgcgcgacggaggaaaggtacaagggaaccgagagggagcgccttctcaacttcggaggagattataaagacgatgatgacaagggggactacaaggacgacgatgacaagggcgattacaaagacgacgacgacaagttgatagaagcttctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 41 - protein, 1278 aa, Homo sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHIKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

SEQ ID NO: 42 - protein, 1332 aa, Homo sapiens
MAEAGLRGWLLWALLLRLAQSEPYTTIHQPGYCAFYDECGKNPELSGSLMTLSNVSCLSNTPARKITGDHLILLQKICPRLYTGPNTQACCSAKQLVSLEASLSITKALLTRCPACSDNFVNLHCHNTCSPNQSLFINVTRVAQLGAGQLPAVVAYEAFYQHSFAEQSYDSCSRVRVPAAATLAVGTMCGVYGSALCNAQRWLNFQGDTGNGLAPLDITFHLLEPGQAVGSGIQPLNEGVARCNESQGDDVATCSCQDCAASCPAIARPQALDSTFYLGQMPGSLVLIIILCSVFAVVTILLVGFRVAPARDKSKMVDPKKGTSLSDKLSFSTHTLLGQFFQGWGTWVASWPLTILVLSVIPVVALAAGLVFTELTTDPVELWSAPNSQARSEKAFHDQHFGPFFRTNQVILTAPNRSSYRYDSLLLGPKNFSGILDLDLLLELLELQERLRHLQVWSPEAQRNISLQDICYAPLNPDNTSLYDCCINSLLQYFQNNRTLLLLTANQTLMGQTSQVDWKDHFLYCANAPLTFKDGTALALSCMADYGAPVFPFLAIGGYKGKDYSEAEALIMTFSLNNYPAGDPRLAQAKLWEEAFLEEMRAFQRRMAGMFQVTFMAERSLEDEINRTTAEDLPIFATSYIVIFLYISLALGSYSSWSRVMVDSKATLGLGGVAVVLGAVMAAMGFFSYLGIRSSLVILQVVPFLVLSVGADNIFIFVLEYQRLPRRPGEPREVHIGRALGRVAPSMLLCSLSEAICFFLGALTPMPAVRTFALTSGLAVILDFLLQMSAFVALLSLDSKRQEASRLDVCCCVKPQELPPPGQGEGLLLGFFQKAYAPFLLHWITRGVVLLLFLALFGVSLYSMCHISVGLDQELALPKDSYLLDYFLFLNRYFEVGAPVYFVTTLGYNFSSEAGMNAICSSAGCNNFSFTQKIQYATEFPEQSYLAIPASSWVDDFIDWLTPSSCCRLYISGPNKDKFCPSTVNSLNCLKNCMSITMGSVRPSVEQFHKYLPWFLNDRPNIKCPKGGLAAYSTSVNLTSDGQVLASRFMAYHKPLKNSQDYTEALRAARELAANITADLRKVPGTDPAFEVFPYTITNVFYEQYLTILPEGLFMLSLCLVPTFAVSCLLLGLDLRSGLLNLLSIVMILVDTVGFMALWGISYNAVSLINLVSAVGMSVEFVSHITRSFAISTKPTWLERAKEATISMGSAVFAGVAMTNLPGILVLGLAKAQLIQIFFFRLNLLITLLGLLHGLVFLPVILSYVGPDVNPALALEQKRAEEAVAAVMVASCPNHPSRVSTADNIYVNHSFEGSIKGAGAISNFLPNNGRQF

SEQ ID NO: 43 - protein, 1286 aa, Homo sapiens
MAEAGLRGWLLWALLLRLAQSEPYTTIHQPGYCAFYDECGKNPELSGSLMTLSNVSCLSNTPARKITGDHLILLQKICPRLYTGPNTQACCSAKQLVSLEASLSITKALLTRCPACSDNFVNLHCHNTCSPNQSLFINVTRVAQLGAGQLPAVVAYEAFYQHSFAEQSYDSCSRVRVPAAATLAVGTMCGVYGSALCNAQRWLNFQGDTGNGLAPLDITFHLLEPGQAVGSGIQPLNEGVARCNESQGDDVATCSCQDCAASCPAIARPQALDSTFYLGQMPGSLVLIIILCSVFAVVTILLVGFRVAPARDKSKMVDPKKGTSLSDKLSFSTHTLLGQFFQGWGTWVASWPLTILVLSVIPVVALAAGLVFTELTTDPVELWSAPNSQARSEKAFHDQHFGPFFRTNQVILTAPNRSSYRYDSLLLGPKNFSGILDLDLLLELLELQERLRHLQVWSPEAQRNISLQDICYAPLNPDNTSLYDCCINSLLQYFQNNRTLLLLTANQTLMGQTSQVDWKDHFLYCANAPLTFKDGTALALSCMADYGAPVFPFLAIGGYKGKDYSEAEALIMTFSLNNYPAGDPRLAQAKLWEEAFLEEMRAFQRRMAGMFQVTFMAERSLEDEINRTTAEDLPIFATSYIVIFLYISLALGSYSSWSRVMVDSKATLGLGGVAVVLGAVMAAMGFFSYLGIRSSLVILQVVPFLVLSVGADNIFIFVLEYQRLPRRPGEPREVHIGRALGRVAPSMLLCSLSEAICFFLGALTPMPAVRTFALTSGLAVILDFLLQMSAFVALLSLDSKRQEASRLDVCCCVKPQELPPPGQGEGLLLGFFQKAYAPFLLHWITRGVVVGAPVYFVTTLGYNFSSEAGMNAICSSAGCNNFSFTQKIQYATEFPEQSYLAIPASSWVDDFIDWLTPSSCCRLYISGPNKDKFCPSTVNSLNCLKNCMSITMGSVRPSVEQFHKYLPWFLNDRPNIKCPKGGLAAYSTSVNLTSDGQVLASRFMAYHKPLKNSQDYTEALRAARELAANITADLRKVPGTDPAFEVFPYTITNVFYEQYLTILPEGLFMLSLCLVPTFAVSCLLLGLDLRSGLLNLLSIVMILVDTVGFMALWGISYNAVSLINLVSAVGMSVEFVSHITRSFAISTKPTWLERAKEATISMGSAVFAGVAMTNLPGILVLGLAKAQLIQIFFFRLNLLITLLGLLHGLVFLPVILSYVGPDVNPALALEQKRAEEAVAAVMVASCPNHPSRVSTADNIYVNHSFEGSIKGAGAISNFLPNNGRQF

SEQ ID NO: 44 - protein, 1276 aa, Felis catus
MTARGPALGLLLLLLCPAQVLAQSCIWYGECGIASGDKRYNCKYSGPPKPLPKDGYDLVQELCPGFFFDNVSLCCDVQQLQTLKDNLQLPLQFLSRCPSCFYNLVNLFCELTCSPRQSQFLNVTATEDYVDPVTNQTKTNVKELQYYIGESFANAMYNACRDVEAPSSNDKALGLLCGKDAEACNATNWIEYMFSKDNGQAPFTITPIFSDLPTHGMEPMNNATKGCDESVDEVTGPCSCQDCSIVCGPKPQPPPPPVPWRILGLDAMYVIMWITYMAFLLVFFGAFFALWCYRKRYFVSEYTPIDSNIAFSVNANDRGEASCCDALGAAFEGCLRRLFSQWGSFCVRNPGPIIFFSLAFIAACSSGLVFVRVTTNPVDLWSAPSSQARLEKEYFDTHFGPFFRTEQLIIQAPHTSAHTYQPYPSGSDVPFGPPLDLAILHQVLDLQTAIENITASYNNETVTLQDICVAPLSPYNKNCTILSVLNYFQNSHSMLDHEIGDDFFVYADYHTHLLYCVRAPASLNDTSLLHDPCLGTFGGPVFPWLVLGGYDDQNYNNATALVITFPVNNYYNDTERLQKAHVWEKEFINFVKNYKNPNLTISFTTERSIEDELNRESNGDIFTVIISYAIMFLYISIALGHIKSCSRLLVDSKISLGIAGILIVLSSKACSLGIFSYVGIPLTLIVIEVIPFLVLAVGVDNIFILVQTYQRDERLHGETLDQQLGRVLGEVAPSMFLSSFSEAVAFFLGALSKMPAVHTFSLFAGMAVLIDFLLQITCFVSLLGLDIKRQEKNRLDVLCCVRGSEDGTSVQASESCLFRLFKHSYSPLLLKDWMRPIVIAIFVGVLSFSVAVLNKVEIGLDQSLSMPDDSYVMDYFKSLKYLHAGPPVYFVLEEGHDYTSLKGQNMVCGGMGCNNDSLVQQIFNAAQLDSYTRIGFAPSSWIDDYFDWVKPQSSCCRVYNSTDRFCNASVVDPACIRCRPLTQEGKQRPQGGDFMRFLPMFLSDNPNPKCGKGGHAAYSSAVNILGNDTGVGATYFMTYHTVLQTSADFTDAMRKANLIASNITKTMGLEGSNYRVFPYSVFYVFYEQYLTIIDDTIFNLSVSLGAIFLVTVILLGCDLWSAVIMCITIAMILVNMFGVMWLWGISLNAVSLVNLVMSCGISVEFCSHITRAFTVSMKGSRAQRAEEALAHMGSSVFSGITLTKFGGIVVLAFAKSQIFQIFYFRMYLAMVLLGATHGLIFLPVLLSYIGPSINKAKSLATQEQYKGTEREQLLNF

SEQ ID NO: 45 - protein, 1277 aa, Mus musculus
MGAHHPALGLLLLLLCPAQVFSQSCVWYGECGIATGDKRYNCKYSGPPKPLPKDGYDLVQELCPGLFFDNVSLCCDIQQLQTLKSNLQLPLQFLSRCPSCFYNLMTLFCELTCSPHQSQFLNVTATEDYFDPKTQENKTNVKELEYFVGQSFANAMYNACRDVEAPSSNEKALGLLCGRDARACNATNWIEYMFNKDNGQAPFTIIPVFSDLSILGMEPMRNATKGCNESVDEVTGPCSCQDCSIVCGPKPQPPPPPMPWRIWGLDAMYVIMWVTYVAFLFVFFGALLAVWCHRRRYFVSEYTPIDSNIAFSVNSSDKGEASCCDPLGAAFDDCLRRMFTKWGAFCVRNPTCIIFFSLAFITVCSSGLVFVQVTTNPVELWSAPHSQARLEKEYFDKHFGPFFRTEQLIIQAPNTSVHIYEPYPAGADVPFGPPLNKEILHQVLDLQIAIESITASYNNETVTLQDICVAPLSPYNKNCTIMSVLNYFQNSHAVLDSQVGDDFYIYADYHTHFLYCVRAPASLNDTSLLHGPCLGTFGGPVFPWLVLGGYDDQNYNNATALVITFPVNNYYNDTERLQRAWAWEKEFISFVKNYKNPNLTISFTAERSIEDELNRESNSDVFTVIISYVVMFLYISLALGHIQSCSRLLVDSKISLGIAGILIVLSSVACSLGIFSYMGMPLTLIVIEVIPFLVLAVGVDNIFILVQTYQRDERLQEETLDQQLGRILGEVAPTMFLSSFSETSAFFFGALSSMPAVHTFSLFAGMAVLIDFLLQITCFVSLLGLDIKRQEKNHLDILCCVRGADDGQGSHASESYLFRFFKNYFAPLLLKDWLRPIVVAVFVGVLSFSVAVVNKVDIGLDQSLSMPNDSYVIDYFKSLAQYLHSGPPVYFVLEEGYNYSSRKGQNMVCGGMGCDNDSLVQQIFNAAELDTYTRVGFAPSSWIDDYFDWVSPQSSCCRLYNVTHQFCNASVMDPTCVRCRPLTPEGKQRPQGKEFMKFLPMFLSDNPNPKCGKGGHAAYGSAVNIVGDDTYIGATYFMTYHTILKTSADYTDAMKKARLIASNITETMRSKGSDYRVFPYSVFYVFYEQYLTIIDDTIFNLSVSLGSIFLVTLVVLGCELWSAVIMCITIAMILVNMFGVMWLWGISLNAVSLVNLVMSCGISVEFCSHITRAFTMSTKGSRVSRAEEALAHMGSSVFSGITLTKFGGIVVLAFAKSQIFEIFYFRMYLAMVLLGATHGLIFLPVLLSYIGPSVNKAKRHTTYERYRGTERKRLLNF

SEQ ID NO: 46 - protein, 1277 aa, Bos taurus
MSVRGPAFGLLLLLFCPAQVFSQSCIWYGECGIASGDKRYNCRYSGPPEPLPQDGYDLVQELCPGFFFGNVSLCCDVQQLHTLKDNLQLPLQFLSRCPSCFYNLVNLFCELTCSPRQSQFLNVTATEDYVDPATNQTKTNVKELQYYVGESFANAMYNSCRDVEAPSSNEKALGLLCGREASACNATNWIEYMFNKDNGQAPFTITPVFSDLPTHGMEPMNNATKGCDESVDEVTGPCSCQDCSAVCGPKPQPPPPPVPWRILGLDAMYVIMWSTYMAFLLVFFGAFFAVWCYRKRYFVSEFTPIDGNIPFSINASDKGGPTCCDPLGAAFEAHLRRLFEWWGSFCVRHPGCVVFFSVAFIAACSSGLVFIQVTTDPVDLWSAPGSQARLEKEYFDTHFGPFFRTEQLIIRAPHTPPHIYEPYPSGADVPFGPPLAVDILHQVLDLQTAIESITASYNNETVTLRDICVAPLSPYNQNCTILSVLNYFQNSHSVLDHQVGDDFFVYADYHTHFLYCVRAPASLNDTSLLHDPCLGTFGGPVFPWLVLGGYDDQNYNNATALVITFPVNNYYNDTEKLQRAQAWEREFINFVQNYENPNLTISFKAERSIEDELNRESNSDVFTVLISYGVMFLYISIALGHIKSCRRLLVDSKISLGIAGVLIVLSSVACSLGIFSYIGVPLTLIVIEVIPFLVLAVGVDNIFILVQTYQRDERLQGETLDQQVGRVLGEVAPSMFLSSFAETVAFFLGGLSVMPAVHTFSLFAGMAVLIDFLLQITCFVSLLGLDIKRQEKNQLDVLCCVGGAADDAGIQASESCLFRFFRNSYAPLLLKDWMRPLVVAVFVGVLSFSIAVLNKVEIGLDQSLSMPDDSYVTDYFQSLNQYLHAGPPVYFVLEEGHDYTSTKGQNMVCGGLGCNNDSLVQQVFTAAQLDSYTRIGFAPSSWIDDYFDWVKPQSSCCRIYNSTEQFCNASVVNPTCVRCRPLTPEGKQRPQGADFMRFLPMFLSDNPNPKCGKGGHAAYSAAVNILDNGTRVGATYFMTYHTVLQTSADFIDAMEKARLIASNITRTMNQQGGDHRVFPYSVFYVFYEQYLTMIDDTIFNLSVSLGAIFLVAVVLLGCELWSAVIMCATIAMILVNMFGVMWLWGISLNAVSLVNLVMSCGISVEFCSHITRAFTVSTKGSRVERAEEALSHMGSSVFSGITLTKFGGIIVLAFAKSQIFQIFYFRMYLAMVLLGATHGLIFLPVLLSYIGPSINKAKSLTTQQRYRGTEREQLLNF

SEQ ID NO: 47 - protein, 91 aa, Danio rerio
GEEALAHMGSSVFSGITLTKFGGILILALSKSQIFQIFYFRMYLAIVLLGAAHGLIFLPVLLSYAGPSVNKAKVLAAHNRFVGTERERLIY

SEQ ID NO: 48 - DNA, 130 bp, synthetic construct
  misc_feature 1..130: note = Synthetic
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 49 - DNA, 145 bp, synthetic construct
  misc_feature 1..145: note = Synthetic
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 50 - DNA, 59 bp, synthetic construct
  misc_feature 1..59: note = Synthetic
  source 1..59: mol_type = other DNA
tgtagttaatgattaacccgccatgctacttatctaccagggtaatggggatcctctag

SEQ ID NO: 51 - DNA, 230 bp, synthetic construct
  misc_feature 1..230: note = Synthetic
  source 1..230: mol_type = other DNA
agagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaaca

SEQ ID NO: 52 - DNA, 231 bp, synthetic construct
  misc_feature 1..231: note = Synthetic
  source 1..231: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag